SEQUENCE LISTING (327 sequences)

SEQ ID NO: 1 - DNA, 347 bp, Homo sapiens
aggtgcagctgcaggagtcgggcccaggactggtgaagccttcggagaccctgtccctcacctgcactgtctctggtgactccatcagtaattactactggagctggatccggcagcccccagggaagggactggagtggattgggtatttctattacagtgggagcaccaactacaacccctccctcaagagtcgagtcaccatatcagtagacacgtccaagaaccagttctccctgaaactgagctctgtgaccgctgcggacacggccgtgtattactgtgcgagagataggggctgggctgactactggggccagggaaccctggtcaccgtctcctcagcc

SEQ ID NO: 2 - protein, 116 aa, Homo sapiens
QVQLQESGPGLVKPSETLSLTCTVSGDSISNYYWSWIRQPPGKGLEWIGYFYYSGSTNYNPSLKSRVTISVDTSKNQFSLKLSSVTAADTAVYYCARDRGWADYWGQGTLVTVSSA

SEQ ID NO: 3 - protein, 25 aa, Homo sapiens
QVQLQESGPGLVKPSETLSLTCTVS

SEQ ID NO: 4 - protein, 10 aa, Homo sapiens
GDSISNYYWS

SEQ ID NO: 5 - protein, 14 aa, Homo sapiens
WIRQPPGKGLEWIG

SEQ ID NO: 6 - protein, 16 aa, Homo sapiens
YFYYSGSTNYNPSLKS

SEQ ID NO: 7 - protein, 32 aa, Homo sapiens
RVTISVDTSKNQFSLKLSSVTAADTAVYYCAR

SEQ ID NO: 8 - protein, 7 aa, Homo sapiens
DRGWADY

SEQ ID NO: 9 - protein, 12 aa, Homo sapiens
WGQGTLVTVSSA

SEQ ID NO: 10 - DNA, 324 bp, Homo sapiens
gaaattgtgttgacgcagtctccaggcaccctgtctttgtctccaggggaaagggccaccctctcctgcagaaccagtcagagtattagcagcagctatttagcctggtaccagcagaaacctggccaggttcccaggctcctcatctatggtgcttccagcagggccactggcatcccagacaggttcagtggcagtgggtctgggacagacttcactctcaccatcagcagactggagcctgaagattttgcagtgtattattgtcagcagtatggtagctcgatcaccttcggccaagggacacgactggagattaaacga

SEQ ID NO: 11 - protein, 108 aa, Homo sapiens
EIVLTQSPGTLSLSPGERATLSCRTSQSISSSYLAWYQQKPGQVPRLLIYGASSRATGIPDRFSGSGSGTDFTLTISRLEPEDFAVYYCQQYGSSITFGQGTRLEIKR

SEQ ID NO: 12 - protein, 23 aa, Homo sapiens
EIVLTQSPGTLSLSPGERATLSC

SEQ ID NO: 13 - protein, 12 aa, Homo sapiens
RTSQSISSSYLA

SEQ ID NO: 14 - protein, 15 aa, Homo sapiens
WYQQKPGQVPRLLIY

SEQ ID NO: 15 - protein, 7 aa, Homo sapiens
GASSRAT

SEQ ID NO: 16 - protein, 32 aa, Homo sapiens
GIPDRFSGSGSGTDFTLTISRLEPEDFAVYYC

SEQ ID NO: 17 - protein, 8 aa, Homo sapiens
QQYGSSIT

SEQ ID NO: 18 - protein, 11 aa, Homo sapiens
FGQGTRLEIKR

SEQ ID NO: 19 - DNA, 360 bp, Homo sapiens
caggtgcagctgcaggagtcgggcccaggactggtgaagccttcacagaccctgtccctcacctgcactgtctctggtggctccatcagcagttttaattactactggagctggatccgccaccacccagggaagggcctggagtggattgggtacatctattacagtgggagcacctactccaacccgtccctcaagagtcgagttaccatatcagtagacacgtctaagaaccagttctccctgacgctgagctctgtgactgccgcggacacggccgtgtattactgtgcgagagggtataactggaactactttgactactggggccagggaaccctggtcaccgtctcctcagcc

SEQ ID NO: 20 - protein, 120 aa, Homo sapiens
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSA

SEQ ID NO: 21 - protein, 30 aa, Homo sapiens
QVQLQESGPGLVKPSQTLSLTCTVSGGSIS

SEQ ID NO: 22 - protein, 7 aa, Homo sapiens
SFNYYWS

SEQ ID NO: 23 - protein, 14 aa, Homo sapiens
WIRHHPGKGLEWIG

SEQ ID NO: 24 - protein, 16 aa, Homo sapiens
YIYYSGSTYSNPSLKS

SEQ ID NO: 25 - protein, 32 aa, Homo sapiens
RVTISVDTSKNQFSLTLSSVTAADTAVYYCAR

SEQ ID NO: 26 - protein, 9 aa, Homo sapiens
GYNWNYFDY

SEQ ID NO: 27 - protein, 12 aa, Homo sapiens
WGQGTLVTVSSA

SEQ ID NO: 28 - DNA, 327 bp, Homo sapiens
gaaatagtgatgacgcagtctccagccaccctgtctgtgtctccaggggaaagagccaccctctcctgcagggccagtcagagtgttgacaacaacttagtctggtaccagcagaaacctggccaggctcccaggctcctcatctatggtgcatccaccagggccactggtatcccagccaggttcagtggcagtgggtctgggacagagttcactctcaccatcagtagtctgcagtctgaagattttgcagtttattactgtcagcagtataataactggcctccgtggacgttcggccaagggaccaaggtggaaatcaaacga

SEQ ID NO: 29 - protein, 109 aa, Homo sapiens
EIVMTQSPATLSVSPGERATLSCRASQSVDNNLVWYQQKPGQAPRLLIYGASTRATGIPARFSGSGSGTEFTLTISSLQSEDFAVYYCQQYNNWPPWTFGQGTKVEIKR

SEQ ID NO: 30 - protein, 23 aa, Homo sapiens
EIVMTQSPATLSVSPGERATLSC

SEQ ID NO: 31 - protein, 11 aa, Homo sapiens
RASQSVDNNLV

SEQ ID NO: 32 - protein, 15 aa, Homo sapiens
WYQQKPGQAPRLLIY

SEQ ID NO: 33 - protein, 7 aa, Homo sapiens
GASTRAT

SEQ ID NO: 34 - protein, 32 aa, Homo sapiens
GIPARFSGSGSGTEFTLTISSLQSEDFAVYYC

SEQ ID NO: 35 - protein, 10 aa, Homo sapiens [sequence omitted]

SEQ ID NO: 36 - protein, 11 aa, Homo sapiens
FGQGTKVEIKR

SEQ ID NO: 37 - DNA, 369 bp, Homo sapiens
atcaccttgaaggagtctggtcctacgctggtgaaacccacacagaccctcacgctgacctgcaccttctctgggttctcactcagcgctggtggagtgggtgtgggctggatccgtcagcccccaggaaaggccctggagtggcttgcactcatttattggaatgatgataagcgctacagcccatctctgaggagcaggctcaccatcaccaaggacacctccaaaaaccaggtggtccttacaattaccaacatggaccctgtggacacagccacatattattgtgcacacagtcactatgattacgattgggggagttactttgactactggggccagggaaccctggtcaccgtctcctcagcc

SEQ ID NO: 38 - protein, 123 aa, Homo sapiens
ITLKESGPTLVKPTQTLTLTCTFSGFSLSAGGVGVGWIRQPPGKALEWLALIYWNDDKRYSPSLRSRLTITKDTSKNQVVLTITNMDPVDTATYYCAHSHYDYDWGSYFDYWGQGTLVTVSSA

SEQ ID NO: 39 - protein, 24 aa, Homo sapiens
ITLKESGPTLVKPTQTLTLTCTFS

SEQ ID NO: 40 - protein, 12 aa, Homo sapiens
GFSLSAGGVGVG

SEQ ID NO: 41 - protein, 14 aa, Homo sapiens
WIRQPPGKALEWLA

SEQ ID NO: 42 - protein, 16 aa, Homo sapiens
LIYWNDDKRYSPSLRS

SEQ ID NO: 43 - protein, 32 aa, Homo sapiens
RLTITKDTSKNQVVLTITNMDPVDTATYYCAH

SEQ ID NO: 44 - protein, 13 aa, Homo sapiens
SHYDYDWGSYFDY

SEQ ID NO: 45 - protein, 12 aa, Homo sapiens
WGQGTLVTVSSA

SEQ ID NO: 46 - DNA, 342 bp, Homo sapiens
gatattgtgatgacccagactccactctccctgcccgtcacccctggagagccggcctccatctcctgcaggtctagtcagagcctcttggatagtgatgatggaaacacctatttggactggtacctgcagaagccaggacagtctccacagctcctgatctatacgctttcctatcgggcctctggagtcccagacaggttcagtggcagtgggtcaggcactgatttcacactgaacatcagcagggtggaggctgaggatgttggagtttattactgcatgcaacgtatagagtttcctatcaccttcggccaagggacacgactggagattaaacga

SEQ ID NO: 47 - protein, 114 aa, Homo sapiens
DIVMTQTPLSLPVTPGEPASISCRSSQSLLDSDDGNTYLDWYLQKPGQSPQLLIYTLSYRASGVPDRFSGSGSGTDFTLNISRVEAEDVGVYYCMQRIEFPITFGQGTRLEIKR

SEQ ID NO: 48 - protein, 23 aa, Homo sapiens
DIVMTQTPLSLPVTPGEPASISC

SEQ ID NO: 49 - protein, 17 aa, Homo sapiens
RSSQSLLDSDDGNTYLD

SEQ ID NO: 50 - protein, 15 aa, Homo sapiens
WYLQKPGQSPQLLIY

SEQ ID NO: 51 - protein, 7 aa, Homo sapiens
TLSYRAS

SEQ ID NO: 52 - protein, 32 aa, Homo sapiens
GVPDRFSGSGSGTDFTLNISRVEAEDVGVYYC

SEQ ID NO: 53 - protein, 9 aa, Homo sapiens
MQRIEFPIT

SEQ ID NO: 54 - protein, 11 aa, Homo sapiens
FGQGTRLEIKR

SEQ ID NO: 55 - DNA, 360 bp, Homo sapiens
caggtgcagctgcaggagtcgggcccaggactggtgaagccttcacagaccctgtccctcacctgcactgtctctggtggctccatcagcagtgctaattactactggacctggatccgccagcacccagggaagggcctggagtggattgggtacatctattacagtgggagcacctactgcaacccgtccctcaagagtcgagttatcatatcagtagacacgtctaagaaccagttctccctgaagctgagctctgtgactgccgcggacacggccgtgtattactgtgcgagagggtataactggaactactttgactactggggccagggaaccctggtcaccgtctcctcagcc

SEQ ID NO: 56 - protein, 120 aa, Homo sapiens
QVQLQESGPGLVKPSQTLSLTCTVSGGSISSANYYWTWIRQHPGKGLEWIGYIYYSGSTYCNPSLKSRVIISVDTSKNQFSLKLSSVTAADTAVYYCARGYNWNYFDYWGQGTLVTVSSA

SEQ ID NO: 57 - protein, 24 aa, Homo sapiens
QVQLQESGPGLVKPSQTLSLTCTV

SEQ ID NO: 58 - protein, 12 aa, Homo sapiens
GGSISSANYYWT

SEQ ID NO: 59 - protein, 14 aa, Homo sapiens
WIRQHPGKGLEWIG

SEQ ID NO: 60 - protein, 16 aa, Homo sapiens
YIYYSGSTYCNPSLKS

SEQ ID NO: 61 - protein, 32 aa, Homo sapiens
RVIISVDTSKNQFSLKLSSVTAADTAVYYCAR

SEQ ID NO: 62 - protein, 9 aa, Homo sapiens
GYNWNYFDY

SEQ ID NO: 63 - protein, 12 aa, Homo sapiens
WGQGTLVTVSSA

SEQ ID NO: 64 - DNA, 330 bp, Homo sapiens
gatatagtgatgacgcagtctccagccaccctgtctgtgtctccaggggaaagagccaccctctcctgcagggccagtcagagtgttagcagcaacttagcctggtaccaggagagacctggccaggctcccagactcctcatctatggtgcatccaccagggccactggtatcccagccaggttcagtggcagtgggtctgggacagagttcactctcaccatcagcagcctgcagtctgaagattttgcagtttattactgtcagcagtataataagtggcctccgtggacgttcggccaagggaccaaggtggaaatcgaacgaact

SEQ ID NO: 65 - protein, 109 aa, Homo sapiens
DIVMTQSPATLSVSPGERATLSCRASQSVSSNLAWYQERPGQAPRLLIYGASTRATGIPARFSGSGSGTEFTLTISSLQSEDFAVYYCQQYNKWPPWTFGQGTKVEIER

SEQ ID NO: 66 - protein, 23 aa, Homo sapiens
DIVMTQSPATLSVSPGERATLSC

SEQ ID NO: 67 - protein, 11 aa, Homo sapiens
RASQSVSSNLA

SEQ ID NO: 68 - protein, 15 aa, Homo sapiens
WYQERPGQAPRLLIY

SEQ ID NO: 69 - protein, 7 aa, Homo sapiens
GASTRAT

SEQ ID NO: 70 - protein, 32 aa, Homo sapiens; identical to SEQ ID NO: 34

SEQ ID NO: 71 - protein, 10 aa, Homo sapiens
QQYNKWPPWT

SEQ ID NO: 72 - protein, 11 aa, Homo sapiens
FGQGTKVEIER

SEQ ID NO: 73 - DNA, 378 bp, Homo sapiens
cagctggtggagtctgggggaggcgtggtccagcctgggaggtccctgagactctcctgtgcagcctctggattcgccttcagtagctatggcatgcactgggtccgccaggctccaggcaaggggctggagtgggtggcagttatatcatatgatggaaataataaatactatgcagactccgtgaagggccgattcaccatctccagagacaattccaagaacacgctgtatctgcaaatgaacagcctgagagctgaggacacggctgtgtattactgtgcgagagatctagtggttcggggaattagggggtactactactacttcggtatggacgtctggggccaagggaccacggtcaccgtctcctcagcc

SEQ ID NO: 74 - protein, 126 aa, Homo sapiens
QLVESGGGVVQPGRSLRLSCAASGFAFSSYGMHWVRQAPGKGLEWVAVISYDGNNKYYADSVKGRFTISRDNSKNTLYLQMNSLRAEDTAVYYCARDLVVRGIRGYYYYFGMDVWGQGTTVTVSSA

SEQ ID NO: 75 - protein, 23 aa, Homo sapiens
QLVESGGGVVQPGRSLRLSCAAS

SEQ ID NO: 76 - protein, 10 aa, Homo sapiens
GFAFSSYGMH

SEQ ID NO: 77 - protein, 14 aa, Homo sapiens
WVRQAPGKGLEWVA

SEQ ID NO: 78 - protein, 17 aa, Homo sapiens
VISYDGNNKYYADSVKG

SEQ ID NO: 79 - protein, 32 aa, Homo sapiens
RFTISRDNSKNTLYLQMNSLRAEDTAVYYCAR

SEQ ID NO: 80 - protein, 18 aa, Homo sapiens
DLVVRGIRGYYYYFGMDV

SEQ ID NO: 81 - protein, 12 aa, Homo sapiens
WGQGTTVTVSSA

SEQ ID NO: 82 - DNA, 339 bp, Homo sapiens
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaggtctacaaactccgatcaccttcggccaagggacacgactggagattaaacga

SEQ ID NO: 83 - protein, 113 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPITFGQGTRLEIKR

SEQ ID NO: 84 - protein, 23 aa, Homo sapiens
DIVMTQSPLSLPVTPGEPASISC

SEQ ID NO: 85 - protein, 16 aa, Homo sapiens
RSSQSLLHSNGYNYLD

SEQ ID NO: 86 - protein, 15 aa, Homo sapiens
WYLQKPGQSPQLLIY

SEQ ID NO: 87 - protein, 7 aa, Homo sapiens
LGSNRAS

SEQ ID NO: 88 - protein, 32 aa, Homo sapiens
GVPDRFSGSGSGTDFTLKISRVEAEDVGVYYC

SEQ ID NO: 89 - protein, 9 aa, Homo sapiens
MQGLQTPIT

SEQ ID NO: 90 - protein, 11 aa, Homo sapiens
FGQGTRLEIKR

SEQ ID NO: 91 - DNA, 366 bp, Homo sapiens
caggtgcagctggtggagtctgggggaggcgtggtccagcctgggaggtccctgagactctcctgtgcagcgtctggattcaccttcagtaactatggcattcactgggtccgccaggctccaggcaaggggctggagtgggtggcagttatatggtttgatggacgtaataaatactatgcagactccgtgaagggccgattcaccatctccagagacaattccaagaacacgctgtatctgcaaatgaacagcctgagagccgaggacgcggctgtgtattactgtgcgagagatccctttgactatggtgactccttctttgactactggggccagggcaccctggtcaccgtctcctcagcc

SEQ ID NO: 92 - protein, 122 aa, Homo sapiens
QVQLVESGGGVVQPGRSLRLSCAASGFTFSNYGIHWVRQAPGKGLEWVAVIWFDGRNKYYADSVKGRFTISRDNSKNTLYLQMNSLRAEDAAVYYCARDPFDYGDSFFDYWGQGTLVTVSSA

SEQ ID NO: 93 - protein, 25 aa, Homo sapiens
QVQLVESGGGVVQPGRSLRLSCAAS

SEQ ID NO: 94 - protein, 10 aa, Homo sapiens
GFTFSNYGIH

SEQ ID NO: 95 - protein, 14 aa, Homo sapiens
WVRQAPGKGLEWVA

SEQ ID NO: 96 - protein, 17 aa, Homo sapiens
VIWFDGRNKYYADSVKG

SEQ ID NO: 97 - protein, 32 aa, Homo sapiens
RFTISRDNSKNTLYLQMNSLRAEDAAVYYCAR

SEQ ID NO: 98 - protein, 12 aa, Homo sapiens
DPFDYGDSFFDY

SEQ ID NO: 99 - protein, 12 aa, Homo sapiens
WGQGTLVTVSSA

SEQ ID NO: 100 - DNA, 315 bp, Homo sapiens
ctgactcagtctccatcctccctgtctgcatctgtaagagacagagtcaccatcacttgccgggcgagtcaggacattagcaattatttagcctggtatcagcagaaaccagggaaagttcctaatctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccgctcactttcggcggagggaccaaggtggagatcaaacga

SEQ ID NO: 101 - protein, 105 aa, Homo sapiens
LTQSPSSLSASVRDRVTITCRASQDISNYLAWYQQKPGKVPNLLIYAASTLQSGVPSRFSGSGSGTDFTLTISSLQPEDVATYYCQKYNSAPLTFGGGTKVEIKR

SEQ ID NO: 102 - protein, 20 aa, Homo sapiens
LTQSPSSLSASVRDRVTITC

SEQ ID NO: 103 - protein, 11 aa, Homo sapiens
RASQDISNYLA

SEQ ID NO: 104 - protein, 15 aa, Homo sapiens
WYQQKPGKVPNLLIY

SEQ ID NO: 105 - protein, 6 aa, Homo sapiens
AASTLQ

SEQ ID NO: 106 - protein, 32 aa, Homo sapiens
GVPSRFSGSGSGTDFTLTISSLQPEDVATYYC

SEQ ID NO: 107 - protein, 9 aa, Homo sapiens
QKYNSAPLT

SEQ ID NO: 108 - protein, 11 aa, Homo sapiens
FGGGTKVEIKR

SEQ ID NO: 109 - DNA, 366 bp, Homo sapiens
caggtgcagctggtggagtctgggggaggcttggtcaagcctggagggtccctgagactctcctgtgcagcctctggattcaccttcagtgactactacatgacctggatccgccaggctccagggaaggggctggagtgggtttcatacattagtattagtggtagtatcacacactacgcagactcagtgaagggccgattcaccatgtccagggacaacgccaagaactcactgtatctgcaaatgaacagcctgagagccgaggacacggccgtgtattactgtgcgagagacggagcagcagctggtacggatgcttttgatatctggggccacgggacaaaggtcaccgtctcttcagcc

SEQ ID NO: 110 - protein, 122 aa, Homo sapiens
QVQLVESGGGLVKPGGSLRLSCAASGFTFSDYYMTWIRQAPGKGLEWVSYISISGSITHYADSVKGRFTMSRDNAKNSLYLQMNSLRAEDTAVYYCARDGAAAGTDAFDIWGHGTKVTVSSA

SEQ ID NO: 111 - protein, 25 aa, Homo sapiens
QVQLVESGGGLVKPGGSLRLSCAAS

SEQ ID NO: 112 - protein, 10 aa, Homo sapiens
GFTFSDYYMT

SEQ ID NO: 113 - protein, 14 aa, Homo sapiens
WIRQAPGKGLEWVS

SEQ ID NO: 114 - protein, 17 aa, Homo sapiens
YISISGSITHYADSVKG

SEQ ID NO: 115 - protein, 32 aa, Homo sapiens
RFTMSRDNAKNSLYLQMNSLRAEDTAVYYCAR

SEQ ID NO: 116 - protein, 12 aa, Homo sapiens
DGAAAGTDAFDI

SEQ ID NO: 117 - protein, 12 aa, Homo sapiens
WGHGTKVTVSSA

SEQ ID NO: 118 - DNA, 321 bp, Homo sapiens
gagatagtgatgacgcagtctccagccaccctatctgtgtctccaggggacagagccaccctctcctgcagggccagtcagaatgttagcagcaacttggcctggtaccagcagaaacctggccaggctcccaggctcctcatctttggtgcatccaccagggccactggtatcccagccaggttcagtggcagtgggtctgggacagagttcactctcaccatcagcagcctacagtctgaagattttgcagtttattactgtcagcagtatcattactggcccactttcggccctgggaccaaagtggatatcaaacga

SEQ ID NO: 119 - protein, 107 aa, Homo sapiens
EIVMTQSPATLSVSPGDRATLSCRASQNVSSNLAWYQQKPGQAPRLLIFGASTRATGIPARFSGSGSGTEFTLTISSLQSEDFAVYYCQQYHYWPTFGPGTKVDIKR

SEQ ID NO: 120 - protein, 23 aa, Homo sapiens
EIVMTQSPATLSVSPGDRATLSC

SEQ ID NO: 121 - protein, 11 aa, Homo sapiens
RASQNVSSNLA

SEQ ID NO: 122 - protein, 15 aa, Homo sapiens
WYQQKPGQAPRLLIF

SEQ ID NO: 123 - protein, 7 aa, Homo sapiens
GASTRAT

SEQ ID NO: 124 - protein, 32 aa, Homo sapiens; identical to SEQ ID NO: 34

SEQ ID NO: 125 - protein, 8 aa, Homo sapiens
QQYHYWPT

SEQ ID NO: 126 - protein, 11 aa, Homo sapiens
FGPGTKVDIKR

SEQ ID NO: 127 - DNA, 360 bp, Homo sapiens
cagctggtgcagtctggggctgaggtgaagaagcctggggcctcagtgaaggtctcctgcaaggcttctggatacaccttcaccggcttctatatgcactgggtgcgacagacccctggacaagggcttgagtggatgggatggatcaaccctaacagtggtggcacatattatgtacagaagtttcagggcagggtcaccatgaccagggacacgtccatcagcacagtctacatggagctgagcaggttgagatctgacgacacggccgtatattactgtgcgagagatgggtatagcagtggagaggactggttcgacccctggggccagggaaccctggtcaccgtctcctcagcc

SEQ ID NO: 128 - protein, 120 aa, Homo sapiens
QLVQSGAEVKKPGASVKVSCKASGYTFTGFYMHWVRQTPGQGLEWMGWINPNSGGTYYVQKFQGRVTMTRDTSISTVYMELSRLRSDDTAVYYCARDGYSSGEDWFDPWGQGTLVTVSSA

SEQ ID NO: 129 - protein, 23 aa, Homo sapiens
QLVQSGAEVKKPGASVKVSCKAS

SEQ ID NO: 130 - protein, 10 aa, Homo sapiens
GYTFTGFYMH

SEQ ID NO: 131 - protein, 14 aa, Homo sapiens
WVRQTPGQGLEWMG

SEQ ID NO: 132 - protein, 17 aa, Homo sapiens
WINPNSGGTYYVQKFQG

SEQ ID NO: 133 - protein, 32 aa, Homo sapiens
RVTMTRDTSISTVYMELSRLRSDDTAVYYCAR

SEQ ID NO: 134 - protein, 12 aa, Homo sapiens
DGYSSGEDWFDP

SEQ ID NO: 135 - protein, 12 aa, Homo sapiens
WGQGTLVTVSSA

SEQ ID NO: 136 - DNA, 339 bp, Homo sapiens
gatattgtgatgacccagactccactctctctgtccgtcacccctggacagccggcctccatctcctgcaagtctagtcagagcctcctgcatagtggtggaaagacctatttgtattggtacctgcagaggccaggccagcctccacagctcctgatctatgaagtttccaaccggttctctggagtgccagataggttcagtggcagcgggtcagggacagatttcacactgaaaatcagccgggtggaggctgaggatgttggggtttattactgcatgcaaagtatacaccttccgctcactttcggcggagggaccaaggtggagatcaaacga

SEQ ID NO: 137 - protein, 113 aa, Homo sapiens
DIVMTQTPLSLSVTPGQPASISCKSSQSLLHSGGKTYLYWYLQRPGQPPQLLIYEVSNRFSGVPDRFSGSGSGTDFTLKISRVEAEDVGVYYCMQSIHLPLTFGGGTKVEIKR

SEQ ID NO: 138 - protein, 23 aa, Homo sapiens
DIVMTQTPLSLSVTPGQPASISC

SEQ ID NO: 139 - protein, 16 aa, Homo sapiens
KSSQSLLHSGGKTYLY

SEQ ID NO: 140 - protein, 15 aa, Homo sapiens
WYLQRPGQPPQLLIY

SEQ ID NO: 141 - protein, 7 aa, Homo sapiens
EVSNRFS

SEQ ID NO: 142 - protein, 32 aa, Homo sapiens; identical to SEQ ID NO: 88

SEQ ID NO: 143 - protein, 9 aa, Homo sapiens
MQSIHLPLT

SEQ ID NO: 144 - protein, 11 aa, Homo sapiens
FGGGTKVEIKR

SEQ ID NO: 145 - DNA, 360 bp, Homo sapiens
caggtgcagctggagcagtcggggggaggcctggtcaagcctggggggtccctgagattctcctgtgcagcctctggattcaccttcagtagctatagcatgaactgggtccgccaggctccagggaaggggctggagtgggtctcattcattagtagtagtagtagttacatatactacgcagactcagtgaagggccgattcaccatctccagagacaacgccaagaactcactgtatctgcaaatgaacagcctgagagccgaggacacggctgtgtattactgtgcgagagaggactgggtgggagctacctttgactactggggccagggaaccctggtcaccgtctcctcagcc

SEQ ID NO: 146 - protein, 120 aa, Homo sapiens
QVQLEQSGGGLVKPGGSLRFSCAASGFTFSSYSMNWVRQAPGKGLEWVSFISSSSSYIYYADSVKGRFTISRDNAKNSLYLQMNSLRAEDTAVYYCAREDWVGATFDYWGQGTLVTVSSA

SEQ ID NO: 147 - protein, 25 aa, Homo sapiens
QVQLEQSGGGLVKPGGSLRFSCAAS

SEQ ID NO: 148 - protein, 10 aa, Homo sapiens
GFTFSSYSMN

SEQ ID NO: 149 - protein, 14 aa, Homo sapiens
WVRQAPGKGLEWVS

SEQ ID NO: 150 - protein, 17 aa, Homo sapiens
FISSSSSYIYYADSVKG

SEQ ID NO: 151 - protein, 32 aa, Homo sapiens
RFTISRDNAKNSLYLQMNSLRAEDTAVYYCAR

SEQ ID NO: 152 - protein, 10 aa, Homo sapiens
EDWVGATFDY

SEQ ID NO: 153 - protein, 12 aa, Homo sapiens
WGQGTLVTVSSA

SEQ ID NO: 154 - DNA, 324 bp, Homo sapiens
gacattcagctgacccagtctccatcctccctgtctgcatctgtaggagacagagtcaccatcacttgtcgggcgagtcagggcattaggaattatttagcctggtatcagcagaaaccagggaaagttcctaagctcctgatctatgctgcttccgctttgaaattaggggtcccatctcggttcagtggcagtggatctgggacagatttcactctcaccatcagcagcctgcagcctgaagatgttgcaacttattactgtcaaaagtataacagtgccccgatcaccttcggccaagggacacgactggacattaaacga

SEQ ID NO: 155 - protein, 108 aa, Homo sapiens
DIQLTQSPSSLSASVGDRVTITCRASQGIRNYLAWYQQKPGKVPKLLIYAASALKLGVPSRFSGSGSGTDFTLTISSLQPEDVATYYCQKYNSAPITFGQGTRLDIKR

SEQ ID NO: 156 - protein, 23 aa, Homo sapiens
DIQLTQSPSSLSASVGDRVTITC

SEQ ID NO: 157 - protein, 11 aa, Homo sapiens
RASQGIRNYLA

SEQ ID NO: 158 - protein, 15 aa, Homo sapiens
WYQQKPGKVPKLLIY

SEQ ID NO: 159 - protein, 7 aa, Homo sapiens
AASALKL

SEQ ID NO: 160 - protein, 32 aa, Homo sapiens; identical to SEQ ID NO: 106

SEQ ID NO: 161 - protein, 9 aa, Homo sapiens
QKYNSAPIT

SEQ ID NO: 162 - protein, 11 aa, Homo sapiens
FGQGTRLDIKR

SEQ ID NO: 163 - DNA, 396 bp, Homo sapiens
caggtgcagctggagcagtcgggcccaggactggtgaagccttcacagaacctgtccctcacctgcactgtctctggtggctccatcagcagtggtggttatttctggagctggatccgccagcacccagggaagggcctggagtggattgggtacatctattacagtgggaacacctactacaacccgtccctcaagagtcgagttaccatatcagttgacacgtctaagaaccagttctccctgaaactgagctctgtgactgccgcggacacggccgtgtattactgtgcgagagactattactatgatactagtggtttttcctaccgttacgactggtactacggtatggacgtctggggccaagggaccacggtcaccgtctcctcagcc

SEQ ID NO: 164 - protein, 132 aa, Homo sapiens
QVQLEQSGPGLVKPSQNLSLTCTVSGGSISSGGYFWSWIRQHPGKGLEWIGYIYYSGNTYYNPSLKSRVTISVDTSKNQFSLKLSSVTAADTAVYYCARDYYYDTSGFSYRYDWYYGMDVWGQGTTVTVSSA

SEQ ID NO: 165 - protein, 25 aa, Homo sapiens
QVQLEQSGPGLVKPSQNLSLTCTVS

SEQ ID NO: 166 - protein, 12 aa, Homo sapiens
GGSISSGGYFWS

SEQ ID NO: 167 - protein, 14 aa, Homo sapiens
WIRQHPGKGLEWIG

SEQ ID NO: 168 - protein, 16 aa, Homo sapiens
YIYYSGNTYYNPSLKS

SEQ ID NO: 169 - protein, 32 aa, Homo sapiens; identical to SEQ ID NO: 7

SEQ ID NO: 170 - protein, 21 aa, Homo sapiens
DYYYDTSGFSYRYDWYYGMDV

SEQ ID NO: 171 - protein, 12 aa, Homo sapiens
WGQGTTVTVSSA

SEQ ID NO: 172 - DNA, 321 bp, Homo sapiens
gacatccagctgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaaatggggtcccatcaaggttcagcggcagtggatctgggacagaattcactctcacaatcagcagcctgcagcctgaagattttgcaacttattactgtctacaacataatacttacccggcgttcggccaagggaccaaggtggaaatcaaacga

SEQ ID NO: 173 - protein, 107 aa, Homo sapiens
DIQLTQSPSSLSASVGDRVTITCRASQGIRNDLGWYQQKPGKAPKRLIYAASSLQNGVPSRFSGSGSGTEFTLTISSLQPEDFATYYCLQHNTYPAFGQGTKVEIKR

SEQ ID NO: 174 - protein, 23 aa, Homo sapiens
DIQLTQSPSSLSASVGDRVTITC

SEQ ID NO: 175 - protein, 11 aa, Homo sapiens
RASQGIRNDLG

SEQ ID NO: 176 - protein, 15 aa, Homo sapiens
WYQQKPGKAPKRLIY

SEQ ID NO: 177 - protein, 7 aa, Homo sapiens
AASSLQN

SEQ ID NO: 178 - protein, 32 aa, Homo sapiens
GVPSRFSGSGSGTEFTLTISSLQPEDFATYYC

SEQ ID NO: 179 - protein, 8 aa, Homo sapiens
LQHNTYPA

SEQ ID NO: 180 - protein, 11 aa, Homo sapiens
FGQGTKVEIKR

SEQ ID NO: 181 - DNA, 354 bp, Homo sapiens
cagctggtgcagtctggagcagaagtgaaaaagcccggggagtctctgaagatctcctgtcagggttctggatacatctttaccaactactggatcggctgggtgcgccagatgcccgggaaaggcctggagtggatgggggtcatctatcctgatgactctgataccagatacagcccgtccttccaaggccaggtcaccatctcagccgacaagtccatcagcaccgcctacctgcagtggagcagcctgaaggcctcggacaccgccatatattactgtgcgagacaaaaatggctacaacacccctttgactactggggccagggaaccctggtcaccgtctcctcagcc

SEQ ID NO: 182 - protein, 118 aa, Homo sapiens
QLVQSGAEVKKPGESLKISCQGSGYIFTNYWIGWVRQMPGKGLEWMGVIYPDDSDTRYSPSFQGQVTISADKSISTAYLQWSSLKASDTAIYYCARQKWLQHPFDYWGQGTLVTVSSA

SEQ ID NO: 183 - protein, 23 aa, Homo sapiens
QLVQSGAEVKKPGESLKISCQGS

SEQ ID NO: 184 - protein, 10 aa, Homo sapiens
GYIFTNYWIG

SEQ ID NO: 185 - protein, 14 aa, Homo sapiens
WVRQMPGKGLEWMG

SEQ ID NO: 186 - protein, 17 aa, Homo sapiens
VIYPDDSDTRYSPSFQG

SEQ ID NO: 187 - protein, 32 aa, Homo sapiens
QVTISADKSISTAYLQWSSLKASDTAIYYCAR

SEQ ID NO: 188 - protein, 10 aa, Homo sapiens
QKWLQHPFDY

SEQ ID NO: 189 - protein, 12 aa, Homo sapiens
WGQGTLVTVSSA

SEQ ID NO: 190 - DNA, 327 bp, Homo sapiens
gaaattgtgttgacgcagtcaccaggcaccctgtctttgtctccaggggaaagagtcaccctctcatgcagggccagtcagagtgttagcagcagatacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtttattactgtcagcagtatggtagctcacctcggacgttcggccaagggaccaaggtggaaatcaaacga

SEQ ID NO: 191 - protein, 109 aa, Homo sapiens
EIVLTQSPGTLSLSPGERVTLSCRASQSVSSRYLAWYQQKPGQAPRLLIYGASSRATGIPDRFSGSGSGTDFTLTISRLEPEDFAVYYCQQYGSSPRTFGQGTKVEIKR

SEQ ID NO: 192 - protein, 23 aa, Homo sapiens
EIVLTQSPGTLSLSPGERVTLSC

SEQ ID NO: 193 - protein, 12 aa, Homo sapiens
RASQSVSSRYLA

SEQ ID NO: 194 - protein, 15 aa, Homo sapiens
WYQQKPGQAPRLLIY

SEQ ID NO: 195 - protein, 7 aa, Homo sapiens
GASSRAT

SEQ ID NO: 196 - protein, 31 aa, Homo sapiens
GIPDRFSGSGSGTDFTLTISRLEPEDFAVYY

SEQ ID NO: 197 - protein, 9 aa, Homo sapiens
QQYGSSPRT

SEQ ID NO: 198 - protein, 11 aa, Homo sapiens
FGQGTKVEIKR

SEQ ID NO: 199 - DNA, 369 bp, Homo sapiens
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcaggacagggtcaccatgaccagggacacgtccatcagcacagcctacatggagctgagcaggctgagatctgacgacacggccgtgtattactgtgcgagagatttctttggttcggggagtctcctctactttgactactggggccagggaaccctggtcaccgtctcctcagcc

SEQ ID NO: 200 - protein, 123 aa, Homo sapiens
QVQLVQSGAEVKKPGASVKVSCKASGYTFTGYYMHWVRQAPGQGLEWMGWINPNSGGTNYAQKFQDRVTMTRDTSISTAYMELSRLRSDDTAVYYCARDFFGSGSLLYFDYWGQGTLVTVSSA

SEQ ID NO: 201 - protein, 25 aa, Homo sapiens [sequence omitted]

SEQ ID NO: 202 - protein, 10 aa, Homo sapiens
GYTFTGYYMH

SEQ ID NO: 203 - protein, 14 aa, Homo sapiens
WVRQAPGQGLEWMG

SEQ ID NO: 204 - protein, 17 aa, Homo sapiens
WINPNSGGTNYAQKFQD

SEQ ID NO: 205 - protein, 32 aa, Homo sapiens
RVTMTRDTSISTAYMELSRLRSDDTAVYYCAR

SEQ ID NO: 206 - protein, 13 aa, Homo sapiens
DFFGSGSLLYFDY

SEQ ID NO: 207 - protein, 12 aa, Homo sapiens
WGQGTLVTVSSA

SEQ ID NO: 208 - DNA, 339 bp, Homo sapiens; identical to SEQ ID NO: 136

SEQ ID NO: 209 - protein, 113 aa, Homo sapiens; identical to SEQ ID NO: 137

SEQ ID NO: 210 - protein, 23 aa, Homo sapiens
DIVMTQTPLSLSVTPGQPASISC

SEQ ID NO: 211 - protein, 16 aa, Homo sapiens
KSSQSLLHSGGKTYLY

SEQ ID NO: 212 - protein, 15 aa, Homo sapiens
WYLQRPGQPPQLLIY

SEQ ID NO: 213 - protein, 7 aa, Homo sapiens
EVSNRFS

SEQ ID NO: 214 - protein, 32 aa, Homo sapiens; identical to SEQ ID NO: 88

SEQ ID NO: 215 - protein, 9 aa, Homo sapiens
MQSIHLPLT

SEQ ID NO: 216 - protein, 11 aa, Homo sapiens
FGGGTKVEIKR

SEQ ID NO: 217 - DNA, 366 bp, Homo sapiens
caggtgcagctggagcagtcggggggaggcgtggtccagcctgggaggtccctgagactctcctgtgcagcgtctggattcaccttcaataactatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgaaagatgaggaatactactatgtttcggggcttgactactggggccagggaaccctggtcaccgtctcctcagcc

SEQ ID NO: 218 - protein, 122 aa, Homo sapiens
QVQLEQSGGGVVQPGRSLRLSCAASGFTFNNYGMHWVRQAPGKGLEWVAVIWYDGSNKYYADSVKGRFTISRDNSKNTLYLQMNSLRAEDTAVYYCAKDEEYYYVSGLDYWGQGTLVTVSSA

SEQ ID NO: 219 - protein, 25 aa, Homo sapiens
QVQLEQSGGGVVQPGRSLRLSCAAS

SEQ ID NO: 220 - protein, 10 aa, Homo sapiens
GFTFNNYGMH

SEQ ID NO: 221 - protein, 14 aa, Homo sapiens
WVRQAPGKGLEWVA

SEQ ID NO: 222 - protein, 17 aa, Homo sapiens
VIWYDGSNKYYADSVKG

SEQ ID NO: 223 - protein, 32 aa, Homo sapiens
RFTISRDNSKNTLYLQMNSLRAEDTAVYYCAK

SEQ ID NO: 224 - protein, 12 aa, Homo sapiens
DEEYYYVSGLDY

SEQ ID NO: 225 - protein, 12 aa, Homo sapiens
WGQGTLVTVSSA

SEQ ID NO: 226 - DNA, 315 bp, Homo sapiens; identical to SEQ ID NO: 100

SEQ ID NO: 227 - protein, 105 aa, Homo sapiens; identical to SEQ ID NO: 101

SEQ ID NO: 228 - protein, 20 aa, Homo sapiens
LTQSPSSLSASVRDRVTITC

SEQ ID NO: 229 - protein, 11 aa, Homo sapiens
RASQDISNYLA

SEQ ID NO: 230 - protein, 15 aa, Homo sapiens
WYQQKPGKVPNLLIY

SEQ ID NO: 231 - protein, 6 aa, Homo sapiens
AASTLQ

SEQ ID NO: 232 - protein, 32 aa, Homo sapiens; identical to SEQ ID NO: 106

SEQ ID NO: 233 - protein, 9 aa, Homo sapiens
QKYNSAPLT

SEQ ID NO: 234 - protein, 11 aa, Homo sapiens
FGGGTKVEIKR

SEQ ID NO: 235 - DNA, 372 bp, Homo sapiens
cagatcaccttgaaggagtctggtcctacgctggtgacacccacacagaccctcacgctgacctgcaccttctctgggttctcactcagcactggtggaatgggtgtgggctggatccgtcagcccccaggaaaggccctggactggcttacactcatttattggaatgatgataagcactacagcccatctctgaagagcaggcttaccatcaccaaggacacctccaaaaaccaggtggtccttagaatgaccaacatggaccctgtggacacagccacttattactgtgcacacctgcattacgatattttgactggttttaactttgactactggggccagggaaccctggtcaccgtctcctcagcc

SEQ ID NO: 236 - protein, 124 aa, Homo sapiens
QITLKESGPTLVTPTQTLTLTCTFSGFSLSTGGMGVGWIRQPPGKALDWLTLIYWNDDKHYSPSLKSRLTITKDTSKNQVVLRMTNMDPVDTATYYCAHLHYDILTGFNFDYWGQGTLVTVSSA

SEQ ID NO: 237 - protein, 25 aa, Homo sapiens
QITLKESGPTLVTPTQTLTLTCTFS

SEQ ID NO: 238 - protein, 12 aa, Homo sapiens
GFSLSTGGMGVG

SEQ ID NO: 239 - protein, 14 aa, Homo sapiens
WIRQPPGKALDWLT

SEQ ID NO: 240 - protein, 16 aa, Homo sapiens
LIYWNDDKHYSPSLKS

SEQ ID NO: 241 - protein, 32 aa, Homo sapiens
RLTITKDTSKNQVVLRMTNMDPVDTATYYCAH

SEQ ID NO: 242 - protein, 13 aa, Homo sapiens
LHYDILTGFNFDY

SEQ ID NO: 243 - protein, 12 aa, Homo sapiens
WGQGTLVTVSSA

SEQ ID NO: 244 - DNA, 342 bp, Homo sapiens
gatattgtgatgacccagactccactctccctgcccgtcacccctggagagccggcctccatctcctgcaggtctagtcagagcctcttggatagtgatgatggaaacacctatttggactggtacctgcagaagccagggcagtctccacagctcctgatctatacgctttcctatcgggcctctggagtcccagacaggttcagtggcagtgggtcaggcactgatttcacactgaaaatcagcagggtggaggctgaggatgttggagtttattactgcatgcaacgtatagagtttccgctcactttcggcggagggaccaaggtggagatcaaacga

SEQ ID NO: 245 - protein, 114 aa, Homo sapiens
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPLTFGGGTKVEIKR

SEQ ID NO: 246 - protein, 23 aa, Homo sapiens
DIVMTQTPLSLPVTPGEPASISC

SEQ ID NO: 247 - protein, 17 aa, Homo sapiens
RSSQSLLDSDDGNTYLD

SEQ ID NO: 248 - protein, 15 aa, Homo sapiens
WYLQKPGQSPQLLIY

SEQ ID NO: 249 - protein, 7 aa, Homo sapiens
TLSYRAS

SEQ ID NO: 250 - protein, 32 aa, Homo sapiens; identical to SEQ ID NO: 88

SEQ ID NO: 251 - protein, 9 aa, Homo sapiens
MQRIEFPLT

SEQ ID NO: 252 - protein, 11 aa, Homo sapiens
FGGGTKVEIKR

SEQ ID NO: 253 - protein, 94 aa, Homo sapiens
  VARIANT (1)..(1): Xaa is E or Q
XSGPGLVKPSQXLSLTCTVSGGSISSXXYXWXWIRXHPGKGLEWIGYIYYSGXTYXNPSLKSRVXISVDTSKNQFSLXLSSVTAADTAVYYCAR

SEQ ID NO: 254 - protein, 12 aa, Homo sapiens
  VARIANT (7)..(7): Xaa is A, F, or G
GGSISSXXYXWX

SEQ ID NO: 255 - protein, 16 aa, Homo sapiens
  VARIANT (7)..(7): Xaa is S or N
YIYYSGXTYXNPSLKS

SEQ ID NO: 256 - protein, 96 aa, Homo sapiens
  VARIANT (30)..(30): Xaa is Y or F
QLVQSGAEVKKPGASVKVSCKASGYTFTGXYMHWVRQXPGQGLEWMGWINPNSGGTXYXQKFQXRVTMTRDTSISTXYMELSRLRSDDTAVYYCAR

SEQ ID NO: 257 - protein, 10 aa, Homo sapiens
  VARIANT (7)..(7): Xaa is Y or F
GYTFTGXYMH

SEQ ID NO: 258 - protein, 17 aa, Homo sapiens
  VARIANT (10)..(10): Xaa is N or Y
WINPNSGGTXYXQKFQX

SEQ ID NO: 259 - protein, 9 aa, Homo sapiens
  VARIANT (1)..(1): Xaa is Y or D
XXXGSGSXX

SEQ ID NO: 260 - protein, 98 aa, Homo sapiens
  VARIANT (12)..(12): Xaa is K or T
ITLKESGPTLVXPTQTLTLTCTFSGFSLSXXGXGVGWIRQPPGKALXWLXLIYWNDDKXYSPSLXSRLTITKDTSKNQVVLXXTNMDPVDTATYYCAH

SEQ ID NO: 261 - protein, 12 aa, Homo sapiens
  VARIANT (6)..(6): Xaa is T or A
GFSLSXXGXGVG

SEQ ID NO: 262 - protein, 16 aa, Homo sapiens
  VARIANT (9)..(9): Xaa is R or H
LIYWNDDKXYSPSLXS

SEQ ID NO: 263 - protein, 9 aa, Homo sapiens
  VARIANT (1)..(1): Xaa is Y or H
XYDILTGXX

SEQ ID NO: 264 - protein, 7 aa, Homo sapiens
  VARIANT (4)..(4): Xaa is V or D
YDYXWGS

SEQ ID NO: 265 - protein, 98 aa, Homo sapiens
  VARIANT (5)..(5): Xaa is V or E
QVQLXXSGGGVVQPGRSLRLSCAASGFTFXXYGXHWVRQAPGKGLEWVAVIWXDGXNKYYADSVKGRFTISRDNSKNTLYLQMNSLRAEDXAVYYCAX

SEQ ID NO: 266 - protein, 10 aa, Homo sapiens
  VARIANT (5)..(5): Xaa is S or N
GFTFXXYGXH

SEQ ID NO: 267 - protein, 17 aa, Homo sapiens
  VARIANT (4)..(4): Xaa is Y or F
VIWXDGXNKYYADSVKG

SEQ ID NO: 268 - protein, 7 aa, Homo sapiens
  VARIANT (7)..(7): Xaa is S or L
YYYGSGX

SEQ ID NO: 269 - protein, 5 aa, Homo sapiens
  VARIANT (5)..(5): Xaa is Y or S
DYGDX

SEQ ID NO: 270 - protein, 98 aa, Homo sapiens
  VARIANT (31)..(31): Xaa is D or S
QVQLVESGGGLVKPGGSLRLSCAASGFTFSXYXMXWIRQAPGKGLEWVSYISXSGSXXXYADSVKGRFTXSRDNAKNSLYLQMNSLRAEDTAVYYCAR

SEQ ID NO: 271 - protein, 10 aa, Homo sapiens
  VARIANT (6)..(6): Xaa is D or S
GFTFSXYXMX

SEQ ID NO: 272 - protein, 17 aa, Homo sapiens
  VARIANT (4)..(4): Xaa is S or I
YISXSGSXXXYADSVKG

SEQ ID NO: 273 - protein, 10 aa, Homo sapiens
  VARIANT (1)..(1): Xaa is G or D
XXAAAGAFDI

SEQ ID NO: 274 - protein, 98 aa, Homo sapiens
  VARIANT (1)..(1): Xaa is E or Q
XVQLXXSGGGLVKPGGSLRXSCAASGFTFSSYSMNWVRQAPGKGLEWVSXISSSSSYIYYADSVKGRFTISRDNAKNSLYLQMNSLRAEDTAVYYCAR

SEQ ID NO: 275 - protein, 17 aa, Homo sapiens
  VARIANT (1)..(1): Xaa is S or F
XISSSSSYIYYADSVKG

SEQ ID NO: 276 - protein, 9 aa, Homo sapiens
  VARIANT (1)..(1): Xaa is G or D
XXVGATFDY

SEQ ID NO: 277 - protein, 96 aa, Homo sapiens
  VARIANT (26)..(26): Xaa is T or A
QLVESGGGVVQPGRSLRLSCAASGFXFSSYGMHWVRQAPGKGLEWVAVISYDGXNKYYADSVKGRFTISRDNSKNTLYLQMNSLRAEDTAVYYCAK

SEQ ID NO: 278 - protein, 10 aa, Homo sapiens
  VARIANT (3)..(3): Xaa is T or A
GFXFSSYGMH

SEQ ID NO: 279 - protein, 17 aa, Homo sapiens
  VARIANT (7)..(7): Xaa is S or N
VISYDGXNKYYADSVKG

SEQ ID NO: 280 - protein, 9 aa, Homo sapiens
  VARIANT (1)..(1): Xaa is I or D
XXXVRGXXX

SEQ ID NO: 281 - protein, 97 aa, Homo sapiens
  VARIANT (27)..(27): Xaa is G or D
QVQLQESGPGLVKPSETLSLTCTVSGXSISXYYWSWIRQPPGKGLEWIGYXYYSGSTNYNPSLKSRVTISVDTSKNQFSLKLSSVTAADTAVYYCAR

SEQ ID NO: 282 - protein, 10 aa, Homo sapiens
  VARIANT (2)..(2): Xaa is G or D
GXSISXYYWS

SEQ ID NO: 283 - protein, 16 aa, Homo sapiens
  VARIANT (2)..(2): Xaa is I or F
YXYYSGSTNYNPSLKS

SEQ ID NO: 284 - protein, 6 aa, Homo sapiens
  VARIANT (1)..(1): Xaa is S or D
XXGWDY

SEQ ID NO: 285 - protein, 96 aa, Homo sapiens
  VARIANT (21)..(21): Xaa is K or Q
QLVQSGAEVKKPGESLKISCXGSGYXFTXYWIGWVRQMPGKGLEWMGXIYPXDSDTRYSPSFQGQVTISADKSISTAYLQWSSLKASDTAXYYCAR

SEQ ID NO: 286 - protein, 10 aa, Homo sapiens
  VARIANT (3)..(3): Xaa is S or I
GYXFTXYWIG

SEQ ID NO: 287 - protein, 17 aa, Homo sapiens
  VARIANT (1)..(1): Xaa is I or V
XIYPXDSDTRYSPSFQG

SEQ ID NO: 288 - protein, 8 aa, Homo sapiens
  VARIANT (1)..(1): Xaa is R or K
XWLQXFDY

SEQ ID NO: 289 - protein, 470 aa, Homo sapiens
KRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPAS

SEQ ID NO: 290 - protein, 445 aa, Homo sapiens
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 291 - protein, 449 aa, Homo sapiens
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 292 - protein, 12 aa, Homo sapiens
ATTLKSYDSNTP

SEQ ID NO: 293 - protein, 98 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 294 - protein, 100 aa, Homo sapiens
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHR

SEQ ID NO: 295 - protein, 98 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 296 - protein, 98 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 297 - protein, 98 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAK

SEQ ID NO: 298 - protein, 98 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 299 - protein, 99 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCAR

SEQ ID NO: 300 - protein, 97 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAR

SEQ ID NO: 301 - protein, 98 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 302 - DNA, 17 bp, Homo sapiens
ggtataactggaacgac

SEQ ID NO: 303 - DNA, 20 bp, Homo sapiens
ggtatagtgggagctactac

SEQ ID NO: 304 - DNA, 31 bp, Homo sapiens
gtattactatggttcggggagttattataac

SEQ ID NO: 305 - DNA, 37 bp, Homo sapiens
gtattatgattacgtttgggggagttatcgttatacc

SEQ ID NO: 306 - DNA, 31 bp, Homo sapiens
gtattactatgatagtagtggttattactac

SEQ ID NO: 307 - DNA, 31 bp, Homo sapiens
gtattacgatattttgactggttattataac

SEQ ID NO: 308 - DNA, 16 bp, Homo sapiens
tgactacggtgactac

SEQ ID NO: 309 - DNA, 20 bp, Homo sapiens
gtagagatggctacaattac

SEQ ID NO: 310 - DNA, 21 bp, Homo sapiens
gggtatagcagcagctggtac

SEQ ID NO: 311 - DNA, 21 bp, Homo sapiens
gggtatagcagtggctggtac

SEQ ID NO: 312 - protein, 15 aa, Homo sapiens
AFDIWGQGTMVTVSS

SEQ ID NO: 313 - protein, 15 aa, Homo sapiens
YFDYWGQGTLVTVSS

SEQ ID NO: 314 - protein, 16 aa, Homo sapiens
NWFDPWGQGTLVTVSS

SEQ ID NO: 315 - protein, 20 aa, Homo sapiens
YYYYYGMDVWGQGTTVTVSS

SEQ ID NO: 316 - protein, 100 aa, Homo sapiens
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQSIQLP

SEQ ID NO: 317 - protein, 100 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTP

SEQ ID NO: 318 - protein, 95 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAP

SEQ ID NO: 319 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 320 - protein, 95 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYP

SEQ ID NO: 321 - protein, 95 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 322 - protein, 101 aa, Homo sapiens
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFP

SEQ ID NO: 323 - protein, 12 aa, Homo sapiens
WTFGQGTKVEIK

SEQ ID NO: 324 - protein, 12 aa, Homo sapiens
YTFGQGTKLEIK

SEQ ID NO: 325 - protein, 12 aa, Homo sapiens
FTFGPGTKVDIK

SEQ ID NO: 326 - protein, 12 aa, Homo sapiens
LTFGGGTKVEIK

SEQ ID NO: 327 - protein, 12 aa, Homo sapiens
ITFGQGTRLEIK

(19) United States
(12) Patent Application Publication
(10) Pub. No.: US 2014/0314780 A1
(43) Pub. Date: Oct. 23, 2014

(54) Compositions Using Antibodies Directed to GPNMB and Uses Thereof

(71) Applicant: Celldex Therapeutics, Inc., Hampton, NJ (US)

(72) Inventors: Michael E. Jeffers, Branford, CT (US); William J. LaRochelle, Madison, CT (US)

(21) Appl. No.: 14/321,102

(22) Filed: Jul. 1, 2014

Related U.S. Application Data
(63) Continuation of application No. 13/544,526, filed on Jul. 9, 2012, now abandoned.
Application No. 13/544,526 is a continuation of application No. 13/303,264, filed on Nov. 23, 2011, now abandoned.
Application No. 13/303,264 is a continuation of application No. 13/084,139, filed on Apr. 11, 2011, now abandoned.
Application No. 13/084,139 is a continuation of application No. 12/855,162, filed on Aug. 12, 2010, now abandoned.
Application No. 12/855,162 is a continuation of application No. 12/643,421, filed on Dec. 21, 2009, now abandoned.
Application No. 12/643,421 is a continuation of application No. 12/463,178, filed on May 8, 2009, now abandoned.
Application No. 12/463,178 is a continuation of application No. 12/229,184, filed on Aug. 20, 2008, now abandoned.

(51) Int. Cl. A61K 39/395; A61K 45/06; A61K 47/48

(52) U.S. Cl. CPC A61K 39/39558; A61K 47/48569; A61K 45/06
USPC 424/142.1; 424/156.1; 424/178.1; 435/375

(57) ABSTRACT

The present invention relates to antibodies, including fully human monoclonal antibodies, with specificity to GPNMB, and uses of such antibodies. The present invention further provides compositions that increase expression of GPNMB on the surface of tumor cells, and methods of using such compositions to increase the anti-cancer activity or other therapeutic efficacy of the antibodies and immunoconjugates provided herein.

### RELATED APPLICATIONS

This application is a continuation of U.S. patent application Ser. No. 13/544,526, filed Jul. 9, 2012, which is a continuation of U.S. patent application Ser. No. 13/303,264, filed Nov. 23, 2011, which is a continuation of U.S. patent application Ser. No. 13/084,139, filed Apr. 11, 2011, which is a continuation of U.S. patent application Ser. No. 12/855,162, filed Aug. 12, 2010, which is a continuation of U.S. patent application Ser. No. 12/643,421, filed Dec. 21, 2009, which is a continuation of U.S. patent application Ser. No. 12/463,178, filed May 8, 2009, which is a continuation of U.S. patent application Ser. No. 12/229,184, filed Aug. 20, 2008, the contents of each of which are hereby incorporated by reference in their entirety.

### INCORPORATION BY REFERENCE OF SEQUENCE LISTING

The contents of the text file named “698C05USSeqList.txt”, which was created on Nov. 23, 2011 and is 132 KB in size, are hereby incorporated by reference in their entirety.

### FIELD OF THE INVENTION

The present invention relates to antibodies, including fully human monoclonal antibodies, with specificity to GPNMB, and uses of such antibodies. The present invention further provides compositions that increase expression of GPNMB on the surface of tumor cells, and methods of using such compositions to increase the anti-cancer activity or other therapeutic efficacy of the antibodies and immunoconjugates provided herein.

### BACKGROUND OF THE INVENTION

A putative transmembrane glycoprotein called “nmb” (Acc. No. X76534 EMBL), referred to herein as GPNMB, has been identified as being differentially expressed in metastatic human melanoma cancer cell lines and xenografts.

It would be desirable to have an antibody suitable for in vivo targeting of GPNMB expressing pathologies and to enable therapeutic efficacy. It would also be desirable to have antibody and/or immunoconjugate compositions that increase the anti-cancer activity or other therapeutic efficacy of these composition for use in treating GPNMB-expressing pathologies.

### SUMMARY OF THE INVENTION

The current invention provides human monoclonal antibodies that specifically bind GPNMB as well as variants, derivatives and antigen binding fragments of such antibodies. The present invention further provides compositions that increase expression of GPNMB on the surface of tumor cells, and methods of using such compositions to increase the anti-cancer activity or other therapeutic efficacy of the antibodies and immunoconjugates provided herein.

The invention provides pharmaceutical compositions that include an isolated monoclonal antibody that specifically binds to GPNMB and a second agent that increases expression of GPNMB on a tumor cell or decreases shedding of GPNMB by a tumor cell. In some embodiments, the second agent is selected from an inhibitor of the ERK pathway, a tyrosine kinase inhibitor, an inhibitor of p38 MAPK, a lysosomotropic weak base and an inhibitor of GPNMB shedding. In some embodiments, the antibody is a human monoclonal antibody.

In some embodiments, the antibody includes a heavy chain variable region comprising an amino acid sequence selected from the group consisting of SEQ ID NO: 2, 20, 38, 56, 74, 92, 110, 128, 146, 164, 182, 200, 218, 236, 253, 256, 260, 265, 270, 274, 277, 281 and 285; and the antibody includes a light chain variable region comprising an amino acid sequence selected from the group consisting of: SEQ ID NO: 11, 29, 47, 65, 83, 101, 119, 137, 155, 173, 191, 209, 227 and 245.

In some embodiments, the antibody includes (a) a V_{H} CDR1 region comprising the amino acid sequence of SEQ ID NO: 4, 22, 40, 58, 76, 94, 112, 130, 148, 166, 184, 202, 220, 238, 254, 257, 261, 266, 271, 278, 282 or 286; (b) a V_{H} CDR2 region comprising the amino acid sequence of SEQ ID NO: 6, 24, 42, 60, 78, 96, 114, 132, 150, 168, 186, 204, 222, 240, 255, 258, 262, 267, 272, 275, 279, 283 or 287; (c) a V_{H} CDR3 region comprising the amino acid sequence of SEQ ID NO: 8, 26, 44, 62, 80, 98, 116, 134, 152, 170, 188, 206, 224, 242, 259, 263, 264, 268, 269, 273, 276, 280, 284 or 288; (d) a V_{L} CDR1 region comprising the amino acid sequence of SEQ ID NO: 13, 31, 49, 67, 85, 103, 121, 139, 157, 175, 193, 211, 229 or 247; (e) a V_{L} CDR2 region comprising the amino acid sequence of SEQ ID NO: 15, 33, 51, 69, 87, 105, 123, 141, 159, 177, 195, 213, 231, 249 or 279; and (f) a V_{L} CDR3 region comprising the amino acid sequence of SEQ ID NO: 17, 35, 53, 71, 89, 107, 125, 143, 161, 179, 197, 215, 233 or 251. In some embodiments, the antibody is an IgG1 antibody.

In some embodiments, the antibody is conjugated to a cytotoxic agent, such as, for example, auristatin E (dolastatin-10) or a derivative thereof.

In some embodiments, the tumor cell is a melanoma cell or a glioblastoma cell. In some embodiments, the melanoma cell comprise the NRAS or BRAF mutation.

In some embodiments, the ERK pathway inhibitor is selected from A6355 (3-(2-Aminoethyl)-5-((4-ethoxyphenyl)methylene)-2,4-thiazolidinedione hydrochloride) and FR180204 ((5-(2-phenylpyrazolo[1,5-a]pyridin-3-yl)-1H-pyrazolo[3,4-c]pyridazin-3-amine)). In some embodiments, the tyrosine kinase inhibitor is imatinib. In some embodiments, lysosomotropic weak base is ammonium chloride or chloroquine. In some embodiments, the inhibitor of GPNMB shedding is monensin. In some embodiments, the p38 MAK inhibitor is SB202190 (4-[4-(4-Fluorophenyl)-5-(4-pyridinyl)-1H-imidazol-2-yl]phenol) or SB203580 (4-[5-(4-Fluorophenyl)-2-[4-(methylsulfonyl)phenyl]-1H-imidazol-4-yl]pyridine).

The invention further provides methods for enhancing expression of GPNMB on the surface of a tumor cell, comprising contacting a tumor cell with a composition comprising an isolated monoclonal antibody that specifically binds to GPNMB and a second agent selected from an inhibitor of the ERK pathway, a tyrosine kinase inhibitor, an inhibitor of p38 MAPK, a lysosomotropic weak base and an inhibitor of GPNMB shedding, wherein the composition is present in an amount sufficient to increase expression of GPNMB on the tumor cell or decrease shedding of GPNMB by the tumor cell. In some embodiments, the antibody is a human monoclonal antibody.

In some embodiments, the antibody includes a heavy chain variable region comprising an amino acid sequence selected from the group consisting of SEQ ID NO: 2, 20, 38, 56, 74, 92, 110, 128, 146, 164, 182, 200, 218, 236, 253, 256, 260, 265, 270, 274, 277, 281 and 285; and the antibody includes a light chain variable region comprising an amino acid sequence selected from the group consisting of: SEQ ID NO: 11, 29, 47, 65, 83, 101, 119, 137, 155, 173, 191, 209, 227 and 245.

In some embodiments, the antibody includes (a) a V_{H} CDR1 region comprising the amino acid sequence of SEQ ID NO: 4, 22, 40, 58, 76, 94, 112, 130, 148, 166, 184, 202, 220, 238, 254, 257, 261, 266, 271, 278, 282 or 286; (b) a V_{H} CDR2 region comprising the amino acid sequence of SEQ ID NO: 6, 24, 42, 60, 78, 96, 114, 132, 150, 168, 186, 204, 222, 240, 255, 258, 262, 267, 272, 275, 279, 283 or 287; (c) a V_{H} CDR3 region comprising the amino acid sequence of SEQ ID NO: 8, 26, 44, 62, 80, 98, 116, 134, 152, 170, 188, 206, 224, 242, 259, 263, 264, 268, 269, 273, 276, 280, 284 or 288; (d) a V_{L} CDR1 region comprising the amino acid sequence of SEQ ID NO: 13, 31, 49, 67, 85, 103, 121, 139, 157, 175, 193, 211, 229 or 247; (e) a V_{L} CDR2 region comprising the amino acid sequence of SEQ ID NO: 15, 33, 51, 69, 87, 105, 123, 141, 159, 177, 195, 213, 231, 249 or 279; and (f) a V_{L} CDR3 region comprising the amino acid sequence of SEQ ID NO: 17, 35, 53, 71, 89, 107, 125, 143, 161, 179, 197, 215, 233 or 251. In some embodiments, the antibody is an IgG 1 antibody.

In some embodiments, the antibody is conjugated to a cytotoxic agent, such as, for example, auristatin E (dolastatin-10) or a derivative thereof.

In some embodiments, the tumor cell is a melanoma cell or a glioblastoma cell. In some embodiments, the melanoma cell comprise the NRAS or BRAF mutation.

In some embodiments, the ERK pathway inhibitor is selected from A6355 (3-(2-Aminoethyl)-5-((4-ethoxyphenyl)methylene)-2,4-thiazolidinedione hydrochloride) and FR180204 ((5-(2-phenylpyrazolo[1,5-a]pyridin-3-yl)-1H-pyrazolo[3,4-c]pyridazin-3-amine)). In some embodiments, the tyrosine kinase inhibitor is imatinib. In some embodiments, lysosomotropic weak base is ammonium chloride or chloroquine. In some embodiments, the inhibitor of GPNMB shedding is monensin. In some embodiments, the p38 MAK inhibitor is SB202190 (4-[4-(4-Fluorophenyl)-5-(4-pyridinyl)-1H-imidazol-2-yl]phenol) or SB203580 (4-[5-(4-Fluorophenyl)-2-[4-(methylsulfonyl)phenyl]-1H-imidazol-4-yl]pyridine).

The invention also provides methods of treating or preventing a disease associated with overexpression of GPNMB comprising administering to a subject in need thereof an effective amount of a composition comprising an isolated monoclonal antibody that specifically binds to GPNMB and a second agent selected from an inhibitor of the ERK pathway, a tyrosine kinase inhibitor, an inhibitor of p38 MAPK, a lysosomotropic weak base and an inhibitor of GPNMB shedding.

In some embodiments, the disease is melanoma or a neoplasm of CNS system. For example, the neoplasm of CNS system is astrocytoma, glioblastoma, medulloblastoma, or neoplastic meningitis. In some embodiments, the subject is human.

In some embodiments, the antibody is a human monoclonal antibody.

In some embodiments, the antibody includes a heavy chain variable region comprising an amino acid sequence selected from the group consisting of SEQ ID NO: 2, 20, 38, 56, 74, 92, 110, 128, 146, 164, 182, 200, 218, 236, 253, 256, 260, 265, 270, 274, 277, 281 and 285; and the antibody includes a light chain variable region comprising an amino acid sequence selected from the group consisting of: SEQ ID NO: 11, 29, 47, 65, 83, 101, 119, 137, 155, 173, 191, 209, 227 and 245.

In some embodiments, the antibody includes (a) a V_{H} CDR1 region comprising the amino acid sequence of SEQ ID NO: 4, 22, 40, 58, 76, 94, 112, 130, 148, 166, 184, 202, 220, 238, 254, 257, 261, 266, 271, 278, 282 or 286; (b) a V_{H} CDR2 region comprising the amino acid sequence of SEQ ID NO: 6, 24, 42, 60, 78, 96, 114, 132, 150, 168, 186, 204, 222, 240, 255, 258, 262, 267, 272, 275, 279, 283 or 287; (c) a V_{H} CDR3 region comprising the amino acid sequence of SEQ ID NO: 8, 26, 44, 62, 80, 98, 116, 134, 152, 170, 188, 206, 224, 242, 259, 263, 264, 268, 269, 273, 276, 280, 284 or 288; (d) a V_{L} CDR1 region comprising the amino acid sequence of SEQ ID NO: 13, 31, 49, 67, 85, 103, 121, 139, 157, 175, 193, 211, 229 or 247; (e) a V_{L} CDR2 region comprising the amino acid sequence of SEQ ID NO: 15, 33, 51, 69, 87, 105, 123, 141, 159, 177, 195, 213, 231, 249 or 279; and (f) a V_{L} CDR3 region comprising the amino acid sequence of SEQ ID NO: 17, 35, 53, 71, 89, 107, 125, 143, 161, 179, 197, 215, 233 or 251. In some embodiments, the antibody is an IgG1 antibody.

In some embodiments, the antibody is conjugated to a cytotoxic agent, such as, for example, auristatin E (dolastatin-10) or a derivative thereof.

In some embodiments, the tumor cell is a melanoma cell or a glioblastoma cell. In some embodiments, the melanoma cell comprise the NRAS or BRAF mutation.

In some embodiments, the ERK pathway inhibitor is selected from A6355 (3-(2-Aminoethyl)-5-((4-ethoxyphenyl)methylene)-2,4-thiazolidinedione hydrochloride) and FR180204 ((5-(2-phenylpyrazolo[1,5-a]pyridin-3-yl)-1H-pyrazolo[3,4-c]pyridazin-3-amine)). In some embodiments, the tyrosine kinase inhibitor is imatinib. In some embodiments, lysosomotropic weak base is ammonium chloride or chloroquine. In some embodiments, the inhibitor of GPNMB shedding is monensin. In some embodiments, the p38 MAK inhibitor is SB202190 (4-[4-(4-Fluorophenyl)-5-(4-pyridinyl)-1H-imidazol-2-yl]phenol) or SB203580 (4-[5-(4-Fluorophenyl)-2-[4-(methylsulfonyl)phenyl]-1H-imidazol-4-yl]pyridine).

Targeting GPNMB on tumor cells is useful to treat a subject at risk for or afflicted with cancer. Such a subject would benefit from treatment with an anti-GPNMB antibody of the present invention. Typically, antibodies are administered in an outpatient setting by weekly administration at about 0.1-1.0 mg/kg dose by slow intravenous (IV) infusion. The appropriate therapeutically effective dose of an antibody is selected by a treating clinician and would range approximately from 1 μg/kg to 20 mg/kg, from 1 μg/kg to 10 mg/kg, from 1 μg/kg to 1 mg/kg, from 10 μg/kg to 1 mg/kg, from 10 μg/kg to 100 μg/kg, from 100 μg/kg to 1 mg/kg, and from 500 μg/kg to 5 mg/kg.

Additional aspects of the disclosure will be set forth in part in the description which follows, and in part will be obvious from the description, or may be learned by practicing the invention.

### BRIEF DESCRIPTION OF THE FIGURES

FIGS. 1A-1B are a series of illustrations depicting the expression of GPNMB and other melanoma-associated targets in cell lines representative of various cancer types. (A) Microarray transcript analysis of melanoma-associated targets on the NCI-60 panel of cancer cell lines. The cell lines marked with an asterisk (MDA-MB-435 and MDA-N) were originally classified as breast carcinomas, but are actually melanomas; note that MDA-N is a subline of MDA-MB-435). (B) Immunoblotting of melanoma-associated targets on whole-cell lysates harvested from the indicated cancer cell lines. Information on the cell lines categorized as “miscellaneous” is as follows: NCIH23 (lung); MDAMB231 (breast); OVCAR5 (ovarian); CAKI2 (renal); HT29 (colon). Additional nomenclature on the targets is as follows: GPNMB (Glycoprotein NMB); MARTI (Melanoma Antigen Recognized by T Cells 1; MLANA; MELAN A); TYRP2 (Tyrosine-Related Protein 2; TRP2; DCT; Dopachrome Tautomerase); TYRP1 (Tyrosine-Related Protein 1; TRP1; GP75); PMEL17 (Melanocyte Protein 17; SILV; GP100); MAGEA1 (Melanoma Antigen Family A1); MCSP (CSPG4; MEL-CSPG; Melanoma-Associated Chondroitin Sulfate Proteoglycan 4); MTf (MFI2; Melanotransferrin; Melanoma-Associated Antigen P97); MCAM (Melanoma Adhesion Molecule; CD146; MUC18). See Table 36 for additional information on the cell lines. An antibody to ERK was used as a loading control.

FIGS. 2A-2B are a series of illustrations depicting the effect of various drugs on the expression of GPNMB in melanoma cells. (A) Immunoblotting for GPNMB on whole-cell lysates harvested from A375 melanoma cells following exposure to the indicated compounds for 24 hours. Drug concentrations were as follows: RAFKi: 553013 (10 μM); MEKi: U0126 (20 μM); MEKi: PD98059 (40 μM); MEKi: 444939 (50 μM); ERKi: A6355 (50 μM); RAFKi: GW5074 (10 μM); ERKi: FR180204 (50 μM); p38 MAPKi: SB202190 (10 μM); p38 MAPKi: SB203580 (50 μM); aurora kinase inhibitor: (100 μM); INK inhibitor: (100 μM); HSP90i: geldanamycin (1 μM). An antibody to phospho-ERK (pp-ERK) was used to determine the level of ERK activation, and an antibody to actin was used as a loading control. (B) Flow cytometry analysis was performed to quantitate the amount of GPNMB surface expression on intact, non-permeabilized, A375 cells following exposure to the indicated MEK inhibitors for 48 hours.

FIGS. 3A-3B are a series of illustrations depicting the effect of ERK-pathway inhibitors on the expression of GPNMB in cell lines representative of various cancer types. (A) Immunoblotting for GPNMB on whole-cell lysates harvested from various cancer cell lines following exposure to the indicated compounds for 48 hours. Compounds and concentrations were as follows: MEKi: U0126 (20 μM); RAFKi: 553013 (10 μM); ERKi: FR180204 (50 μM). The top panel consists of melanoma cell lines harboring mutations in NRAS (SKMEL2) or BRAF (A375, WM2664, G361, SKMEL28, UACC62). The bottom panel consists of a melanoma (MEWO) and a glioblastoma (SF539) cell line known to be wild-type for NRAS and BRAF, two glioblastoma cell lines (U118MG, XF498) for which the NRAS/BRAF mutational status is unknown, a colon cancer cell line (HT29) which harbors a BRAF mutation and a fibrosarcoma cell line (HT1080) which harbors an NRAS mutation. (B) Immunoblotting for GPNMB on whole-cell lysates harvested from various cancer cell lines following exposure to the indicated compounds for 12, 24 or 48 hours. Compounds were as follows: MEKi (top panel): U0126; RAFKi (middle panel): 553013; ERKi (bottom panel): FR180204. Drug concentrations shown are in μM. The cell lines utilized (A375, SKMEL2, SF539) are described above. See Table 36 for additional details on the cell lines used in this figure. In both (A) and (B), an antibody to phospho-ERK was used to determine the level of ERK activation and an antibody to ERK was used as a loading control.

FIGS. 4A-4C are a series of illustrations depicting the effect of MEKi pretreatment on the growth-inhibitory activity of CR011-vcMMAE towards melanoma cells. UACC62 melanoma cells, which harbor a BRAF mutation and exhibit robust GPNMB induction in response to inhibitors of the ERK pathway, were incubated in the absence or presence of a MEKi (U0126; 1 μM) or for 48 hours. After this time, cells were washed and replated in the absence or presence of CR011-vcMMAE at the indicated concentrations for 72 hours. Pictures (A) and cell counts (B) were then taken from cells that had been incubated in the absence of both the MEK1 and CR011-vcMMAE (Control), in the presence of either the MEK1 or CR011-vcMMAE, or in the presence of the MEKi followed by CR011-vcMMAE. The concentration of CR011-vcMMAE used in (A) was 0.16 μg/mL. In (B), viable cells were counted by trypan blue dye exclusion and cell numbers were plotted relative to untreated control cells.

FIG. 5 is an illustration depicting the effect of ERK pathway inhibition on the expression of various melanoma-associated targets in melanoma cell lines harboring mutations in NRAS or BRAF. Immunoblotting for GPNMB and other melanoma-associated targets on whole-cell lysates harvested from the indicated cell lines following exposure to a MEKi (U0126; 20 μM) for 48 hours. See Table 36 for details on the cell lines. An antibody to phospho-ERK was used to determine the level of ERK activation and an antibody to ERK was used as a loading control.

FIGS. 6A-6E are a series of illustrations depicting Identification of drugs that increase the expression of GPNMB without inhibiting the ERK pathway. (A) Immunoblotting for GPNMB on whole-cell lysates harvested from various melanoma cell lines following exposure to the indicated compounds for 48 hours. Compounds and concentrations were as follows: MEKi: U0126 (10 μM); imatinib (20 μM); p38i (p38 MAPKi SB202190; 50 μM); NH₄C1 (ammonium chloride; 20 mM); CLQ (chloroquine; 20 μM). (B) Immunoblotting for GPNMB on whole-cell lysates harvested from various cell lines following exposure to the MEKi (U0126; 10 μM) or imatinib (20 μM) for 48 hours. (C) Immunoblotting for GPNMB on whole-cell lysates harvested from various cell lines following exposure to the MEKi (U0126; 10 μM) or p38i (p38 MAPKi SB230580; 50 μM) for 48 hours. (D) Immunoblotting for GPNMB on whole-cell lysates harvested from various cell lines following exposure to the protein synthesis inhibitors emetine (5 μg/mL) or cyclohexamide (20 μg/mL) in the presence or absence of NH₄Cl (50 mM) for 1 hour. (E) Immunoblotting for GPNMB and other melanoma-associated targets on whole-cell lysates harvested from various cell lines following exposure to emetine (5 μg/mL) or NH₄Cl (20 mM) for 48 hours. Information on the cell lines used in this figure is as follows: A375 and WM2664 are melanomas harboring mutations in BRAF, SKMEL2 is a melanoma harboring an NRAS mutation, MEWO is a melanoma wild-type for NRAS and BRAF, SF539 is a glioblastoma wild-type for NRAS and BRAF, and XF498 is a glioblastoma for which the mutational status of NRAS/BRAF is unknown (see Table 36). An antibody to ERK was used as a loading control and in some cases an antibody to phospho-ERK was used to determine the level of ERK activation.

FIG. 7A-7B are a series of illustrations depicting the Identification of drugs that inhibit GPNMB shedding by tumor cells. (A) Immunoblotting for GPNMB on whole-cell lysates (P) and supernatants (S) harvested from WM2664 and UACC62 melanoma cell lines following exposure to the indicated compounds for 48 hours. Compounds and concentrations were as follows: MEKi: U0126 (10 μM); imatinib (20 μM); NH₄Cl (ammonium chloride; 20 mM); CLQ (chloroquine; 20 μM); MON (monensin; 330 nM); MMPi (metalloprotease inhibitor GM6001; 25 μM). (B) Immunoblotting for GPNMB on whole-cell lysates (P) and supernatants (S) harvested from the WM2664 melanoma cell line following exposure to the indicated compounds/concentrations for 48 hours. The vehicle for MON (monesin) was ethanol and the vehicle for the MMPi (GM6001) was DMSO. Compounds and concentrations were as follows: MEKi: U0126 (10 μM); imatinib (20 μM); NH₄Cl (ammonium chloride; 20 mM); CLQ (chloroquine; 20 μM); MON (monensin; 330 nM); MMPi (metalloprotease inhibitor GM6001; 25 μM). An antibody to ERK was used as a loading control for whole-cell lysates.

FIG. 8: The chemical structure of Maleimidocoaproyl-Valine-Citrullin-Monomethyl-Auristatin E (vcMMAE).

FIG. 9: Disulfides on CR011 antibody are gently reduced in the presence of TCEP to generate ˜4 thiols per Ab. vcMMAE is then added to antibody solution. Nucleophilic attack of thiolates on maleimide-groups results in a stable thioester linkage. The resulting conjugate is purified from the mixture.

### DETAILED DESCRIPTION OF THE INVENTION

GPNMB is a type IA cell-surface glycoprotein expressed by certain types of cancers including malignant melanoma and glioblastoma. (Tse, K. F., et al., CR011, a fully human monoclonal antibody-auristatin E conjugate, for the treatment of melanoma. Clin Cancer Res, 12: 1373-1382, 2006; and Kuan, C. T., et al., Glycoprotein nonmetastatic melanoma protein B, a potential molecular therapeutic target in patients with glioblastoma multiforme. Clin Cancer Res, 12: 1970-1982, 2006). Antibodies to the extracellular domain of GPNMB have been previously developed, e.g., in WO06/071441, the contents of which are hereby incorporated by reference in their entirety. These ant-GPNMB antibodies were also conjugated to the cytotoxic drug monomethylauristatin E (MMAE) via a protease-sensitive linker to facilitate release of drug following internalization. Studies have found that this antibody-drug conjugate (ADC) effectively targeted GPNMB-expressing tumor cells in vitro and in xenograft models. (Tse, K. F., et al., CR011, a fully human monoclonal antibody-auristatin E conjugate, for the treatment of melanoma. Clin Cancer Res, 12: 1373-1382, 2006; and Pollack, V. A., et al., Treatment parameters modulating regression of human melanoma xenografts by an antibody-drug conjugate (CR011-vcMMAE) targeting GPNMB. Cancer Chemother Pharmacol, 60: 423-435, 2007).

Toward the goal of maximizing the anticancer activity of anti-GPNMB antibodies and immunoconjugates such as the CR011-vcMMAE immunoconjugate, a variety of compounds were screened for the ability to increase expression of GPNMB on the surface of tumor cells and/or decrease shedding of GPNMB by tumor cells. The screens led to the identification of compounds falling into two main categories. The first of these categories is comprised of inhibitors of the ERK pathway which strongly increased GPNMB expression in melanoma cells harboring activating mutations in NRAS or BRAF, which account for ˜80% of all melanomas. The ERK pathway represents a clinically relevant pathway in oncology drug development, and pharmacological inhibitors of this pathway appear to increase GPNMB expression via a transcriptional mechanism. An examination of a variety of melanoma-associated targets other than GPNMB demonstrated that some, but not all of these other proteins, were also induced by inhibitors of the ERK pathway in melanoma cells and that by way of comparison, GPNMB was one of the most consistently expressed and strongly induced proteins among those surveyed. Melanoma cells exposed to inhibitors of the ERK pathway were sensitized to the growth-inhibitory effects of CR011-vcMMAE. The second category of compounds that increased GPNMB surface expression did so in both melanoma and glioblastoma cells regardless of NRAS/BRAF mutational status, apparently by enhancing the stability and/or membrane localization of GPNMB. A third group of compounds simultaneously inhibited GPNMB shedding and increased the surface expression of GPNMB on tumor cells. The compounds, compositions and methods provided herein maximize the activity of anti-GPNMB antibodies and/or immunoconjugates such as the CR011-vcMMAE, through use in combination with compounds that enhance the cell-surface expression of GPNMB and/or decrease GPNMB shedding.

A variety of parameters can potentially influence the activity of a given ADC (Carter, P. Improving the efficacy of antibody-based cancer therapies. Nat Rev Cancer, 1: 118-129, 2001). For example, cancers often exhibit intra/inter-tumoral heterogeneity of target expression which may affect ADC activity since a minimum threshold density of target molecules at the cell-surface may be required for efficient tumor-targeting. The rates of internalization and processing of the target-ADC complex, steps which are required for the intracellular delivery of active cytotoxic drug within tumor cells, are also important. Another parameter that can influence ADC activity is that of target shedding. Shedding of plasma membrane proteins is a common phenomena which can potentially reduce ADC activity by decreasing the expression of the relevant target on the surface of tumor cells and by generating soluble target protein that may compete with surface-associated protein for ADC binding (Eichenauer, D. A., et al., ADAM10 inhibition of human CD30 shedding increases specificity of targeted immunotherapy in vitro. Cancer Res, 67: 332-338, 2007; and Dello Sbarba, P. and Rovida, E. Transmodulation of cell surface regulatory molecules via ectodomain shedding. Biol Chem, 383: 69-83, 2002).

The studies provided herein focused on the two cancer types that are associated with GPNMB expression: melanomas and glioblastomas. Melanomas can be categorized based on mutational status, with approximately 15-30% and 50-70% of these tumors harboring activating mutations in either NRAS or BRAF, respectively, and a small percentage possessing wild-type NRAS/BRAF (Gray-Schopfer, V., et al., Melanoma biology and new targeted therapy. Nature, 445: 851-857, 2007). Cell lines representing each of these genetically distinct melanoma subtypes were included in the present investigation. In contrast to melanomas, glioblastomas rarely harbor activating NRAS or BRAF mutations (Knobbe, C. B., et al., Mutation analysis of the Ras pathway genes NRAS, HRAS, KRAS and BRAF in glioblastomas. Acta Neuropathol (Berl), 108: 467-470, 2004).

Tumor cells harboring mutations NRAS or BRAF mutations exhibit constitutive activation of the ERK signaling pathway which is comprised of the RAS GTPase and three kinases: RAF, MEK and ERK (Dhillon, A. S., et al., MAP kinase signaling pathways in cancer. Oncogene, 26: 3279-3290, 2007). In normal quiescent cells, the components of this pathway are enzymatically inactive. Following an appropriate stimulus such as a growth-factor receptor-ligand interaction, the components of the ERK. pathway are sequentially activated, culminating in the regulation of numerous cytoplasmic and nuclear proteins via either direct or indirect ERK-mediated phosphorylation. In contrast to the situation that exists in normal cells, tumors harboring mutations in NRAS or BRAF exhibit constitutive activation of the ERK pathway in the absence of external stimuli.

In addition to identifying compounds that increased the expression of GPNMB, experiments were performed to determine whether such compounds could be used to enhance the anticancer activity of CR011-vcMMAE, as shown in the Examples provided herein. Finally, both basal and inducible GPNMB expression was compared with that of other melanoma-associated tumor targets to view these results regarding GPNMB in a broader context. The results presented herein extend the understanding of GPNMB as a cancer target, illustrate some unique attributes of this molecule, and demonstrate that the activity of anti-GPNMB antibodies and/or immunoconjugates such as CR011-vcMMAE is maximized by use in combination with compounds that increase the surface expression and/or decrease the shedding of GPNMB.

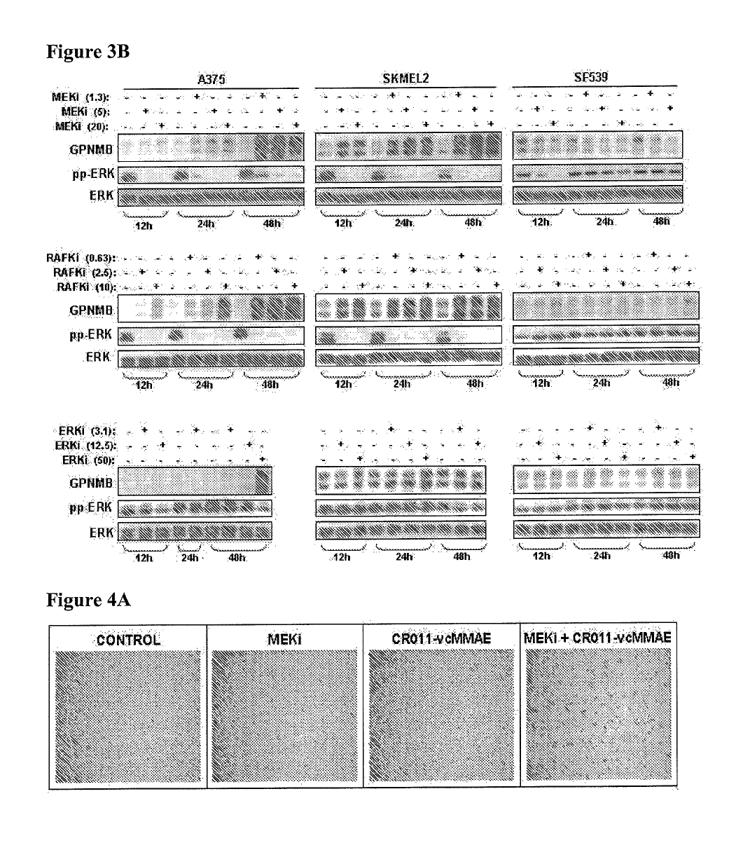

As shown in the Examples provided herein, inhibitors of the ERK pathway induced GPNMB expression in melanoma cell lines harboring NRAS or BRAF mutations, but not in a melanoma cell line which possessed wild-type NRAS/BRAF or in non-melanoma cancer cell lines possessing wild-type or mutant NRAS/BRAF. GPNMB is often subjected to partial transcriptional repression in melanoma cells possessing a constitutively activated ERK pathway, and inhibitors of the ERK pathway relieve this repression. The timecourse data (FIG. 3B) is consistent with ERK inhibitors inducing GPNMB expression at the transcriptional level, as is microarray data generated by Shields et. al. in which GPNMB transcript levels were found to be significantly increased in BRAF-mutant melanoma cell lines following exposure to a MEK inhibitor (Shields, J. M., et al., Lack of extracellular signal-regulated kinase mitogen-activated protein kinase signaling shows a new type of melanoma. Cancer Res, 67: 1502-1512, 2007). The observed lack of GPNMB induction by ERK pathway inhibitors in non-melanoma cell lines harboring mutations in BRAF (HT29 colon carcinoma) or NRAS (HT1080 fibrosarcoma) indicates that such cell types lack crucial transcription factors required for GPNMB expression. It should be noted that although GPNMB may be transcriptionally repressed as a consequence of constitutive ERK activation, the fact that at least some expression of GPNMB transcript and protein is detected in most melanoma cell lines harboring NRAS or BRAF mutations indicates that this transcriptional repression is generally incomplete.

There are a number of reports demonstrating the induction of genes other than GPNMB in response to inhibitors of the ERK pathway. For example, the aforementioned microarray data generated by Shields et. al. lists a number of genes that are transcriptionally increased in BRAF-mutant melanoma cells following exposure to a MEK inhibitor (Shields, J. M., et al., Lack of extracellular signal-regulated kinase mitogen-activated protein kinase signaling shows a new type of melanoma. Cancer Res, 67: 1502-1512, 2007). One of these genes, TYRP-2, encodes a protein involved in normal melanogenesis, and the results show that TYPR-2 is induced at the protein level following exposure of melanoma cell lines to a MEK inhibitor. Others have shown that melanoma cell lines exhibit evidence of phenotypic differentiation in response to inhibition of the ERK pathway, concomitant with increased expression of proteins which play a role in normal melanogenesis (Englaro, W., et al., Inhibition of the mitogen-activated protein kinase pathway triggers B 16 melanoma cell differentiation. J Biol Chem, 273: 9966-9970, 1998; Kono, M., et al., Role of the mitogen-activated protein kinase signaling pathway in the regulation of human melanocytic antigen expression. Mol Cancer Res, 4: 779-792, 2006; and Koo, H. M., et al., Apoptosis and melanogenesis in human melanoma cells induced by anthrax lethal factor inactivation of mitogen-activated protein kinase kinase. Proc Natl Acad Sci USA, 99: 3052-3057, 2002), and that melanoma progression is associated with reduced expression of differentiation-associated genes (Ryu, B., et al., Comprehensive expression profiling of tumor cell lines identifies molecular signatures of melanoma progression. PLoS ONE, 2: e594, 2007). Thus, a constitutively activated ERK pathway, which is present in most melanomas as a consequence of NRAS or BRAF mutation, actively participates in the maintenance of a dedifferentiated state in melanoma cells by suppressing the expression of differentiation-associated genes. A similar scenario has recently been described for thyroid cancer where a constitutively activated ERK pathway in tumor cells was associated with the loss of expression of iodide-metabolizing genes, and whose expression was restored, with therapeutic implications, following inhibition of the ERK pathway (Liu, D., et al., Suppression of BRAF/MEK/MAP kinase pathway restores expression of iodide-metabolizing genes in thyroid cells expressing the V600E BRAF mutant. Clin Cancer Res, 13: 1341-1349, 2007).

While the precise function of GPNMB in normal melanocytes is unknown, evidence suggests that this protein is required for normal melanosome function (Anderson, M. G., et al., Mutations in genes encoding melanosomal proteins cause pigmentary glaucoma in DBA/2J mice. Nat Genet, 30: 81-85, 2002). Thus, the finding that inhibitors of the ERK pathway specifically induce GPNMB expression in melanomas harboring mutations in NRAS or BRAF may represent another example of a differentiation-associated gene that is repressed in cancers possessing a constitutively activated ERK pathway, and inducible in response to the pharmacological inhibition of this signaling pathway. The findings provided herein are used for the treatment of malignant melanomas harboring NRAS or BRAF mutations by using inhibitors of the ERK pathway (to induce GPNMB expression) in combination with an anti-GPNMB antibody and/or immunoconjugate such as the GPNMB-targeting ADC, CR011-vcMMAE. There is already an FDA-approved inhibitor of the ERK pathway (sorafenib, which inhibits RAF) and a number of others are in clinical development (Gray-Schopfer, V., et al., Melanoma biology and new targeted therapy. Nature, 445: 851-857, 2007; and Roberts, P. J. and Der, C. J. Targeting the Raf-MEK-ERK mitogen-activated protein kinase cascade for the treatment of cancer. Oncogene, 26: 3291-3310, 2007). It is interesting to note that inhibitors of the ERK pathway have previously been shown to sensitize tumor cells to antibody-based therapy (anti-EGFR) in preclinical studies (Benvenuti, S., et al., Oncogenic activation of the RAS/RAF signaling pathway impairs the response of metastatic colorectal cancers to anti-epidermal growth factor receptor antibody therapies. Cancer Res, 67: 2643-2648, 2007), and that the combination of an ERK pathway inhibitor (sorafenib) with an anti-EGFR antibody (cetuximab) is currently being examined in a clinical trial.

In addition to inhibitors of the ERK pathway, it was also found that GPNMB was strongly induced by imatinib, an FDA-approved inhibitor of multiple tyrosine kinases (c-Kit, PDGFR, Bcr-Abl). However, in contrast to what was found with inhibitors of the ERK pathway, imatinib induced GPNMB expression in both melanoma and glioblastoma cell lines regardless of NRAS/BRAF mutational status, and did so without inhibiting the ERK pathway. Thus, the combination of imatinib and CR011-vcMMAE is potentially useful for the treatment of both melanoma and glioblastoma. The intracellular domain of GPNMB possesses a sequence (YNPI), which matches the consensus for a tyrosine-based sorting motif that has been associated with rapid internalization of membrane proteins and whose activity is regulated via phosphorylation of the tyrosine present in the motif (Bonifacino, J. S, and Traub, L. M. Signals for sorting of transmembrane proteins to endosomes and lysosomes. Annu Rev Biochem, 72: 395-447, 2003). Imatinib increases GPNMB expression through increased protein stability due to altered sorting and/or processing as a consequence of changes in the phosphorylation status of GPNMB. Imatinib is currently being examined in a clinical trial for the treatment of melanoma in patients whose tumors harbor c-KIT mutations.

Like imatinib, inhibitors of p38 MAPK increased the expression of GPNMB in both melanoma and glioblastoma cell lines regardless of NRAS/BRAF mutational status and did so without inhibiting the ERK-pathway in cells possessing wild-type NRAS/BRAF. However, in contrast to imatinib, p38 MAPK inhibitors did inhibit the ERK pathway in melanoma cells harboring BRAF mutations. Thus, while an inhibition of the ERK pathway provides a mechanism by which p38 MAPK inhibitors induce GPNMB expression in melanomas possessing mutant BRAF, this cannot be the sole mechanism by which these compounds increase the expression of GPNMB. p38 MAPK has been shown to influence the downregulation and trafficking of the EGFR and p38 MAPK inhibitors enhanced the stability of this receptor (Frey, M. R., et al., p38 kinase regulates epidermal growth factor receptor downregulation and cellular migration. Embo J, 25: 5683-5692, 2006). Thus, p38 MAPK inhibition similarly increases GPNMB expression through enhanced protein stability. There are currently a number of p38 MAPK inhibitors under clinical investigation (Roberts, P. J. and Der, C. J. Targeting the Raf-MEK-ERK mitogen-activated protein kinase cascade for the treatment of cancer. Oncogene, 26: 3291-3310, 2007).

Other compounds which induced GPNMB expression in the present investigation were ammonium chloride and chloroquine, which are both lysosomotropic weak bases. Since these compounds neutralize the acidic environment of endocytic vesicles thereby causing decreased activity of resident acidic proteases, GPNMB expression is induced by these compounds as a consequence of reduced GPNMB degradation. The finding that GPNMB protein has a short half-life which is extended in the presence of ammonium chloride is consistent with this hypothesis. Since the activity of CR011-vcMMAE is dependent upon functional endosome/lysosome-mediated proteolytic activity to facilitate dissociation of antibody/toxin moieties following ADC internalization, the benefit of this drug combination is best realized by sequentially treating cancer cells first with a lysosomotropic compound (to induce GPNMB expression) followed by CR011-vcMMAE, rather than using these drugs simultaneously.

Compounds that inhibit GPNMB shedding were also identified in the present study. Many plasma membrane proteins undergo shedding whereby the extracellular domain of the protein is proteolytically cleaved at a membrane-proximal location thereby liberating a portion of the protein (called the ectodomain) into the extracellular environment (Dello Sbarba, P. and Rovida, E. Transmodulation of cell surface regulatory molecules via ectodomain shedding. Biol Chem, 383: 69-83, 2002). Proteases which perform the proteolytic cleavage of transmembrane proteins are collectively called “sheddases”, and members of the ADAMs family of membrane-anchored metalloproteases, particularly ADAM10 and 17, possess sheddase activity (Seals, D. F. and Courtneidge, S. A. The ADAMs family of metalloproteases: multidomain proteins with multiple functions. Genes Dev, 17: 7-30, 2003). Sheddase inhibitors have been developed for clinical indications (Zhou, B. B., et al., Targeting ADAM-mediated ligand cleavage to inhibit HER3 and EGFR pathways in non-small cell lung cancer. Cancer Cell, 10: 39-50, 2006; and Fridman, J. S., et al., Selective inhibition of ADAM metalloproteases as a novel approach for modulating ErbB pathways in cancer. Clin Cancer Res, 13: 1892-1902, 2007) and at least one (INCB7839) is currently in clinical trials. The strong inhibition of GPNMB shedding by the carboxylic ionophore monensin was unexpected since there are very few reports in the literature investigating the effects of this compound on ectodomain shedding. Monensin was shown to potentiate the antitumor activity of immunotoxins in xenograft models via an unknown mechanism (Griffin, T., et al., Potentiation of antitumor immunotoxins by liposomal monensin. J Natl Cancer Inst, 85: 292-298, 1993). In cells in which GPNMB expressed, GPNMB generally appears as two predominant species of ˜130 and 110 kDa when immunoblotted from whole-cell lysates. However, since only a single protein of ˜120 kDa is present in shed form, only the 130 kDa cell-associated GPNMB protein is subjected to shedding. The fact that monensin strongly reduces the level of the 130 kDa cell-associated GPNMB protein and increases the level of the 110 kDa cell-associated GPNMB protein explains the inhibition of GPNMB shedding by monensin. It has been determined by immunoprecipitation that the CR011 anti-GPNMB monoclonal antibody does in fact recognize shed GPNMB. Shed GPNMB competes with tumor cell-associated GPNMB for reactivity with CR011-vcMMAE, which reduces the anticancer activity of this ADC. Thus, the inhibitors of GPNMB shedding identified herein enhance the activity of CR011-vcMMAE.

A straightforward mechanistic rationale for using the CR011-vcMMAE in combination with compounds that increase the surface expression of GPNMB on tumor cells is that this would allow for more efficient tumor-targeting of CR011-vcMMAE, which would in turn translate into enhanced anti-tumor activity by this ADC. However, additional mechanisms independent of effects on GPNMB expression are also envisioned as contributing to a favorable outcome upon using CR011-vcMMAE in combination with these other drugs. For example, metastatic malignant melanoma does not respond well to single-agent chemotherapy and available evidence suggests that there may be a therapeutic benefit from simultaneously targeting multiple signaling pathways in this malignancy (Smalley, K. S., et al., Multiple signaling pathways must be targeted to overcome drug resistance in cell lines derived from melanoma metastases. Mol Cancer Ther, 5: 1136-1144, 2006). Thus, the combined use of CR011-vcMMAE (whose auristatin-based cytotoxic moiety targets tubulin) together with a compound that targets a different pathway (such as an inhibitor of the ERK pathway) is therapeutically beneficial due to a simultaneous attack on two different pathways. Auristatin has previously been shown to synergize with other anticancer agents to induce tumor growth inhibition (Mohammad, R. M., et al., Successful treatment of human chronic lymphocytic leukemia xenografts with combination biological agents auristatin PE and bryostatin 1. Clin Cancer Res, 4: 1337-1343, 1998). Also, since inhibition of the ERK pathway has been shown to decrease the expression of P-glycoprotein (Katayama, K., Yoshioka, S., Tsukahara, S., Mitsuhashi, J., and Sugimoto, Y. Inhibition of the mitogen-activated protein kinase pathway results in the down-regulation of P-glycoprotein. Mol Cancer Ther, 6: 2092-2102, 2007), using CR011-vcMMAE in combination with an inhibitor of the ERK pathway enhances the anticancer activity of CR011-vcMMAE by increasing the intracellular retention of the MMAE drug moiety within tumor cells. Finally, the relatively long half-life of antibody-based drugs such as CR011-vcMMAE is particularly advantageous for use in combination due to dosing/scheduling considerations.

In summary, a number of compounds that increase the expression and/or decrease the shedding of GPNMB have been identified, and the results provided in the Examples herein have shown that such compounds are useful in enhancing the growth-inhibitory activity of anti-GPNMB antibodies and/or immunoconjugates such as CR011-vcMMAE towards cancer cell lines. These studies support the evaluation of CR011-vcMMAE used in combination with other drugs for the treatment of metastatic malignant melanoma and glioblastoma.

As used herein, the term “antibody” refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, engineered, and grafted antibodies. Unless otherwise modified by the term “intact,” as in “intact antibodies,” for the purposes of this disclosure, the term “antibody” also includes antibody fragments such as Fab, F(ab′)₂, Fv, scFv, bi-scFv, bi-Ab, Fd, dAb, and other antibody fragments that retain antigen-binding function, i.e., the ability to bind GPNMB specifically. Typically, such fragments would comprise an antigen-binding domain.

As used herein, the terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.”

An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

As used herein, the term “repertoire” refers to a genetically diverse collection of nucleotides derived wholly or partially from sequences that encode expressed immunoglobulins. The sequences are generated by in vivo rearrangement of, e.g., V, D, and J segments for H chains and, e.g., V and J segment for L chains. Alternatively, the sequences may be generated from a cell line by in vitro stimulation, in response to which the rearrangement occurs. Alternatively, part or all of the sequences may be obtained by combining, e.g., unrearranged V segments with D and J segments, by nucleotide synthesis, randomised mutagenesis, and other methods, e.g., as disclosed in U.S. Pat. No. 5,565,332.

As used herein, the terms “specific interaction” and “specific binding” refer to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant K_{A} is higher than 10⁶ M⁻¹, or more preferably higher than 10⁸ M⁻¹. If necessary, non-specific binding can be reduced without substantially affecting specific binding by varying the binding conditions. The appropriate binding conditions such as concentration of antibodies, ionic strength of the solution, temperature, time allowed for binding, concentration of a blocking agent (e.g., serum albumin, milk casein), etc., may be optimized by a skilled artisan using routine techniques.

As used herein, the term “substantially as set out” refers that the relevant CDR, V_{H}, or V_{L} domain of the invention will be either identical to or have only insubstantial differences in the specified regions (e.g., a CDR), the sequence of which is set out. Insubstantial differences include minor amino acid changes, such as substitutions of 1 or 2 out of any 5 amino acids in the sequence of a specified region.

As used herein, the term “CR011” refers to the fully human monoclonal antibody that specifically binds to GPNMB referred to as Mab 1.15.1 in the instant invention.

The terms “GPNMB” and “CG56972” are used interchangeably herein. As used herein, the terms “GPNMB” or “CG56972” refer to a transmembrane glycoprotein that has an amino acid sequence as set forth in SEQ ID NO: 289, an analog, derivative or a fragment thereof, or a fusion protein comprising GPNMB, an analog, derivative or a fragment thereof. In certain embodiments, the term “GPNMB” refers to the mature, processed form of GPNMB. In other embodiments, the term “GPNMB” refers to the extracellular domain of GPNMB.

As used herein, the term “GPNMB activity” refers to one or more activities associated with GPNMB. To “modulate” GPNMB activity is to alter the baseline results observed with, and that can be attributed to GPNMB. To “neutralize” GPNMB is to cancel one or more effects, e.g. activity observed with, and that can be attributed to GPNMB.

As used herein, the term “isolated” refers to a molecule that is substantially free of its natural environment. For instance, an isolated protein is substantially free of cellular material or other proteins from the cell or tissue source from which it is derived. The term “isolated” also refers to preparations where the isolated protein is sufficiently pure to be administered as a pharmaceutical composition, or at least 70-80% (w/w) pure, more preferably, at least 80-90% (w/w) pure, even more preferably, 90-95% pure; and, most preferably, at least 95%, 96%, 97%, 98%, 99%, or 100% (w/w) pure.

As used herein, the term “inhibit” or “inhibition of” refers to reducing by a measurable amount, or to prevent entirely.

As used herein, the term “Cytotoxic effect” in reference to the effect of an agent on a cell, means killing of the cell. “Cytostatic effect” refers to an inhibition of cell proliferation. A “cytotoxic agent” refers an agent that has a cytotoxic or cytostatic effect on a cell, thereby depleting or inhibiting the growth of, respectively, cells within a cell population.

As used herein, the terms “prevent,” “preventing,” and “prevention” refer to the inhibition of the development or onset of a disorder associated with aberrant expression and/or activity of GPNMB (e.g., cancer) or the prevention of the recurrence, onset, or development of one or more symptoms of a disorder associated with aberrant expression and/or activity of GPNMB (e.g., cancer) in a subject resulting from the administration of a therapy or the administration of a combination of therapies.

As used herein, the term “effective amount” refers to a dosage or amount that is sufficient to reduce the activity of GPNMB to result in amelioration of symptoms in a patient or to achieve a desired biological outcome.

As used herein, the term “prophylactically effective amount” refers to the amount of a therapy which is sufficient to result in the prevention of the development, recurrence, or onset of a disorder associated with aberrant expression and/or activity of GPNMB (e.g., cancer) or one or more symptoms thereof, or to enhance or improve the prophylactic effect(s) of another therapy.

As used herein, a “protocol” includes dosing schedules and dosing regimens. The protocols herein are methods of use and include prophylactic and therapeutic protocols.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey, such as a cynomolgous monkey, chimpanzee, and a human), and more preferably a human.

As used herein, the terms “therapeutic agent” and “therapeutic agents” refer to an agent that can be used in the prevention, treatment, management, or amelioration of a disorder associated with aberrant expression and/or activity of GPNMB (e.g., cancer) or one or more symptoms thereof. In certain embodiments, the term “therapeutic agent” refers to an antibody that immunospecifically binds to GPNMB. In certain other embodiments, the term “therapeutic agent” refers an agent other than an antibody that immunospecifically binds to GPNMB.

As used herein, the terms “therapies” and “therapy” can refer to any protocol(s), method(s), and/or agent(s) that can be used in the prevention, treatment, management, or amelioration of a disorder associated with aberrant expression and/or activity of GPNMB (e.g., cancer) or one or more symptoms thereof. In certain embodiments, the terms “therapies” and “therapy” refer to anti-cancer therapy, biological therapy, supportive therapy, and/or other therapies useful in treatment, management, prevention, or amelioration of cancer or one or more symptoms thereof known to one of skill in the art such as medical personnel.

As used herein, the terms “treat,” “treatment,” and “treating” refer to the eradication, removal, modification, or control of primary, regional, or metastatic cancer tissue, or the reduction or amelioration of the progression, severity, and/or duration of a disorder associated with aberrant expression and/or activity of GPNMB or amelioration of one or more symptoms thereof resulting from the administration of one or more therapies. In certain embodiments, such terms in the context of cancer refer to a reduction in the growth of cancerous cells, a decrease in number of cancerous cells and/or a reduction in the growth, formation and/or volume of a tumor. In other embodiments, such terms refer to the minimizing or delay of the spread of cancer resulting from the administration of one or more therapies to a subject with such a disease. Treatment can include, for example, a decrease in the severity of a sypmtopm, the number of symptoms, or frequency of relapse.

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. (See e.g., Sambrook et al. Molecular Cloning: A Laboratory Manual, 2d ed., Cold Spring Harbor Laboratory Press, Cold Spring Harbor, N.Y. 1989). The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

### Antibodies

Antibodies, also known as immunoglobulins, are typically tetrameric glycosylated proteins composed of two light (L) chains (about 25 kDa) and two heavy (H) chains (about 50-70 kDa). The amino-terminal portion of each chain includes a variable domain of about 100 to 110 or more amino acids primarily responsible for antigen recognition. The carboxy-terminal portion of the L and H chain has one and three or four constant domains, respectively that are primarily responsible for effector function. There are two types of human L chains, classified as kappa and lambda. H chains are classified as mu, delta, gamma, alpha, or epsilon based upon the constant domain amino acid sequence, defining the antibody's isotype as IgM, IgD, IgG, IgA, and IgE, respectively. Isotypes may be further divided into subclasses e.g. IgG₁, IgG₂, IgG₃, IgG₄.

Immunoglobulins can be produced naturally in vivo by B lymphocytes. Each clone of B cells produces antibody with an antigen receptor having a unique prospective antigen binding structure. The repertoire of antigen receptors, approximately 10⁷ possibilities, exists in vivo prior to antigen stimulation. This diversity is produced by somatic recombination, i.e., the joining of different antibody gene segments. Immunoglobulin H chain, kappa L chain and lambda L chain are encoded by three separate genetic loci and each locus has multiple copies of at least 3 types of gene segments encoding variable (V), constant (C) and joining (J) regions, the heavy chain gene also includes a diversity (D) region. The selection of specific V, C and J regions (and D for the heavy chain) from amongst the various gene segments available (45 heavy chain V; 35 kappa V; 23 heavy chain D; 6 heavy chain J; 5 kappa J) generates approximately 10¹¹ possible specificities of germline sequences exhibited in B cells. The joining of V, C and J regions can result in the loss or addition of residues at the junctions. The L and H chain V region of human antibodies consists of relatively conserved framework regions (FR) that form a scaffold for three hypervariable regions also known as complementary determining regions (CDR). From the amino terminus of either the heavy or light chain, the V domain is made up of FR and CDR regions in the following order: FR1-CDR1-FR2-CDR2-FR3. Joining of the V domain with a D (heavy chain only) and J domain adds CDR3-FR4. The CDRs are generally responsible for antigen binding.

The specificity of monoclonal antibodies have made them attractive agents for targeting cancer in vivo with the hopes of eradicating disease while sparing normal tissue. The approach, which initially utilized mouse monoclonal antibodies has encountered limitations to potential effectiveness such as immunogenicity; inefficient effector functions and short half-life in vivo. Technologies were developed for: chimeric antibodies which sought to utilize the antigen binding variable domains of mouse monoclonal antibodies combined with the constant regions of human antibodies (Boulianne, et al. 1984 Nature 312:643-646; Morrison et al, 1984 PNAS USA 81:6851-6855); humanized antibodies which grafted antigen binding complementary determining regions (CDRs) from mouse antibodies to human immunoglobulin (Jones, et al, 1986 Nature 321: 522-525; Riechmann, et al, 1988 Nature 332:323-327; Verhoeyen, et al, 1988 Science 239:1534-1536; Vaughan, et al, 1998 Nature Biotechnol. 16:535-539); and phage display libraries of single chain scFvs or Fab fragments of antibodies (de Haard, et al, 1999 J. Biol. Chem. 274: 18218-18230; Knappik, et al, 2000 J. Mol. Biol. 296:57-86; Sheets, et al, 1998 PNAS USA 95:6157-6162; Vaughan, et al, 1994 Nature Biotechnol 14:309-314, 1996; Griffiths et al EMBO J. 13:3245-3260). Additionally, transgenic animals having human immunoglobulin genes and nonfunctional endogenous genes have been developed for immunization and production of fully human monoclonal antibodies (Fishwild, et al, 1996 Nature Biotechnol 14:845-851; Mendez, et al, 1997 Nature Genet. 15:146-156; Nicholson, et al, 1999 J. Immunol. 163, 6898-6906).

Single Chain Antibodies:

Single chain Fv antibodies (scFvs) were first described in the late 1980's (Bird et al., Science 242:423-426 (1988); Huston et al., Proc. Natl. Acad. Sci. USA 85:5879-5883 (1988)). A polypeptide linker, typically ranging in length from 5 to 27 amino acid residues, is used to join the C-terminus of the variable light chain domain (V_{L}) to the N-terminus of the variable heavy chain domain (V_{H}). Alternatively, the linker joins the C-terminus of the V_{H} to the N-terminus of the V_{L}. Both formats (V_{L}-V_{H} and V_{H}-V_{L}) have been used successfully in the literature. The most common linker used in the literature is the (Gly₄Ser)₃ 15 amino acid linker, however there are several other linkers that have been utilized, including a 25 amino acid linker called 205C (Pantoliano et al., Biochemistry 30:10117-10125 (1991)). Single chain antibodies are currently in the clinic; one of the most advanced is h5G1.1 or Pexelizumab. This scFv is specific for human C5 complement and is being used in clinical trials for cardiac patients undergoing cardiopulmonary bypass surgery (Shernan et al., Ann. Thorac Surg. 77:942-949 (2004)).

Bispecific Antibodies (bi-Abs):

An area of mAb research where considerable progress has been made is in the development of bispecific antibodies (biAbs). There are distinct advantages to developing therapeutic antibody molecules with dual specificity. For example, biAbs can serve as mediators to target immune effector cells such as CTLs to unwanted cells (Baeuerle et al., Curr. Opin. Mol. Ther. 5:413-419 (2003)). In another example, chemically linked bispecific antibodies directed against Fc gamma receptors CD16, CD64, and CD89, were significantly more effective in vitro than conventional IgG antibodies (Peipp and Valerius, Biochem. Soc. Trans. 30:507-511 (2002)). One of the challenges in developing biAbs as viable therapeutics has been producing large enough quantities of a stable moiety for clinical applications. Another challenge has been in determining the right combination of validated targets and the underlying biology that would lead to a therapeutic product. For recent reviews on the difficulties experienced with biAbs, see (Kontermann, Acta Pharmacol Sin 26:1-9 (2005); Peipp and Valerius, Soc. Trans. 30:507-511 (2002)).

Bispecific Single Chain Antibodies (bi-scFv):

A notable type of biAb that can be made is a bi-specific single chain antibody or bi-scFv. For a review on the generation of bi-scFv's see (Kipriyanov and Le Gall, Curr Opin Drug Discov Devel 7:233-242 (2004)). Bi-scFvs are typically comprised of 4 variable domains, 2 heavy (V_{H}) and 2 light (V_{L}), which are derived from 2 different antibodies. The 4 domains are linked together with 3 short linkers, ranging in length from 5-27 amino acids. The biological activity of this type of antibody depends on several features concerning the construction of the molecule. For example, both the linker sequences between the antibody V domains and the order of the 4 antibody V domains themselves (for the 2 antibodies) can vary, as well as the expression system that is used; all of which can greatly affect the solubility and biological activity of the various resulting products (Kipriyanov et al., J. Mol. Biol. 330:99-111 (2003); Le Gall et al., Protein Eng. Des. Sel. 17:357-366 (2004); Pavlinkova et al., Clin Cancer Res. 5:2613-1619 (1999)).

Cytotoxic T Lymphocytes:

Under normal circumstances, T cells are activated when the CD3/T cell receptor (CD3/TCR) complex binds to a relevant MHC molecule associated with a specific Ag peptide. Engagement of CD3/TCR with MHC results in intracellular signals necessary to trigger an immune response against a pathogen or tumor. Similar signals that cause T cell activation can also be achieved by antibodies that can bind certain structures of the CD3/TCR complex. In the literature, it has been shown that biAbs recognizing both the TCR/CD3 complex and tumor associated antigen (TAA) can trigger the activation program in CTLs in the presence of target cells (Chapoval et al., J. Immunol. 155:1296-1303 (1995)).

Recombinant technologies are being utilized to enable further improvements upon antibody molecules with the goal of enhancing in vivo efficacy. Such technologies provide, for example, for optimizing molecular size, affinity, pharmacokinetics, toxicity, specificity, valency, effector functions, direct and indirect arming, combination therapy, and various prodrug approaches.

The current invention provides germline human antibody heavy chain V, D, J combinations and light chain V, J combinations including nucleotide and amino acid sequence of the V_{H} and V_{L} domain FR and CDR regions with specificity for GPNMB.

Upon exposure to antigen, those B cells with antigen binding specificity based on germline sequences are activated, proliferate, and differentiate to produce immunoglobulins of different isotypes as well as undergo somatic mutation and/or affinity maturation to produce immunoglobulins of higher affinity for the antigen. The current invention provides the nucleotide and amino acid sequence of such affinity matured V domain FR and CDR regions having specificity to GPNMB.

Fab type antibody fragments containing the antigen binding portion of the antibody molecule may consist of the L chain covalently linked by a disulfide bond to a portion of the H chain which has the V domain and first constant domain. Single chain Fv antibody fragment (scFv) has the H variable domain linked to the L variable domain by a polypeptide linker. The invention provides antibody fragments such as Fab and scFv molecules having sequences derived from germline or affinity matured V domains of antibodies binding specifically to GPNMB.

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments (see, e.g., Songsivilai & Lachmann, 1990 Clin. Exp. Immunol. 79: 315-321; Kostelny et al., 1992 J. Immunol. 148:1547-1553). Bispecific antibodies do not exist in the form of fragments having a single binding site (e.g., Fab, Fab′, and Fv).

It will be appreciated that such bifunctional or bispecific antibodies are contemplated and encompassed by the invention. A bispecific single chain antibody with specificity to GPNMB and to the CD3 antigen on cytotoxic T lymphocytes can be used to direct these T cells to tumor cells expressing GPNMB and cause apoptosis and eradication of the tumor. Bispecific scFv constructs for this purpose are described herein. The scFv components specific for GPNMB can be derived from anti-GPNMB antibodies described herein. In some embodiments, the anti-GPNMB antibody components disclosed herein can be used to generate a biologically active scFv directed against GPNMB. The anti-CD3 scFv component of the therapeutic bispecific scFv was derived from a sequence deposited in Genbank (accession number CAE85148). Alternative antibodies known to target CD3 or other T cell antigens may similarly be effective in treating malignancies when coupled with anti-GPNMB, whether on a single-chain backbone or a full IgG.

GPNMB binding human antibodies may include H or L constant domains including L kappa or lambda constant regions, or any isotype H constant domain. In one embodiment of the invention, a human antibody with binding specificity to GPNMB contains germline sequences such as the heavy chain V regions: VH1-2 (SEQ ID NO: 293), VH2-5 (SEQ ID NO: 294), VH3-11 (SEQ ID NO: 295), VH3-21 (SEQ ID NO: 296), VH3-30 (SEQ ID NO:297), VH3-33 (SEQ ID NO: 298), VH4-31 (SEQ ID NO: 299), VH4-59 (SEQ ID NO:300) or VH5-51 (SEQ ID NO:301); the heavy chain D region: D1-20 (amino acid sequences translated by SEQ ID NO: 302), D1-26 (amino acid sequences translated by SEQ ID NO:303), D3-10 (amino acid sequences translated by SEQ ID NO:304), D3-16 (amino acid sequences translated by SEQ ID NO:305), D3-22 (amino acid sequences translated by SEQ ID NO: 306), D3-9 (amino acid sequences translated by SEQ ID NO:307), D4-17 (amino acid sequences translated by SEQ ID NO: 308), D5-24 (amino acid sequences translated by SEQ ID NO: 309), D6-13 (amino acid sequences translated by SEQ ID NO:310), or D6-19 (amino acid sequences translated by SEQ ID NO: 311); the heavy chain J region: JH3b (SEQ ID NO: 312), JH4b (SEQ ID NO:313), JH5b (SEQ ID NO: 314) or JH6b (SEQ ID NO: 315); the light chain V kappa regions A2 (SEQ ID NO:316), A3 (SEQ ID NO: 317), A20 (SEQ ID NO: 318), A27 (SEQ ID NO: 319), A30 (SEQ ID NO:320), L2 (SEQ ID NO:321) or O1 (SEQ ID NO: 322); and the J region JK1 (SEQ ID NO:323), JK2 (SEQ ID NO: 324), JK3 (SEQ ID NO: 325), JK4 (SEQ ID NO: 326) or JK5 (SEQ ID NO: 327). (generally, see Kabat Sequences of Proteins of Immunological Interest, National Institutes of Health, Bethesda, Md. 1987 and 1991; also see Chothia & Lesk 1987 J. Mol. Biol. 196:901-917; Chothia et al. 1989 Nature 342:878-883).

| | SEQ | |
|---|---|---|
| Germine | ID | |
| sequence | NO: | Sequence |
| VH1-2 | 311 | Gln Val Gln Leu Val Gln Ser Gly Ala |
| | | Glu Val Lys Lys Pro Gly Ala Ser Val |
| | | Lys Val Ser Cys Lys Ala Ser Gly Tyr |
| | | Thr Phe Thr Gly Tyr Tyr Met His Trp |
| | | Val Arg Gln Ala Pro Gly Gln Gly Leu |
| | | Glu Trp Met Gly Trp Ile Asn Pro Asn |
| | | Ser Gly Gly Thr Asn Tyr Ala Gln Lys |
| | | Phe Gln Gly Arg Val Thr Met Thr Arg |
| | | Asp Thr Ser Ile Ser Thr Ala Tyr Met |
| | | Glu Leu Ser Arg Leu Arg Ser Asp Asp |
| | | Thr Ala Val Tyr Tyr Cys Ala Arg |
| VH2-5 | 312 | Gln Val Gln Leu Val Gln Ser Gly Ala |
| | | Glu Val Lys Lys Pro Gly Ala Ser Val |
| | | Lys Val Ser Cys Lys Ala Ser Gly Tyr |
| | | Thr Phe Thr Gly Tyr Tyr Met His Trp |
| | | Val Arg Gln Ala Pro Gly Gln Gly Leu |
| | | Glu Trp Met Gly Trp Ile Asn Pro Asn |
| | | Ser Gly Gly Thr Asn Tyr Ala Gln Lys |
| | | Phe Gln Gly Arg Val Thr Met Thr Arg |
| | | Asp Thr Ser Ile Ser Thr Ala Tyr Met |
| | | Glu Leu Ser Arg Leu Arg Ser Asp Asp |
| | | Thr Ala Val Tyr Tyr Cys Ala Arg |
| VH3-11 | 313 | Gln Val Gln Leu Val Glu Ser Gly Gly |
| | | Gly Leu Val Lys Pro Gly Gly Ser Leu |
| | | Arg Leu Ser Cys Ala Ala Ser Gly Phe |
| | | Thr Phe Ser Asp Tyr Tyr Met Ser Trp |
| | | Ile Arg Gln Ala Pro Gly Lys Gly Leu |
| | | Glu Trp Val Ser Tyr Ile Ser Ser Ser |
| | | Gly Ser Thr Ile Tyr Tyr Ala Asp Ser |
| | | Val Lys Gly Arg Phe Thr Ile Ser Arg |
| | | Asp Asn Ala Lys Asn Ser Leu Tyr Leu |
| | | Gln Met Asn Ser Leu Arg Ala Glu Asp |
| | | Thr Ala Val Tyr Tyr Cys Ala Arg |
| VH3-21 | 314 | Glu Val Gln Leu Val Glu Ser Gly Gly |
| | | Gly Leu Val Lys Pro Gly Gly Ser Leu |
| | | Arg Leu Ser Cys Ala Ala Ser Gly Phe |
| | | Thr Phe Ser Ser Tyr Ser Met Asn Trp |
| | | Val Arg Gln Ala Pro Gly Lys Gly Leu |
| | | Glu Trp Val Ser Ser Ile Ser Ser Ser |
| | | Ser Ser Tyr Ile Tyr Tyr Ala Asp Ser |
| | | Val Lys Gly Arg Phe Thr Ile Ser Arg |
| | | Asp Asn Ala Lys Asn Ser Leu Tyr Leu |
| | | Gln Met Asn Ser Leu Arg Ala Glu Asp |
| | | Thr Ala Val Tyr Tyr Cys Ala Arg |
| VH3-30 | 315 | Gln Val Gln Leu Val Glu Ser Gly Gly |
| | | Gly Val Val Gln Pro Gly Arg Ser Leu |
| | | Arg Leu Ser Cys Ala Ala Ser Gly Phe |
| | | Thr Phe Ser Ser Tyr Gly Met His Trp |
| | | Val Arg Gln Ala Pro Gly Lys Gly Leu |
| | | Glu Trp Val Ala Val Ile Ser Tyr Asp |
| | | Gly Ser Asn Lys Tyr Tyr Ala Asp Ser |
| | | Val Lys Gly Arg Phe Thr Ile Ser Arg |
| | | Asp Asn Ser Lys Asn Thr Leu Tyr Leu |
| | | Gln Met Asn Ser Leu Arg Ala Glu Asp |
| | | Thr Ala Val Tyr Tyr Cys Ala Lys |
| VH3-33 | 316 | Gln Val Gln Leu Val Glu Ser Gly Gly |
| | | Gly Val Val Gln Pro Gly Arg Ser Leu |
| | | Arg Leu Ser Cys Ala Ala Ser Gly Phe |
| | | Thr Phe Ser Ser Tyr Gly Met His Trp |
| | | Val Arg Gln Ala Pro Gly Lys Gly Leu |
| | | Glu Trp Val Ala Val Ile Trp Tyr Asp |
| | | Gly Ser Asn Lys Tyr Tyr Ala Asp Ser |
| | | Val Lys Gly Arg Phe Thr Ile Ser Arg |
| | | Asp Asn Ser Lys Asn Thr Leu Tyr Leu |
| | | Gln Met Asn Ser Leu Arg Ala Glu Asp |
| | | Thr Ala Val Tyr Tyr Cys Ala Arg |
| VH4-31 | 317 | Gln Val Gln Leu Gln Glu Ser Gly Pro |
| | | Gly Leu Val Lys Pro Ser Gln Thr Leu |
| | | Ser Leu Thr Cys Thr Val Ser Gly Gly |
| | | Ser Ile Ser Ser Gly Gly Tyr Tyr Trp |
| | | Ser Trp Ile Arg Gln His Pro Gly Lys |
| | | Gly Leu Glu Trp Ile Gly Tyr Ile Tyr |
| | | Tyr Ser Gly Ser Thr Tyr Tyr Asn Pro |
| | | Ser Leu Lys Ser Arg Val Thr Ile Ser |
| | | Val Asp Thr Ser Lys Asn Gln Phe Ser |
| | | Leu Lys Leu Ser Ser Val Thr Ala Ala |
| | | Asp Thr Ala Val Tyr Tyr Cys Ala Arg |
| VH4-59 | 318 | Gln Val Gln Leu Gln Glu Ser Gly Pro |
| | | Gly Leu Val Lys Pro Ser Glu Thr Leu |
| | | Ser Leu Thr Cys Thr Val Ser Gly Gly |
| | | Ser Ile Ser Ser Tyr Tyr Trp Ser Trp |
| | | Ile Arg Gln Pro Pro Gly Lys Gly Leu |
| | | Glu Trp Ile Gly Tyr Ile Tyr Tyr Ser |
| | | Gly Ser Thr Asn Tyr Asn Pro Ser Leu |
| | | Lys Ser Arg Val Thr Ile Ser Val Asp |
| | | Thr Ser Lys Asn Gln Phe Ser Leu Lys |
| | | Leu Ser Ser Val Thr Ala Ala Asp Thr |
| | | Ala Val Tyr Tyr Cys Ala Arg |
| VH5-51 | 319 | Glu Val Gln Leu Val Gln Ser Gly Ala |
| | | Glu Val Lys Lys Pro Gly Glu Ser Leu |
| | | Lys Ile Ser Cys Lys Gly Ser Gly Tyr |
| | | Ser Phe Thr Ser Tyr Trp Ile Gly Trp |
| | | Val Arg Gln Met Pro Gly Lys Gly Leu |
| | | Glu Trp Met Gly Ile Ile Tyr Pro Gly |
| | | Asp Ser Asp Thr Arg Tyr Ser Pro Ser |
| | | Phe Gln Gly Gln Val Thr Ile Ser Ala |
| | | Asp Lys Ser Ile Ser Thr Ala Tyr Leu |
| | | Gln Trp Ser Ser Leu Lys Ala Ser Asp |
| | | Thr Ala Met Tyr Tyr Cys Ala Arg |
| D1-20 | 320 | ggtataactg gaacgac |
| D1-26 | 321 | ggtatagtgg gagctactac |
| D3-10 | 322 | gtattactat ggttcgggga gttattataa c |
| D3-16 | 323 | gtattatgat tacgtttggg ggagttatcg |
| | | ttatacc |
| D3-22 | 324 | gtattactat gatagtagtg gttattacta c |
| D3-9 | 325 | gtattacgat attttgactg gttattataa c |
| D4-17 | 326 | tgactacggt gactac |
| D5-24 | 327 | gtagagatgg ctacaattac |
| D6-13 | 328 | gggtatagca gcagctggta c |
| D6-19 | 329 | gggtatagca gtggctggta c |
| JH3b | 330 | Ala Phe Asp Ile Trp Gly Gln Gly Thr |
| | | Met Val Thr Val Ser Ser |
| JH4b | 331 | Tyr Phe Asp Tyr Trp Gly Gln Gly Thr |
| | | Leu Val Thr Val Ser Ser |
| JH5b | 332 | Asn Trp Phe Asp Pro Trp Gly Gln Gly |
| | | Thr Leu Val Thr Val Ser Ser |
| JH6b | 333 | Tyr Tyr Tyr Tyr Tyr Gly Met Asp Val |
| | | Trp Gly Gln Gly Thr Thr Val Thr Val |
| | | Ser Ser |
| A2 | 334 | Asp Ile Val Met Thr Gln Thr Pro Leu |
| | | Ser Leu Ser Val Thr Pro Gly Gln Pro |
| | | Ala Ser Ile Ser Cys Lys Ser Ser Gln |
| | | Ser Leu Leu His Ser Asp Gly Lys Thr |
| | | Tyr Leu Tyr Trp Tyr Leu Gln Lys Pro |
| | | Gly Gln Pro Pro Gln Leu Leu Ile Tyr |
| | | Glu Val Ser Asn Arg Phe Ser Gly Val |
| | | Pro Asp Arg Phe Ser Gly Ser Gly Ser |
| | | Gly Thr Asp Phe Thr Leu Lys Ile Ser |
| | | Arg Val Glu Ala Glu Asp Val Gly Val |
| | | Tyr Tyr Cys Met Gln Ser Ile Gln Leu |
| | | Pro |
| A3 | 335 | Asp Ile Val Met Thr Gln Ser Pro Leu |
| | | Ser Leu Pro Val Thr Pro Gly Glu Pro |
| | | Ala Ser Ile Ser Cys Arg Ser Ser Gln |
| | | Ser Leu Leu His Ser Asn Gly Tyr Asn |
| | | Tyr Leu Asp Trp Tyr Leu Gln Lys Pro |
| | | Gly Gln Ser Pro Gln Leu Leu Ile Tyr |
| | | Leu Gly Ser Asn Arg Ala Ser Gly Val |
| | | Pro Asp Arg Phe Ser Gly Ser Gly Ser |
| | | Gly Thr Asp Phe Thr Leu Lys Ile Ser |
| | | Arg Val Glu Ala Glu Asp Val Gly Val |
| | | Tyr Tyr Cys Met Gln Ala Leu Gln Thr |
| | | Pro |
| A20 | 336 | Asp Ile Gln Met Thr Gln Ser Pro Ser |
| | | Ser Leu Ser Ala Ser Val Gly Asp Arg |
| | | Val Thr Ile Thr Cys Arg Ala Ser Gln |
| | | Gly Ile Ser Asn Tyr Leu Ala Trp Tyr |
| | | Gln Gln Lys Pro Gly Lys Val Pro Lys |
| | | Leu Leu Ile Tyr Ala Ala Ser Thr Leu |
| | | Gln Ser Gly Val Pro Ser Arg Phe Ser |
| | | Gly Ser Gly Ser Gly Thr Asp Phe Thr |
| | | Leu Thr Ile Ser Ser Leu Gln Pro Glu |
| | | Asp Val Ala Thr Tyr Tyr Cys Gln Lys |
| | | Tyr Asn Ser Ala Pro |
| A27 | 337 | Glu Ile Val Leu Thr Gln Ser Pro Gly |
| | | Thr Leu Ser Leu Ser Pro Gly Glu Arg |
| | | Ala Thr Leu Ser Cys Arg Ala Ser Gln |
| | | Ser Val Ser Ser Ser Tyr Leu Ala Trp |
| | | Tyr Gln Gln Lys Pro Gly Gln Ala Pro |
| | | Arg Leu Leu Ile Tyr Gly Ala Ser Ser |
| | | Arg Ala Thr Gly Ile Pro Asp Arg Phe |
| | | Ser Gly Ser Gly Ser Gly Thr Asp Phe |
| | | Thr Leu Thr Ile Ser Arg Leu Glu Pro |
| | | Glu Asp Phe Ala Val Tyr Tyr Cys Gln |
| | | Gln Tyr Gly Ser Ser Pro |
| A30 | 338 | Asp Ile Gln Met Thr Gln Ser Pro Ser |
| | | Ser Leu Ser Ala Ser Val Gly Asp Arg |
| | | Val Thr Ile Thr Cys Arg Ala Ser Gln |
| | | Gly Ile Arg Asn Asp Leu Gly Trp Tyr |
| | | Gln Gln Lys Pro Gly Lys Ala Pro Lys |
| | | Arg Leu Ile Tyr Ala Ala Ser Ser Leu |
| | | Gln Ser Gly Val Pro Ser Arg Phe Ser |
| | | Gly Ser Gly Ser Gly Thr Glu Phe Thr |
| | | Leu Thr Ile Ser Ser Leu Gln Pro Glu |
| | | Asp Phe Ala Thr Tyr Tyr Cys Leu Gln |
| | | His Asn Ser Tyr Pro |
| L2 | 339 | Glu Ile Val Met Thr Gln Ser Pro Ala |
| | | Thr Leu Ser Val Ser Pro Gly Glu Arg |
| | | Ala Thr Leu Ser Cys Arg Ala Ser Gln |
| | | Ser Val Ser Ser Asn Leu Ala Trp Tyr |
| | | Gln Gln Lys Pro Gly Gln Ala Pro Arg |
| | | Leu Leu Ile Tyr Gly Ala Ser Thr Arg |
| | | Ala Thr Gly Ile Pro Ala Arg Phe Ser |
| | | Gly Ser Gly Ser Gly Thr Glu Phe Thr |
| | | Leu Thr Ile Ser Ser Leu Gln Ser Glu |
| | | Asp Phe Ala Val Tyr Tyr Cys Gln Gln |
| | | Tyr Asn Asn Trp Pro |
| O1 | 340 | Asp Ile Val Met Thr Gln Thr Pro Leu |
| | | Ser Leu Pro Val Thr Pro Gly Glu Pro |
| | | Ala Ser Ile Ser Cys Arg Ser Ser Gln |
| | | Ser Leu Leu Asp Ser Asp Asp Gly Asn |
| | | Thr Tyr Leu Asp Trp Tyr Leu Gln Lys |
| | | Pro Gly Gln Ser Pro Gln Leu Leu Ile |
| | | Tyr Thr Leu Ser Tyr Arg Ala Ser Gly |
| | | Val Pro Asp Arg Phe Ser Gly Ser Gly |
| | | Ser Gly Thr Asp Phe Thr Leu Lys Ile |
| | | Ser Arg Val Glu Ala Glu Asp Val Gly |
| | | Val Tyr Tyr Cys Met Gln Arg Ile Glu |
| | | Phe Pro |
| JK1 | 341 | Trp Thr Phe Gly Gln Gly Thr Lys Val |
| | | Glu Ile Lys |
| JK2 | 342 | Tyr Thr Phe Gly Gln Gly Thr Lys Leu |
| | | Glu Ile Lys |
| JK3 | 343 | Phe Thr Phe Gly Pro Gly Thr Lys Val |
| | | Asp Ile Lys |
| JK4 | 344 | Leu Thr Phe Gly Gly Gly Thr Lys Val |
| | | Glu Ile Lys |
| JK5 | 345 | Ile Thr Phe Gly Gln Gly Thr Arg Leu |
| | | Glu Ile Lys |

In a particular embodiment of the invention human antibodies with binding specificity to GPNMB are combined germline regions as shown in Table 1.

TABLE 1
Human anti-GPNMB antibody germline region combinations.

| Ab | VH | D | JH | VL | JL |
|---|---|---|---|---|---|
| 1.10.2 | VH4-59 | D6-19 | JH4b | A3 | JK5 |
| 1.15.1 | VH4-31 | D1-20 | JH4b | L2 | JK1 |
| 1.2.2 | VH2-5 | D3-16 | JH4b | O1 | JK5 |
| 1.7.1 | VH4-31 | D1-20 | JH4b | L2 | JK1 |
| 2.10.2 | VH3-30 | D3-10 | JH6b | A3 | JK5 |
| 2.15.1 | VH3-33 | D4-17 | JH4b | A20 | JK4 |
| 2.16.1 | VH3-11 | D6-13 | JH3b | L2 | JK3 |
| 2.17.1 | VH1-2 | D6-19 | JH5b | A2 | JK4 |
| 2.21.2 | VH3-21 | D1-26 | JH4b | A20 | JK5 |
| 2.22.1 | VH4-31 | D3-22 | JH6b | A30 | JK1 |
| 2.24.1 | VH5-51 | D5-24 | JH4b | A27 | JK1 |
| 2.3.1 | VH1-2 | D3-10 | JH4b | A2 | JK4 |
| 2.7.1 | VH3-33 | D3-10 | JH4b | A20 | JK4 |
| 2.8.1 | VH2-5 | D3-9 | JH4b | O1 | JK4 |

In an embodiment of the invention, the isolated antibody has a heavy chain variable region polypeptide comprising an amino acid sequence selected from the group consisting of SEQ ID NOs:2, 20, 38, 56, 74, 92, 110, 128, 146, 164, 182, 200, 218, 236, 253, 256, 260, 265, 270, 274, 277, 281 and 285. Such amino acid sequences can be encoded by nucleotide sequences selected from the group consisting of SEQ ID NOs: 1, 19, 37, 55, 73, 91, 109, 127, 145, 163, 181, 199, 217 and 235. In another embodiment, the invention provides an isolated antibody that specifically binds to GPNMB and has a light chain variable region polypeptide comprising an amino acid sequence selected from the group consisting of SEQ ID NOs: 11, 29, 47, 65, 83, 101, 119, 137, 155, 173, 191, 209, 227 and 245. Such amino acid sequences can be encoded by nucleotide sequences selected from the group consisting of SEQ ID NOs: 10, 28, 46, 64, 82, 100, 118, 136, 154, 172, 190, 208, 226 and 244. In yet another embodiment, the invention provides an isolated antibody that specifically binds to GPNMB and has a heavy chain polypeptide comprising an amino acid sequence selected from the group consisting of SEQ ID NOs: 2, 20, 38, 56, 74, 92, 110, 128, 146, 164, 182, 200, 218, 236, 253, 256, 260, 265, 270, 274, 277, 281 and 285 and has a light chain polypeptide comprising an amino acid sequence selected from the group consisting of SEQ ID NOs: 11, 29, 47, 65, 83, 101, 119, 137, 155, 173, 191, 209, 227 and 245. In yet another embodiment of the invention, anti-GPNMB antibodies comprise at least one CDR of any of the H or L CDR polypeptide sequences SEQ ID NOs: 4, 6, 8, 13, 15, 17, 22, 24, 26, 31, 33, 35, 40, 42, 44, 49, 51, 53, 58, 60, 62, 67, 69, 71, 76, 78, 80, 85, 87, 89, 94, 96, 98, 103, 105, 107, 112, 114, 116, 121, 123, 125, 130, 132, 134, 139, 141, 143, 148, 150, 152, 157, 159, 161, 166, 168, 170, 175, 177, 179, 184, 186, 188, 193, 195, 197, 202, 204, 206, 211, 213, 215, 220, 222, 224, 229, 231, 233, 238, 240, 242, 247, 249, 251, 254, 257, 261, 266, 271, 278, 282, 286, 255, 258, 262, 267, 272, 275, 279, 283, 287, 259, 263, 264, 268, 269, 273, 276, 280, 284 and 288.

In particular embodiments, human anti-GPNMB antibodies are Mab1.10.2, Mab1.15.1, Mab1.2.2, Mab1.7.1, Mab2.10.2, Mab2.15.1, Mab2.16.1, Mab2.17.1, Mab2.21.2, Mab2.22.1, Mab2.24.1, Mab2.3.1, Mab2.7.1, and Mab2.8.1. These antibodies have amino acid sequences and nucleic acid sequences encoding them identified in this application as shown in Tables 2A-2D.

TABLE 2A
Antibody Nucleotide (DNA) and Amino Acid (AA) Sequences

| Gene | | | | |
|---|---|---|---|---|
| Seg- | | | | |
| ment | 1.10.2 | 1.15.1 | 1.2.2 | 1.7.1 |
| H | SEQ ID NO: | SEQ ID NO: 19 | SEQ ID NO: 37 | SEQ ID NO: |
| variable | 1 | | | 55 |
| DNA | | | | |
| H | SEQ ID NO: | SEQ ID NO: 20 | SEQ ID NO: 38 | SEQ ID NO: |
| variable | 2 | | | 56 |
| AA | | | | |
| H FRI | SEQ ID NO: | SEQ ID NO: 21 | SEQ ID NO: 39 | SEQ ID NO: |
| | 3 | | | 57 |
| H CDR1 | SEQ ID NO: | SEQ ID NO: 22 | SEQ ID NO: 40 | SEQ ID NO: |
| | 4 | | | 58 |
| H FR2 | SEQ ID NO: | SEQ ID NO: 23 | SEQ ID NO: 41 | SEQ ID NO: |
| | 5 | | | 59 |
| H CDR2 | SEQ ID NO: | SEQ ID NO: 24 | SEQ ID NO: 42 | SEQ ID NO: |
| | 6 | | | 60 |
| H FR3 | SEQ ID NO: | SEQ ID NO: 25 | SEQ ID NO: 43 | SEQ ID NO: |
| | 7 | | | 61 |
| H CDR3 | SEQ ID NO: | SEQ ID NO: 26 | SEQ ID NO: 44 | SEQ ID NO: |
| | 8 | | | 62 |
| H FR4 | SEQ ID NO: | SEQ ID NO: 27 | SEQ ID NO: 45 | SEQ ID NO: |
| | 9 | | | 63 |
| L | SEQ ID NO: | SEQ ID NO: 28 | SEQ ID NO: 46 | SEQ ID NO: |
| variable | 10 | | | 64 |
| DNA | | | | |
| L | SEQ ID NO: | SEQ ID NO: 29 | SEQ ID NO: 47 | SEQ ID NO: |
| variable | 11 | | | 65 |
| AA | | | | |
| L FR1 | SEQ ID NO: | SEQ ID NO: 30 | SEQ ID NO: 48 | SEQ ID NO: |
| | 12 | | | 66 |
| L CDR1 | SEQ ID NO: | SEQ ID NO: 31 | SEQ ID NO: 49 | SEQ ID NO: |
| | 13 | | | 67 |
| L FR2 | SEQ ID NO: | SEQ ID NO: 32 | SEQ ID NO: 50 | SEQ ID NO: |
| | 14 | | | 68 |
| L CDR2 | SEQ ID NO: | SEQ ID NO: 33 | SEQ ID NO: 51 | SEQ ID NO: |
| | 15 | | | 69 |
| L FR3 | SEQ ID NO: | SEQ ID NO: 34 | SEQ ID NO: 52 | SEQ ID NO: |
| | 16 | | | 70 |
| L CDR3 | SEQ ID NO: | SEQ ID NO: 35 | SEQ ID NO: 53 | SEQ ID NO: |
| | 17 | | | 71 |
| L FR4 | SEQ ID NO: | SEQ ID NO: 36 | SEQ ID NO: 54 | SEQ ID NO: |
| | 18 | | | 72 |

TABLE 2B
Antibody Nucleotide (DNA) and Amino Acid (AA) Sequences

| Gene Segment | 2.10.2 | 2.15.1 | 2.16.1 | 2.17.1 |
|---|---|---|---|---|
| H variable DNA | SEQ ID NO: 73 | SEQ ID NO: 91 | SEQ ID NO: 109 | SEQ ID NO: 127 |
| H variable AA | SEQ ID NO: 74 | SEQ ID NO: 92 | SEQ ID NO: 110 | SEQ ID NO: 128 |
| H FR1 | SEQ ID NO: 75 | SEQ ID NO: 93 | SEQ ID NO: 111 | SEQ ID NO: 129 |
| H CDR1 | SEQ ID NO: 76 | SEQ ID NO: 94 | SEQ ID NO: 112 | SEQ ID NO: 130 |
| H FR2 | SEQ ID NO: 77 | SEQ ID NO: 95 | SEQ ID NO: 113 | SEQ ID NO: 131 |
| H CDR2 | SEQ ID NO: 78 | SEQ ID NO: 96 | SEQ ID NO: 114 | SEQ ID NO: 132 |
| H FR3 | SEQ ID NO: 79 | SEQ ID NO: 97 | SEQ ID NO: 115 | SEQ ID NO: 133 |
| H CDR3 | SEQ ID NO: 80 | SEQ ID NO: 98 | SEQ ID NO: 116 | SEQ ID NO: 134 |
| H FR4 | SEQ ID NO: 81 | SEQ ID NO: 99 | SEQ ID NO: 117 | SEQ ID NO: 135 |
| L variable DNA | SEQ ID NO: 82 | SEQ ID NO: 100 | SEQ ID NO: 118 | SEQ ID NO: 136 |
| L variable AA | SEQ ID NO: 83 | SEQ ID NO: 101 | SEQ ID NO: 119 | SEQ ID NO: 137 |
| L FR1 | SEQ ID NO: 84 | SEQ ID NO: 102 | SEQ ID NO: 120 | SEQ ID NO: 138 |
| L CDR1 | SEQ ID NO: 85 | SEQ ID NO: 103 | SEQ ID NO: 121 | SEQ ID NO: 139 |
| L FR2 | SEQ ID NO: 86 | SEQ ID NO: 104 | SEQ ID NO: 122 | SEQ ID NO: 140 |
| L CDR2 | SEQ ID NO: 87 | SEQ ID NO: 105 | SEQ ID NO: 123 | SEQ ID NO: 141 |
| L FR3 | SEQ ID NO: 88 | SEQ ID NO: 106 | SEQ ID NO: 124 | SEQ ID NO: 142 |
| L CDR3 | SEQ ID NO: 89 | SEQ ID NO: 107 | SEQ ID NO: 125 | SEQ ID NO: 143 |
| L FR4 | SEQ ID NO: 90 | SEQ ID NO: 108 | SEQ ID NO: 126 | SEQ ID NO: 144 |

TABLE 2C
Antibody Nucleotide (DNA) and Amino Acid (AA) Sequences

| Gene Segment | 2.21.2 | 2.22.1 | 2.24.1 | 2.3.1 |
|---|---|---|---|---|
| H variable DNA | SEQ ID NO: 145 | SEQ ID NO: 163 | SEQ ID NO: 181 | SEQ ID NO: 199 |
| H variable AA | SEQ ID NO: 146 | SEQ ID NO: 164 | SEQ ID NO: 182 | SEQ ID NO: 200 |
| H FR1 | SEQ ID NO: 147 | SEQ ID NO: 165 | SEQ ID NO: 183 | SEQ ID NO: 201 |
| H CDR1 | SEQ ID NO: 148 | SEQ ID NO: 166 | SEQ ID NO: 184 | SEQ ID NO: 202 |
| H FR2 | SEQ ID NO: 149 | SEQ ID NO: 167 | SEQ ID NO: 185 | SEQ ID NO: 203 |
| H CDR2 | SEQ ID NO: 150 | SEQ ID NO: 168 | SEQ ID NO: 186 | SEQ ID NO: 204 |
| H FR3 | SEQ ID NO: 151 | SEQ ID NO: 169 | SEQ ID NO: 187 | SEQ ID NO: 205 |
| H CDR3 | SEQ ID NO: 152 | SEQ ID NO: 170 | SEQ ID NO: 188 | SEQ ID NO: 206 |
| H FR4 | SEQ ID NO: 153 | SEQ ID NO: 171 | SEQ ID NO: 189 | SEQ ID NO: 207 |
| L variable DNA | SEQ ID NO: 154 | SEQ ID NO: 172 | SEQ ID NO: 190 | SEQ ID NO: 208 |
| L variable AA | SEQ ID NO: 155 | SEQ ID NO: 173 | SEQ ID NO: 191 | SEQ ID NO: 209 |
| L FR1 | SEQ ID NO: 156 | SEQ ID NO: 174 | SEQ ID NO: 192 | SEQ ID NO: 210 |
| L CDR1 | SEQ ID NO: 157 | SEQ ID NO: 175 | SEQ ID NO: 193 | SEQ ID NO: 211 |
| L FR2 | SEQ ID NO: 158 | SEQ ID NO: 176 | SEQ ID NO: 194 | SEQ ID NO: 212 |
| L CDR2 | SEQ ID NO: 159 | SEQ ID NO: 177 | SEQ ID NO: 195 | SEQ ID NO: 213 |
| L FR3 | SEQ ID NO: 160 | SEQ ID NO: 178 | SEQ ID NO: 196 | SEQ ID NO: 214 |
| L CDR3 | SEQ ID NO: 161 | SEQ ID NO: 179 | SEQ ID NO: 197 | SEQ ID NO: 215 |
| L FR4 | SEQ ID NO: 162 | SEQ ID NO: 180 | SEQ ID NO: 198 | SEQ ID NO: 216 |

TABLE 2D
Antibody Nucleotide (DNA) and Amino Acid (AA) Sequences

| Gene Segment | 2.7.1 | 2.8.1 |
|---|---|---|
| H variable DNA | SEQ ID NO: 217 | SEQ ID NO: 235 |
| H variable AA | SEQ ID NO: 218 | SEQ ID NO: 236 |
| H FR1 | SEQ ID NO: 219 | SEQ ID NO: 237 |
| H CDR1 | SEQ ID NO: 220 | SEQ ID NO: 238 |
| H FR2 | SEQ ID NO: 221 | SEQ ID NO: 239 |
| H CDR2 | SEQ ID NO: 222 | SEQ ID NO: 240 |
| H FR3 | SEQ ID NO: 223 | SEQ ID NO: 241 |
| H CDR3 | SEQ ID NO: 224 | SEQ ID NO: 242 |
| H FR4 | SEQ ID NO: 225 | SEQ ID NO: 243 |
| L variable DNA | SEQ ID NO: 226 | SEQ ID NO: 244 |
| L variable AA | SEQ ID NO: 227 | SEQ ID NO: 245 |
| L FR1 | SEQ ID NO: 228 | SEQ ID NO: 246 |
| L CDR1 | SEQ ID NO: 229 | SEQ ID NO: 247 |
| L FR2 | SEQ ID NO: 230 | SEQ ID NO: 248 |
| L CDR2 | SEQ ID NO: 231 | SEQ ID NO: 249 |
| L FR3 | SEQ ID NO: 232 | SEQ ID NO: 250 |
| L CDR3 | SEQ ID NO: 233 | SEQ ID NO: 251 |
| L FR4 | SEQ ID NO: 234 | SEQ ID NO: 252 |

The variable heavy chains and the variable light chains for various anti-GPNMB antibodies are shown below.

### Antibody-1.10.2

### Heavy Chain Variable Region

Nucleotide sequence
(SEQ ID NO: 1)
5′AGGTGCAGCTGCAGGAGTCGGGCCCAGGACTGGTGAAGCCTTCGG
AGACCCTGTCCCTCACCTGCACTGTCTCTGGTGACTCCATCAGTAAT
TACTACTGGAGCTGGATCCGGCAGCCCCCAGGGAAGGGACTGGAGTG
GATTGGGTATTTCTATTACAGTGGGAGCACCAACTACAACCCCTCCC
TCAAGAGTCGAGTCACCATATCAGTAGACACGTCCAAGAACCAGTTC
TCCCTGAAACTGAGCTCTGTGACCGCTGCGGACACGGCCGTGTATTA
CTGTGCGAGAGATAGGGGCTGGGCTGACTACTGGGGCCAGGGAACCC
TGGTCACCGTCTCCTCAGCC 3′
Amino acid sequence
(SEQ ID NO: 2)
5′QVQLQESGPGLVKPSETLSLTCTVS GDSISNYYWS WIRQPPGK
GLEWIG YFYYSGSTNYNPSLKSRVTISVDTSKNQFSLKLSSVTAAD
TAVYYCAR DRGWADY WGQGTLVTVSSA 3′

TABLE 6
1.10.2 Heavy chain V region domains.

| | | AA | |
|---|---|---|---|
| REGION | SEQUENCE | RESIDUES* | SEQ ID NO: |
| FRI | QVQLQESGPGLVKPSETLSL | 1-25 | SEQ ID NO: |
| | TCTVS | | 3 |
| CDR1 | GDSISNYYWS | 26-35 | SEQ ID NO: |
| | | | 4 |
| FR2 | WIRQPPGKGLEWIG | 36-49 | SEQ ID NO: |
| | | | 5 |
| CDR2 | YFYYSGSTNYNPSLKS | 50-65 | SEQ ID NO: |
| | | | 6 |
| FR3 | RVTISVDTSKNQFSLKLSS | 66-97 | SEQ ID NO: |
| | VTAADTAVYYCAR | | 7 |
| CDR3 | DRGWADY | 98-104 | SEQ ID NO: |
| | | | 8 |
| FR4 | WGQGTLVTVSSA | 105-116 | SEQ ID NO: |
| | | | 9 |

*AA Residues of SEQ ID NO: 2

### Light Chain Variable Region

Nucleotide sequence
(SEQ ID NO: l0)
5′GAAATTGTGTTGACGCAGTCTCCAGGCACCCTGTCTTTGTCTCCA
GGGGAAAGGGCCACCCTCTCCTGCAGAACCAGTCAGAGTATTAGCAG
CAGCTATTTAGCCTGGTACCAGCAGAAACCTGGCCAGGTTCCCAGGC
TCCTCATCTATGGTGCTTCCAGCAGGGCCACTGGCATCCCAGACAGG
TTCAGTGGCAGTGGGTCTGGGACAGACTTCACTCTCACCATCAGCAG
ACTGGAGCCTGAAGATTTTGCAGTGTATTATTGTCAGCAGTATGGTA
GCTCGATCACCTTCGGCCAAGGGACACGACTGGAGATTAAACGA 3′
Amino acid sequence
(SEQ ID NO: 11)
5′EIVLTQSPGTLSLSPGERATLSC RTSQSISSSYLA WYQQKPGQ
VPRLLIY GASSRATGIPDRFSGSGSGTDFTLTISRLEPEDFAVYYC
QQYGSSIT FGQGTRLEIKR 3′

TABLE 7
1.10.2 Light chain V region domains.

| | | AA | |
|---|---|---|---|
| REGION | SEQUENCE | RESIDUES* | SEQ ID |
| FRI | EIVLTQSPGTLSLSP | 1-23 | SEQ ID NO: |
| | GERATLSC | | 12 |
| CDR1 | RTSQSISSSYLA | 24-35 | SEQ ID NO: |
| | | | 13 |
| FR2 | WYQQKPGQVPRLLIY | 36-50 | SEQ ID NO: |
| | | | 14 |
| CDR2 | GASSRAT | 51-57 | SEQ ID NO: |
| | | | 15 |
| FR3 | GIPDRFSGSGSGTDFTL | 58-89 | SEQ ID NO: |
| | TISRLEPEDFAVYYC | | 16 |
| CDR3 | QQYGSSIT | 90-97 | SEQ ID NO: |
| | | | 17 |
| FR4 | FGQGTRLEIKR | 98-108 | SEQ ID NO: |
| | | | 18 |

*AA Residues of SEQ ID NO: 11

### Antibody-1.15.1

### Heavy Chain Variable Region

Nucleotide sequence
(SEQ ID NO: 19)
5′CAGGTGCAGCTGCAGGAGTCGGGCCCAGGACTGGTGAAGCCTTCACAG
ACCCTGTCCCTCACCTGCACTGTCTCTGGTGGCTCCATCAGCAGTTTTAA
TTACTACTGGAGCTGGATCCGCCACCACCCAGGGAAGGGCCTGGAGTGGA
TTGGGTACATCTATTACAGTGGGAGCACCTACTCCAACCCGTCCCTCAAG
AGTCGAGTTACCATATCAGTAGACACGTCTAAGAACCAGTTCTCCCTGAC
GCTGAGCTCTGTGACTGCCGCGGACACGGCCGTGTATTACTGTGCGAGAG
GGTATAACTGGAACTACTTTGACTACTGGGGCCAGGGAACCCTGGTCACC
GTCTCCTCAGCC 3′
Amino acid sequence
(SEQ ID NO: 20)
5′QVQLQESGPGLVKPSQTLSLTCTVSGGSISSFNYYWSWIRHHPGKGLE
WIGYIYYSGSTYSNPSLKSRVTISVDTSKNQFSLTLSSVTAADTAVYYCA
RGYNWNYFDYWGQGTLVTVSSA 3′

TABLE 8
1.15.1 Heavy chain V region domains.

| | | AA | |
|---|---|---|---|
| REGION | SEQUENCE | RESIDUES* | SEQ ID |
| FRI | QVQLQESGPGLVKPSQ | 1-30 | SEQ ID NO: |
| | TLSLTCTVSGGSIS | | 21 |
| CDR1 | SFNYYWS | 31-37 | SEQ ID NO: |
| | | | 22 |
| FR2 | WIRHHPGKGLEWIG | 38-51 | SEQ ID NO: |
| | | | 23 |
| CDR2 | YIYYSGSTYSNPSLKS | 52-67 | SEQ ID NO: |
| | | | 24 |
| FR3 | RVTISVDTSKNQFSLTL | 68-99 | SEQ ID NO: |
| | SSVTAADTAVYYCAR | | 25 |
| CDR3 | GYNWNYFDY | 100-108 | SEQ ID NO: |
| | | | 26 |
| FR4 | WGQGTLVTVSSA | 109-120 | SEQ ID NO: |
| | | | 27 |

*AA Residues of SEQ ID NO: 20

### Light Chain Variable Region

Nucleotide sequence
(SEQ ID NO: 28)
5′GAAATAGTGATGACGCAGTCTCCAGCCACCCTGTCTGTGTCTCCAGG
GGAAAGAGCCACCCTCTCCTGCAGGGCCAGTCAGAGTGTTGACAACAAC
TTAGTCTGGTACCAGCAGAAACCTGGCCAGGCTCCCAGGCTCCTCATCTA
TGGTGCATCCACCAGGGCCACTGGTATCCCAGCCAGGTTCAGTGGCAGTG
GGTCTGGGACAGAGTTCACTCTCACCATCAGTAGTCTGCAGTCTGAAGAT
TTTGCAGTTTATTACTGTCAGCAGTATAATAACTGGCCTCCGTGGACGTT
CGGCCAAGGGACCAAGGTGGAAATCAAACGA 3′
Amino acid sequence
(SEQ ID NO: 29)
5′EIVMTQSPATLSVSPGERATLSCRASQSVDNNLVWYQQKPGQAPRLLIY
GASTRATGIPARFSGSGSGTEFTLTISSLQSEDFAVYYCQQYNNWPPWTFG
QGTKVEIKR 3′

TABLE 9
1.15.1 Light chain V region domains.

| | | AA | |
|---|---|---|---|
| REGION | SEQUENCE | RESIDUES* | SEQ ID |
| FRI | EIVMTQSPATLSVSPGE | 1-23 | SEQ ID NO: |
| | RATLSC | | 30 |
| CDR1 | RASQSVDNNLV | 24-34 | SEQ ID NO: |
| | | | 31 |
| FR2 | WYQQKPGQAPRLLIY | 35-49 | SEQ ID NO: |
| | | | 32 |
| CDR2 | GASTRAT | 50-56 | SEQ ID NO: |
| | | | 33 |
| FR3 | GIPARFSGSGSGTEFTL | 57-88 | SEQ ID NO: |
| | TISSLQSEDFAVYYC | | 34 |
| CDR3 | QQYNNWPPWT | 89-98 | SEQ ID NO: |
| | | | 35 |
| FR4 | FGQGTKVEIKR | 99-109 | SEQ ID NO: |
| | | | 36 |

*AA Residues of SEQ ID NO: 29

### Antibody-1.2.2

### Heavy Chain Variable Region

Nucleotide sequence
(SEQ ID NO: 37)
5′ATCACCTTGAAGGAGTCTGGTCCTACGCTGGTGAAACCCACACAGACCC
TCACGCTGACCTGCACCTTCTCTGGGTTCTCACTCAGCGCTGGTGGAGTGG
GTGTGGGCTGGATCCGTCAGCCCCCAGGAAAGGCCCTGGAGTGGCTTGCAC
TCATTTATTGGAATGATGATAAGCGCTACAGCCCATCTCTGAGGAGCAGGC
TCACCATCACCAAGGACACCTCCAAAAACCAGGTGGTCCTTACAATTACCA
ACATGGACCCTGTGGACACAGCCACATATTATTGTGCACACAGTCACTATG
ATTACGATTGGGGGAGTTACTTTGACTACTGGGGCCAGGGAACCCTGGTCA
CCGTCTCCTCAGCC 3′
Amino acid sequence
(SEQ ID NO: 38)
5′ITLKESGPTLVKPTQTLTLTCTFS GFSLSAGGVGVG WIRQPPG
KALEWLA LIYWNDDKRYSPSLRSRLTITKDTSKNQVVLTITNMDPVDT
ATYYCAHSHYDYDWGSYFDY WGQGTLVTVSSA 3′

TABLE 10
1.2.2 Heavy chain V region domains.

| | | AA | |
|---|---|---|---|
| REGION | SEQUENCE | RESIDUES* | SEQ ID |
| FRI | ITLKESGPTLVKPTQTL | 1-24 | SEQ ID NO: |
| | TLTCTFS | | 39 |
| CDR1 | GFSLSAGGVGVG | 25-36 | SEQ ID NO: |
| | | | 40 |
| FR2 | WIRQPPGKALEWLA | 37-50 | SEQ ID NO: |
| | | | 41 |
| CDR2 | LIYWNDDKRYSPSLRS | 51-66 | SEQ ID NO: |
| | | | 42 |
| FR3 | RLTITKDTSKNQVVLT | 67-98 | SEQ ID NO: |
| | ITNMDPVDTATYYCAH | | 43 |
| CDR3 | SHYDYDWGSYFDY | 99-111 | SEQ ID NO: |
| | | | 44 |
| FR4 | WGQGTLVTVSSA | 112-123 | SEQ ID NO: |
| | | | 45 |

*AA Residues of SEQ ID NO: 38

### Light Chain Variable Region

Nucleotide sequence
(SEQ ID NO: 46)
5′ GATATTGTGATGACCCAGACTCCACTCTCCCTGCCCGTCACC
CCTGGAGAGCCGGCCTCCATCTCCTGCAGGTCTAGTCAGAGCCTC
TTGGATAGTGATGATGGAAACACCTATTTGGACTGGTACCTGCAG
AAGCCAGGACAGTCTCCACAGCTCCTGATCTATACGCTTTCCTAT
CGGGCCTCTGGAGTCCCAGACAGGTTCAGTGGCAGTGGGTCAGGC
ACTGATTTCACACTGAACATCAGCAGGGTGGAGGCTGAGGATGTT
GGAGTTTATTACTGCATGCAACGTATAGAGTTTCCTATCACCTTC
GGCCAAGGGACACGACTGGAGATTAAACGA 3′
Amino acid sequence
(SEQ ID NO: 47)
5′ DIVMTQTPLSLPVTPGEPASISC RSSQSLLDSDDGNTYLD
WYLQKPGQSPQLLIY TLSYRAS GVPDRFSGSGSGTDFTLNIS
RVEAEDVGVYYC MQRIEFPIT FGQGTRLEIKR 3′

TABLE 11
1.2.2 Light chain V region domains.

| | | AA | |
|---|---|---|---|
| REGION | SEQUENCE | RESIDUES* | SEQ ID |
| FRI | DIVMTQTPLSLPVTPGEPASISC | 1-23 | SEQ ID NO: 48 |
| CDR1 | RSSQSLLDSDDGNTYLD | 24-40 | SEQ ID NO: 49 |
| FR2 | WYLQKPGQSPQLLIY | 41-55 | SEQ ID NO: 50 |
| CDR2 | TLSYRAS | 56-62 | SEQ ID NO: 51 |
| FR3 | GVPDRFSGSGSGTDFTLNISRV | 63-94 | SEQ ID NO: 52 |
| | EAEDVGVYYC | | |
| CDR3 | MQRIEFPIT | 95-103 | SEQ ID NO: 53 |
| FR4 | FGQGTRLEIKR | 104-114 | SEQ ID NO: 54 |

*AA Residues of SEQ ID NO: 47

### Antibody-1.7.1

### Heavy Chain Variable Region

Nucleotide sequence
(SEQ ID NO: 55)
5′ CAGGTGCAGCTGCAGGAGTCGGGCCCAGGACTGGTGAA
GCCTTCACAGACCCTGTCCCTCACCTGCACTGTCTCTGGTG
GCTCCATCAGCAGTGCTAATTACTACTGGACCTGGATCCGC
CAGCACCCAGGGAAGGGCCTGGAGTGGATTGGGTACATCTA
TTACAGTGGGAGCACCTACTGCAACCCGTCCCTCAAGAGTC
GAGTTATCATATCAGTAGACACGTCTAAGAACCAGTTCTCC
CTGAAGCTGAGCTCTGTGACTGCCGCGGACACGGCCGTGTA
TTACTGTGCGAGAGGGTATAACTGGAACTACTTTGACTACT
GGGGCCAGGGAACCCTGGTCACCGTCTCCTCAGCC 3′
Amino acid sequence
(SEQ ID NO: 56)
5′ QVQLQESGPGLVKPSQTLSLTCTVS GGSISSANYYWT
WIRQHPGKGLEWIG YIYYSGSTYCNPSLKS
RVIISVDTSKNQFSLKLSSVTAADTAVYYCAR GYNWNYFDY
WGQGTLVTVSSA 3′

TABLE 12
1.7.1 Heavy chain V region domains.

| | | AA | |
|---|---|---|---|
| REGION | SEQUENCE | RESIDUES* | SEQ ID |
| FRI | QVQLQESGPGLVKPSQTLSLTCTV | 1-25 | SEQ ID NO: 57 |
| CDR1 | GGSISSANYYWT | 26-37 | SEQ ID NO: 58 |
| FR2 | WIRQHPGKGLEWIG | 38-51 | SEQ ID NO: 59 |
| CDR2 | YIYYSGSTYCNPSLKS | 52-67 | SEQ ID NO: 60 |
| FR3 | RVIISVDTSKNQFSLKLSSVTAADTAVYYCAR | 68-99 | SEQ ID NO: 61 |
| CDR3 | GYNWNYFDY | 100-108 | SEQ ID NO: 62 |
| FR4 | WGQGTLVTVSSA | 109-120 | SEQ ID NO: 63 |

*AA Residues of SEQ ID NO: 56

### Light Chain Variable Region

Nucleotide sequence
(SEQ ID NO: 64)
5′ GATATAGTGATGACGCAGTCTCCAGCCACCCTGTCTGT
GTCTCCAGGGGAAAGAGCCACCCTCTCCTGCAGGGCCAGTC
AGAGTGTTAGCAGCAACTTAGCCTGGTACCAGGAGAGACCT
GGCCAGGCTCCCAGACTCCTCATCTATGGTGCATCCACCAG
GGCCACTGGTATCCCAGCCAGGTTCAGTGGCAGTGGGTCTG
GGACAGAGTTCACTCTCACCATCAGCAGCCTGCAGTCTGAA
GATTTTGCAGTTTATTACTGTCAGCAGTATAATAAGTGGCC
TCCGTGGACGTTCGGCCAAGGGACCAAGGTGGAAATCGAAC
GAACT 3′
Amino acid sequence
(SEQ ID NO: 65)
5′ DIVMTQSPATLSVSPGERATLSC RASQSVSSNLA
WYQERPGQAPRLLIY GASTRAT GIPARFSGSGSGTEF
TLTISSLQSEDFAVYYC QQYNKWPPWT FGQGTKVEIER 3′

TABLE 13
1.7.1 Light chain V region domains.

| | | AA | |
|---|---|---|---|
| REGION | SEQUENCE | RESIDUES* | SEQ ID |
| FRI | DIVMTQSPATLSVSPGERATLSC | 1-23 | SEQ ID NO: 66 |
| CDR1 | RASQSVSSNLA | 24-34 | SEQ ID NO: 67 |
| FR2 | WYQERPGQAPRLLIY | 35-49 | SEQ ID NO: 68 |
| CDR2 | GASTRAT | 50-56 | SEQ ID NO: 69 |
| FR3 | GIPARFSGSGSGTEFTLTISSLQSEDFAVYYC | 57-88 | SEQ ID NO: 70 |
| CDR3 | QQYNKWPPWT | 89-98 | SEQ ID NO: 71 |
| FR4 | FGQGTKVEIER | 99-109 | SEQ ID NO: 72 |

*AA Residues of SEQ ID NO: 65

### Antibody-2.10.2

### Heavy Chain Variable Region

Nucleotide sequence
(SEQ ID NO: 73)
5′ CAGCTGGTGGAGTCTGGGGGAGGCGTGGTCCAGCCTG
GGAGGTCCCTGAGACTCTCCTGTGCAGCCTCTGGATTCGC
CTTCAGTAGCTATGGCATGCACTGGGTCCGCCAGGCTCCA
GGCAAGGGGCTGGAGTGGGTGGCAGTTATATCATATGATG
GAAATAATAAATACTATGCAGACTCCGTGAAGGGCCGATT
CACCATCTCCAGAGACAATTCCAAGAACACGCTGTATCTG
CAAATGAACAGCCTGAGAGCTGAGGACACGGCTGTGTATT
ACTGTGCGAGAGATCTAGTGGTTCGGGGAATTAGGGGGTA
CTACTACTACTTCGGTATGGACGTCTGGGGCCAAGGGACC
ACGGTCACCGTCTCCTCAGCC 3′
Amino acid sequence
(SEQ ID NO: 74)
5′ QLVESGGGVVQPGRSLRLSCAAS GFAFSSYGMH
WVRQAPGKGLEWVA VISYDGNNKYYADSVKG
RFTISRDNSKNTLYLQMNSLRAEDTAVYYCARDLVVRGIR
GYYYYFGMDV WGQGTTVTVSSA 3′

TABLE 14
2.10.2 Heavy chain V region domains.

| | | AA | |
|---|---|---|---|
| REGION | SEQUENCE | RESIDUES* | SEQ ID |
| FRI | QLVESGGGVVQPGRSLRLSCAAS | 1-23 | SEQ ID NO: 75 |
| CDR1 | GFAFSSYGMH | 24-33 | SEQ ID NO: 76 |
| FR2 | WVRQAPGKGLEWVA | 34-47 | SEQ ID NO: 77 |
| CDR2 | VISYDGNNKYYADSVKG | 48-64 | SEQ ID NO: 78 |
| FR3 | RFTISRDNSKNTLYLQMNSLRAEDTAVYYCAR | 65-96 | SEQ ID NO: 79 |
| CDR3 | DLVVRGIRGYYYYFGMDV | 97-114 | SEQ ID NO: 80 |
| FR4 | WGQGTTVTVSSA | 115-126 | SEQ ID NO: 81 |

*AA Residues of SEQ ID NO: 74

### Light Chain Variable Region

Nucleotide sequence
(SEQ ID NO: 82)
5′ GATATTGTGATGACTCAGTCTCCACTCTCCCTGCCCGTCAC
CCCTGGAGAGCCGGCCTCCATCTCCTGCAGGTCTAGTCAGAGCC
TCCTGCATAGTAATGGATACAACTATTTGGATTGGTACCTGCAG
AAGCCAGGGCAGTCTCCACAGCTCCTGATCTATTTGGGTTCTAA
TCGGGCCTCCGGGGTCCCTGACAGGTTCAGTGGCAGTGGATCAG
GCACAGATTTTACACTGAAAATCAGCAGAGTGGAGGCTGAGGAT
GTTGGGGTTTATTACTGCATGCAAGGTCTACAAACTCCGATCAC
CTTCGGCCAAGGGACACGACTGGAGATTAAACGA 3′
Amino acid sequence
(SEQ ID NO: 83)
5′ DIVMTQSPLSLPVTPGEPASISC RSSQSLLHSNGYNYLD
WYLQKPGQSPQLLIY LGSNRAS GVPDRFSGSGSGTDFTLKI
SRVEAEDVGVYYC MQGLQTPIT FGQGTRLEIKR 3′

TABLE 15
2.10.2 Light chain V region domains.

| | | AA | |
|---|---|---|---|
| REGION | SEQUENCE | RESIDUES* | SEQ ID |
| FRI | DIVMTQSPLSLPVTPGEPASISC | 1-23 | SEQ ID NO: 84 |
| CDR1 | RSSQSLLHSNGYNYLD | 24-39 | SEQ ID NO: 85 |
| FR2 | WYLQKPGQSPQLLIY | 40-54 | SEQ ID NO: 86 |
| CDR2 | LGSNRAS | 55-61 | SEQ ID NO: 87 |
| FR3 | GVPDRFSGSGSGTDFTLKISRVEAEDVGVYYC | 62-93 | SEQ ID NO: 88 |
| CDR3 | MQGLQTPIT | 94-102 | SEQ ID NO: 89 |
| FR4 | FGQGTRLEIKR | 103-113 | SEQ ID NO: 90 |

*AA Residues of SEQ ID NO: 83

### Antibody-2.15.1

### Heavy Chain Variable Region

Nucleotide sequence
(SEQ ID NO: 91)
5′ CAGGTGCAGCTGGTGGAGTCTGGGGGAGGCGTGGTCCA
GCCTGGGAGGTCCCTGAGACTCTCCTGTGCAGCGTCTGGAT
TCACCTTCAGTAACTATGGCATTCACTGGGTCCGCCAGGCT
CCAGGCAAGGGGCTGGAGTGGGTGGCAGTTATATGGTTTGA
TGGACGTAATAAATACTATGCAGACTCCGTGAAGGGCCGAT
TCACCATCTCCAGAGACAATTCCAAGAACACGCTGTATCTG
CAAATGAACAGCCTGAGAGCCGAGGACGCGGCTGTGTATTA
CTGTGCGAGAGATCCCTTTGACTATGGTGACTCCTTCTTTG
ACTACTGGGGCCAGGGCACCCTGGTCACCGTCTCCTCAGCC 3′
Amino acid sequence
(SEQ ID NO: 92)
5′ QVQLVESGGGVVQPGRSLRLSCAAS GFTFSNYGIH
WVRQAPGKGLEWVA VIWFDGRNKYYADSVKG
RFTISRDNSKNTLYLQMNSLRAEDAAVYYCAR DPFDYGDSFFDY
WGQGTLVTVSSA 3′

TABLE 16
2.15.1 Heavy chain V region domains.

AA

| REGION | SEQUENCE | RESIDUES* | SEQ ID |
|---|---|---|---|
| FRI | QVQLVESGGGVVQPGRSLRLSCAAS | 1-25 | SEQ ID NO: 93 |
| CDR1 | GFTFSNYGIH | 26-35 | SEQ ID NO: 94 |
| FR2 | WVRQAPGKGLEWVA | 36-49 | SEQ ID NO: 95 |
| CDR2 | VIWFDGRNKYYADSVKG | 50-66 | SEQ ID NO: 96 |
| FR3 | RFTISRDNSKNTLYLQMNSLRAEDAAVYYCAR | 67-98 | SEQ ID NO: 97 |
| CDR3 | DPFDYGDSFFDY | 99-110 | SEQ ID NO: 98 |
| FR4 | WGQGTLVTVSSA | 111-122 | SEQ ID NO: 99 |

*AA Residues of SEQ ID NO: 92

### Light Chain Variable Region

Nucleotide sequence
(SEQ ID NO: 100)
55′ CTGACTCAGTCTCCATCCTCCCTGTCTGCATCTGTAAG
AGACAGAGTCACCATCACTTGCCGGGCGAGTCAGGACATTAG
CAATTATTTAGCCTGGTATCAGCAGAAACCAGGGAAAGTTCC
TAATCTCCTGATCTATGCTGCATCCACTTTGCAATCAGGGGT
CCCATCTCGGTTCAGTGGCAGTGGATCTGGGACAGATTTCAC
TCTCACCATCAGCAGCCTGCAGCCTGAAGATGTTGCAACTTA
TTACTGTCAAAAGTATAACAGTGCCCCGCTCACTTTCGGCGG
AGGGACCAAGGTGGAGATCAAACGA 3′
Amino acid sequence
(SEQ ID NO: 101)
5′ LTQSPSSLSASVRDRVTITC RASQDISNYLA
WYQQKPGKVPNLLIY AASTLQS GVPSRFSGSGSGTDFTL
TISSLQPEDVATYYC QKYNSAPLT FGGGTKVEIKR 3′

TABLE 17
2.15.1 Light chain V region domains.

| | | AA | |
|---|---|---|---|
| REGION | SEQUENCE | RESIDUES* | SEQ ID |
| FRI | LTQSPSSLSASVRDRVTITC | 1-20 | SEQ ID NO: 102 |
| CDR1 | RASQDISNYLA | 21-31 | SEQ ID NO: 103 |
| FR2 | WYQQKPGKVPNLLIY | 32-46 | SEQ ID NO: 104 |
| CDR2 | AASTLQ | 47-52 | SEQ ID NO: 105 |
| FR3 | GVPSRFSGSGSGTDFTLTISSLQPEDVATYYC | 53-84 | SEQ ID NO: 106 |
| CDR3 | QKYNSAPLT | 85-93 | SEQ ID NO: 107 |
| FR4 | FGGGTKVEIKR | 94-104 | SEQ ID NO: 108 |

*AA Residues of SEQ ID NO: 101

### Antibody-2.16.1

### Heavy Chain Variable Region

Nucleotide sequence
(SEQ ID NO: 109)
5′CAGGTGCAGCTGGTGGAGTCTGGGGGAGGCTTGGTCAAGCC
TGGAGGGTCCCTGAGACTCTCCTGTGCAGCCTCTGGATTCACC
TTCAGTGACTACTACATGACCTGGATCCGCCAGGCTCCAGGGA
AGGGGCTGGAGTGGGTTTCATACATTAGTATTAGTGGTAGTAT
CACACACTACGCAGACTCAGTGAAGGGCCGATTCACCATGTCC
AGGGACAACGCCAAGAACTCACTGTATCTGCAAATGAACAGCC
TGAGAGCCGAGGACACGGCCGTGTATTACTGTGCGAGAGACGG
AGCAGCAGCTGGTACGGATGCTTTTGATATCTGGGGCCACGGG
ACAAAGGTCACCGTCTCTTCAGCC 3′
Amino acid sequence
(SEQ ID NO: 110)
5′ QVQLVESGGGLVKPGGSLRLSCAAS GFTFSDYYMT
WIRQAPGKGLEWVS YISISGSITHYADSVKG RFTMSRDNAKNS
LYLQMNSLRAEDTAVYYCAR DGAAAGTDAFDI WGHGTKVTVSSA 3′

TABLE 18
2.16.1 Heavy chain V region domains.

| | | AA | |
|---|---|---|---|
| REGION | SEQUENCE | RESIDUES* | SEQ ID |
| FRI | QVQLVESGGGLVKPGGSLRLSCAAS | 1-25 | SEQ ID NO: 111 |
| CDR1 | GFTFSDYYMT | 26-35 | SEQ ID NO: 112 |
| FR2 | WIRQAPGKGLEWVS | 36-49 | SEQ ID NO: 113 |
| CDR2 | YISISGSITHYADSVKG | 50-66 | SEQ ID NO: 114 |
| FR3 | RFTMSRDNAKNSLYLQMNSLRAEDT | 67-98 | SEQ ID NO: 115 |
| | AVYYCAR | | |
| CDR3 | DGAAAGTDAFDI | 99-110 | SEQ ID NO: 116 |
| FR4 | WGHGTKVTVSSA | 111-122 | SEQ ID NO: 117 |

*AA Residues of SEQ ID NO: 110

### Light Chain Variable Region

Nucleotide sequence
(SEQ ID NO: 118)
5′GAGATAGTGATGACGCAGTCTCCAGCCACCCTATCTGTGTCTCCA
GGGGACAGAGCCACCCTCTCCTGCAGGGCCAGTCAGAATGTTAGCAG
CAACTTGGCCTGGTACCAGCAGAAACCTGGCCAGGCTCCCAGGCTCC
TCATCTTTGGTGCATCCACCAGGGCCACTGGTATCCCAGCCAGGTTC
AGTGGCAGTGGGTCTGGGACAGAGTTCACTCTCACCATCAGCAGCCT
ACAGTCTGAAGATTTTGCAGTTTATTACTGTCAGCAGTATCATTACT
GGCCCACTTTCGGCCCTGGGACCAAAGTGGATATCAAACGA 3′
Amino acid sequence
(SEQ ID NO: 119)
5′EIVMTQSPATLSVSPGDRATLSC RASQNVSSNLA
WYQQKPGQAPRLLIF GASTRAT GIPARFSGSGSGTEFTLTISSLQ
SEDFAVYYC QQYHYWPT FGPGTKVDIKR 3′

TABLE 19
2.16.1 Light chain V region domains.

| | | AA | |
|---|---|---|---|
| REGION | SEQUENCE | RESIDUES* | SEQ ID |
| FRI | EIVMTQSPATLSVSPGDRATLSC | 1-23 | SEQ ID NO: 120 |
| CDR1 | RASQNVSSNLA | 24-34 | SEQ ID NO: 121 |
| FR2 | WYQQKPGQAPRLLIF | 35-49 | SEQ ID NO: 122 |
| CDR2 | GASTRAT | 50-56 | SEQ ID NO: 123 |
| FR3 | GIPARFSGSGSGTEFTLTISSLQSE | 57-88 | SEQ ID NO: 124 |
| | DFAVYYC | | |
| CDR3 | QQYHYWPT | 89-96 | SEQ ID NO: 125 |
| FR4 | FGPGTKVDIKR | 97-107 | SEQ ID NO: 126 |

*AA Residues of SEQ ID NO: 119

### Antibody-2.17.1

### Heavy Chain Variable Region

Nucleotide sequence
(SEQ ID NO: 127)
5′ CAGCTGGTGCAGTCTGGGGCTGAGGTGAAGAAGCCTGGGGCCTCA
GTGAAGGTCTCCTGCAAGGCTTCTGGATACACCTTCACCGGCTTCTAT
ATGCACTGGGTGCGACAGACCCCTGGACAAGGGCTTGAGTGGATGGGA
TGGATCAACCCTAACAGTGGTGGCACATATTATGTACAGAAGTTTCAG
GGCAGGGTCACCATGACCAGGGACACGTCCATCAGCACAGTCTACATG
GAGCTGAGCAGGTTGAGATCTGACGACACGGCCGTATATTACTGTGCG
AGAGATGGGTATAGCAGTGGAGAGGACTGGTTCGACCCCTGGGGCCAG
GGAACCCTGGTCACCGTCTCCTCAGCC 3′
Amino acid sequence
(SEQ ID NO: 128)
5′ QLVQSGAEVKKPGASVKVSCKAS GYTFTGFYMH
WVRQTPGQGLEWMG WINPNSGGTYYVQKFQG RVTMTRDTSISTVY
MELSRLRSDD TAVYYCAR DGYSSGEDWFDP WGQGTLVTVSSA 3′

TABLE 20
2.17.1 Heavy chain V region domains.

| | | AA | |
|---|---|---|---|
| REGION | SEQUENCE | RESIDUES* | SEQ ID |
| FRI | QLVQSGAEVKKPGASVKVSCKAS | 1-23 | SEQ ID NO: 129 |
| CDR1 | GYTFTGFYMH | 24-33 | SEQ ID NO: 130 |
| FR2 | WVRQTPGQGLEWMG | 34-47 | SEQ ID NO: 131 |
| CDR2 | WINPNSGGTYYVQKFQG | 48-64 | SEQ ID NO: 132 |
| FR3 | RVTMTRDTSISTVYMELSRLRSDD | 65-96 | SEQ ID NO: 133 |
| | TAVYYCAR | | |
| CDR3 | DGYSSGEDWFDP | 97-108 | SEQ ID NO: 134 |
| FR4 | WGQGTLVTVSSA | 109-120 | SEQ ID NO: 135 |

*AA Residues of SEQ ID NO: 128

### Light Chain Variable Region

Nucleotide sequence
(SEQ ID NO: 136)
5′ GATATTGTGATGACCCAGACTCCACTCTCTCTGTCCGTCA
CCCCTGGACAGCCGGCCTCCATCTCCTGCAAGTCTAGTCAGA
GCCTCCTGCATAGTGGTGGAAAGACCTATTTGTATTGGTACC
TGCAGAGGCCAGGCCAGCCTCCACAGCTCCTGATCTATGAAG
TTTCCAACCGGTTCTCTGGAGTGCCAGATAGGTTCAGTGGCA
GCGGGTCAGGGACAGATTTCACACTGAAAATCAGCCGGGTGG
AGGCTGAGGATGTTGGGGTTTATTACTGCATGCAAAGTATAC
ACCTTCCGCTCACTTTCGGCGGAGGGACCAAGGTGGAGATCA
AACGA 3′
Amino acid sequence
(SEQ ID NO: 137)
5′ DIVMTQTPLSLSVTPGQPASISC KSSQSLLHSGGKTYLY
WYLQRPGQPPQLLIY EVSNRFS GVPDRFSGSGSGTDFTL-
KISRVEAEDVGVYYC MQSIHLPLT FGGGTKVEIKR 3′

TABLE 21
2.17.1 Light chain V region domains.

| | | AA | |
|---|---|---|---|
| REGION | SEQUENCE | RESIDUES* | SEQ ID |
| FRI | DIVMTQTPLSLSVTPGQPASISC | 1-23 | SEQ ID NO: 138 |
| CDR1 | KSSQSLLHSGGKTYLY | 24-39 | SEQ ID NO: 139 |
| FR2 | WYLQRPGQPPQLLIY | 40-54 | SEQ ID NO: 140 |
| CDR2 | EVSNRFS | 55-61 | SEQ ID NO: 141 |
| FR3 | GVPDRFSGSGSGTDFTLKISRV | 62-93 | SEQ ID NO: 142 |
| | EAEDVGVYYC | | |
| CDR3 | MQSIFILPLT | 94-102 | SEQ ID NO: 143 |
| FR4 | FGGGTKVEIKR | 103-113 | SEQ ID NO: 144 |

*AA Residues of SEQ ID NO: 137

### Antibody-2.21.1

### Heavy Chain Variable Region

Nucleotide sequence
(SEQ ID NO: 145)
5′ CAGGTGCAGCTGGAGCAGTCGGGGGGAGGCCTGGTCAAGCC
TGGGGGGTCCCTGAGATTCTCCTGTGCAGCCTCTGGATTCACCT
TCAGTAGCTATAGCATGAACTGGGTCCGCCAGGCTCCAGGGAAG
GGGCTGGAGTGGGTCTCATTCATTAGTAGTAGTAGTAGTTACAT
ATACTACGCAGACTCAGTGAAGGGCCGATTCACCATCTCCAGAG
ACAACGCCAAGAACTCACTGTATCTGCAAATGAACAGCCTGAGA
GCCGAGGACACGGCTGTGTATTACTGTGCGAGAGAGGACTGGGT
GGGAGCTACCTTTGACTACTGGGGCCAGGGAACCCTGGTCACCG
TCTCCTCAGCC 3′
Amino acid sequence
(SEQ ID NO: 146)
5′ QVQLEQSGGGLVKPGGSLRFSCAAS GFTFSSYSMN
WVRQAPGKGLEWVS FISSSSSYIYYADSVKG RFTISRDNAK
NSLYLQMNSLRAEDTAVYYCAR EDWVGATFDY WGQGTLVTVSSA 3′

TABLE 22
2.21.1 Heavy chain V region domains.

| | | AA | |
|---|---|---|---|
| REGION | SEQUENCE | RESIDUES* | SEQ ID |
| FRI | QVQLEQSGGGLVKPGGSLRFSCAAS | 1-25 | SEQ ID NO: 147 |
| CDR1 | GFTFSSYSMN | 26-35 | SEQ ID NO: 148 |
| FR2 | WVRQAPGKGLEWVS | 36-49 | SEQ ID NO: 149 |
| CDR2 | FISSSSSYIYYADSVKG | 50-66 | SEQ ID NO: 150 |
| FR3 | RFTISRDNAKNSLYLQMNSLRAEDT | 67-98 | SEQ ID NO: 151 |
| | AVYYCAR | | |
| CDR3 | EDWVGATFDY | 99-108 | SEQ ID NO: 152 |
| FR4 | WGQGTLVTVSSA | 109-120 | SEQ ID NO: 153 |

*AA Residues of SEQ ID NO: 146

### Light Chain Variable Region

Nucleotide sequence
(SEQ ID NO: 154)
5′ GACATTCAGCTGACCCAGTCTCCATCCTCCCTGTCTGCATCT
GTAGGAGACAGAGTCACCATCACTTGTCGGGCGAGTCAGGGCATT
AGGAATTATTTAGCCTGGTATCAGCAGAAACCAGGGAAAGTTCCT
AAGCTCCTGATCTATGCTGCTTCCGCTTTGAAATTAGGGGTCCCA
TCTCGGTTCAGTGGCAGTGGATCTGGGACAGATTTCACTCTCACC
ATCAGCAGCCTGCAGCCTGAAGATGTTGCAACTTATTACTGTCAA
AAGTATAACAGTGCCCCGATCACCTTCGGCCAAGGGACACGACTG
GACATTAAACGA 3′
Amino acid sequence
(SEQ ID NO: 155)
5′ DIQLTQSPSSLSASVGDRVTITC RASQGIRNYLA
WYQQKPGKVPKLLIY AASALKL GVPSRFSGSGSGTDFTLTIS
SLQPEDVATYYC QKYNSAPIT FGQGTRLDIKR 3′

TABLE 23
2.21.1 Light chain V region domains.

| | | AA | |
|---|---|---|---|
| REGION | SEQUENCE | RESIDUES* | SEQ ID |
| FRI | DIQLTQSPSSLSASVGDRVTITC | 1-23 | SEQ ID NO: 156 |
| CDR1 | RASQGIRNYLA | 24-34 | SEQ ID NO: 157 |
| FR2 | WYQQKPGKVPKLLIY | 35-49 | SEQ ID NO: 158 |
| CDR2 | AASALKL | 50-56 | SEQ ID NO: 159 |
| FR3 | GVPSRFSGSGSGTDFTLTISSLQ | 57-88 | SEQ ID NO: 160 |
| | PEDVATYYC | | |
| CDR3 | QKYNSAPIT | 89-97 | SEQ ID NO: 161 |
| FR4 | FGQGTRLDIKR | 98-108 | SEQ ID NO: 162 |

*AA Residues of SEQ ID NO: 155

### Antibody-2.22.1

### Heavy Chain Variable Region

Nucleotide sequence
(SEQ ID NO: 163)
5′ CAGGTGCAGCTGGAGCAGTCGGGCCCAGGACTGGTGAA
GCCTTCACAGAACCTGTCCCTCACCTGCACTGTCTCTGGTG
GCTCCATCAGCAGTGGTGGTTATTTCTGGAGCTGGATCCGC
CAGCACCCAGGGAAGGGCCTGGAGTGGATTGGGTACATCTA
TTACAGTGGGAACACCTACTACAACCCGTCCCTCAAGAGTC
GAGTTACCATATCAGTTGACACGTCTAAGAACCAGTTCTCC
CTGAAACTGAGCTCTGTGACTGCCGCGGACACGGCCGTGTA
TTACTGTGCGAGAGACTATTACTATGATACTAGTGGTTTTT
CCTACCGTTACGACTGGTACTACGGTATGGACGTCTGGGGC
CAAGGGACCACGGTCACCGTCTCCTCAGCC 3′
Amino acid sequence
(SEQ ID NO: 164)
5′ QVQLEQSGPGLVKPSQNLSLTCTVS GGSISSGGYFWS
WIRQHPGKGLEWIG YIYYSGNTYYNPSLKS RVTISVDTSK-
NQFSLKLSSVTAADTAVYYCAR DYYYDTSGFSYRYDWYYGM-
DVWGQGTTVTVSSA 3′

TABLE 24
2.22.1 Heavy chain V region domains.

| | | AA | |
|---|---|---|---|
| REGION | SEQUENCE | RESIDUES* | SEQ ID |
| FRI | QVQLEQSGPGLVKPS | 1-25 | SEQ ID NO: 165 |
| | QNLSLTCTVS | | |
| CDR1 | GGSISSGGYFWS | 26-37 | SEQ ID NO: 166 |
| FR2 | WIRQHPGKGLEWIG | 38-51 | SEQ ID NO: 167 |
| CDR2 | YIYYSGNTYYNPSLKS | 52-67 | SEQ ID NO: 168 |
| FR3 | RVTISVDTSKNQFSLKLS | 68-99 | SEQ ID NO: 169 |
| | SVTAADTAVYYCAR | | |
| CDR3 | DYYYDTSGFSYRYDW | 100-120 | SEQ ID NO: 170 |
| | YYGMDV | | |
| FR4 | WGQGTTVTVSSA | 121-132 | SEQ ID NO: 171 |

*AA Residues of SEQ ID NO: 164

### Light Chain Variable Region

Nucleotide sequence
(SEQ ID NO: 172)
5′GACATCCAGCTGACCCAGTCTCCATCCTCCCTGTCTGCATCTGTAGG
AGACAGAGTCACCATCACTTGCCGGGCAAGTCAGGGCATTAGAAATGAT
TTAGGCTGGTATCAGCAGAAACCAGGGAAAGCCCCTAAGCGCCTGATCT
ATGCTGCATCCAGTTTGCAAAATGGGGTCCCATCAAGGTTCAGCGGCAGT
GGATCTGGGACAGAATTCACTCTCACAATCAGCAGCCTGCAGCCTGAAGA
TTTTGCAACTTATTACTGTCTACAACATAATACTTACCCGGCGTTCGGCC
AAGGGACCAAGGTGGAAATCAAACGA 3′
Amino acid sequence
(SEQ ID NO: 173)
5′DIQLTQSPSSLSASVGDRVTITC RASQGIRNDLG WYQQKPGKAPK
RLIY AASSLQNGVPSRFSGSGSGTEFTLTISSLQPEDFATYYC LQHN
TYPA FGQGTKVEIKR 3′

TABLE 25
2.22.1 Light chain V region domains.

| | | AA | |
|---|---|---|---|
| REGION | SEQUENCE | RESIDUES* | SEQ ID |
| FRI | DIQLTQSPSSLSASVG | 1-23 | SEQ ID NO: 174 |
| | DRVTITC | | |
| CDR1 | RASQGIRNDLG | 24-34 | SEQ ID NO: 175 |
| FR2 | WYQQKPGKAPKRLIY | 35-49 | SEQ ID NO: 176 |
| CDR2 | AASSLQN | 50-56 | SEQ ID NO: 177 |
| FR3 | GVPSRFSGSGSGTEFTLT | 57-88 | SEQ ID NO: 178 |
| | ISSLQPEDFATYYC | | |
| CDR3 | LQHNTYPA | 89-97 | SEQ ID NO: 179 |
| FR4 | FGQGTKVEIKR | 98-108 | SEQ ID NO: 180 |

*AA Residues of SEQ ID NO: 173

### Antibody ˜2.24.1

### Heavy Chain Variable Region

Nucleotide sequence
(SEQ ID NO: 181)
5′CAGCTGGTGCAGTCTGGAGCAGAAGTGAAAAAGCCCGGGG
AGTCTCTGAAGATCTCCTGTCAGGGTTCTGGATACATCTTTA
CCAACTACTGGATCGGCTGGGTGCGCCAGATGCCCGGGAAAG
GCCTGGAGTGGATGGGGGTCATCTATCCTGATGACTCTGATA
CCAGATACAGCCCGTCCTTCCAAGGCCAGGTCACCATCTCAG
CCGACAAGTCCATCAGCACCGCCTACCTGCAGTGGAGCAGCCT
GAAGGCCTCGGACACCGCCATATATTACTGTGCGAGACAAAAA
TGGCTACAACACCCCTTTGACTACTGGGGCCAGGGAACCCTGG
TCACCGTCTCCTCAGCC 3′
Amino acid sequence
(SEQ ID NO: 182)
5′QLVQSGAEVKKPGESLKISCQGS GYIFTNYWIG WVRQMPGK
GLEWMG VIYPDDSDTRYSPSFQGQVTISADKSISTAYLQWSSLK
ASDTAIYYCAR QKWLQHPFDY WGQGTLVTVSSA 3′

TABLE 26
2.24.1 Heavy chain V region domains.

| | | AA | |
|---|---|---|---|
| REGION | SEQUENCE | RESIDUES* | SEQ ID |
| FRI | QLVQSGAEVKKPGESLK | 1-23 | SEQ ID NO: 183 |
| | ISCQGS | | |
| CDR1 | GYIFTNYWIG | 24-33 | SEQ ID NO: 184 |
| FR2 | WVRQMPGKGLEWMG | 34-47 | SEQ ID NO: 185 |
| CDR2 | VIYPDDSDTRYSPSFQG | 48-64 | SEQ ID NO: 186 |
| FR3 | QVTISADKSISTAYLQWSS | 65-96 | SEQ ID NO: 187 |
| | LKASDTAIYYCAR | | |
| CDR3 | QKWLQHPFDY | 97-106 | SEQ ID NO: 188 |
| FR4 | WGQGTLVTVS SA | 107-118 | SEQ ID NO: 189 |

*AA Residues of SEQ ID NO: 182

### Light Chain Variable Region

Nucleotide sequence
(SEQ ID NO: 190)
5′GAAATTGTGTTGACGCAGTCACCAGGCACCCTGTCTTTGTCTCCAGGG
GAAAGAGTCACCCTCTCATGCAGGGCCAGTCAGAGTGTTAGCAGCAGATA
CTTAGCCTGGTACCAGCAGAAACCTGGCCAGGCTCCCAGGCTCCTCATCT
ATGGTGCATCCAGCAGGGCCACTGGCATCCCAGACAGGTTCAGTGGCAGT
GGGTCTGGGACAGACTTCACTCTCACCATCAGCAGACTGGAGCCTGAAGA
TTTTGCAGTTTATTACTGTCAGCAGTATGGTAGCTCACCTCGGACGTTCG
GCCAAGGGACCAAGGTGGAAATCAAACGA 3′
Amino acid sequence
(SEQ ID NO: 191)
5′EIVLTQSPGTLSLSPGERVTLSC RASQSVSSRYLA WYQQKPGQAPR
LLIY GASSRATGIPDRFSGSGSGTDFTLTISRLEPEDFAVYYC QQYGS
SPRT FGQGTKVEIKR 3′

TABLE 27
2.24.1 Light chain V region domains.

| | | AA | |
|---|---|---|---|
| REGION | SEQUENCE | RESIDUES* | SEQ ID |
| FRI | EIVLTQSPGTLSLSPGER | 1-23 | SEQ ID NO: 192 |
| | VTLSC | | |
| CDR1 | RASQSVSSRYLA | 24-35 | SEQ ID NO: 193 |
| FR2 | WYQQKPGQAPRLLIY | 36-50 | SEQ ID NO: 194 |
| CDR2 | GASSRAT | 51-57 | SEQ ID NO: 195 |
| FR3 | GIPDRFSGSGSGTDFTLTIS | 58-88 | SEQ ID NO: 196 |
| | RLEPEDFAVYY | | |
| CDR3 | QQYGSSPRT | 89-97 | SEQ ID NO: 197 |
| FR4 | FGQGTKVEIKR | 98-109 | SEQ ID NO: 198 |

*AA Residues of SEQ ID NO: 191

### Antibody-2.3.1

### Heavy Chain Variable Region

Nucleotide sequence
(SEQ ID NO: 199)
5′CAGGTGCAGCTGGTGCAGTCTGGGGCTGAGGTGAAGAAGCCTGGGGC
CTCAGTGAAGGTCTCCTGCAAGGCTTCTGGATACACCTTCACCGGCTAC
TATATGCACTGGGTGCGACAGGCCCCTGGACAAGGGCTTGAGTGGATGG
GATGGATCAACCCTAACAGTGGTGGCACAAACTATGCACAGAAGTTTCA
GGACAGGGTCACCATGACCAGGGACACGTCCATCAGCACAGCCTACATG
GAGCTGAGCAGGCTGAGATCTGACGACACGGCCGTGTATTACTGTGCGA
GAGATTTCTTTGGTTCGGGGAGTCTCCTCTACTTTGACTACTGGGGCCA
GGGAACCCTGGTCACCGTCTCCTCAGCC 3′
Amino acid sequence
(SEQ ID NO: 200)
5′QVQLVQSGAEVKKPGASVKVSCKASGYTFTGYYMHWVRQAPGQGLEWM
GWINPNSGGTNYAQKFQDRVTMTRDTSISTAYMELSRLRSDDTAVYYCAR
DFFGSGSLLYFDYWGQGTLVTVSSA 3′

TABLE 28
2.3.1 Heavy chain V region domains.

| | | AA | |
|---|---|---|---|
| REGION | SEQUENCE | RESIDUES* | SEQ ID |
| FRI | QVQLVQSGAEVKKPGASVKV | 1-25 | SEQ ID NO: 201 |
| | SCKAS | | |
| CDR1 | GYTFTGYYMH | 26-35 | SEQ ID NO: 202 |
| FR2 | WVRQAPGQGLEWMG | 36-49 | SEQ ID NO: 203 |
| CDR2 | WINPNSGGTNYAQKFQD | 50-66 | SEQ ID NO: 204 |
| FR3 | RVTMTRDTSISTAYMELSR | 67-98 | SEQ ID NO: 205 |
| | LRSDDTAVYYCAR | | |
| CDR3 | DFFGSGSLLYFDY | 99-111 | SEQ ID NO: 206 |
| FR4 | WGQGTLVTVSSA | 112-123 | SEQ ID NO: 207 |

*AA Residues of SEQ ID NO: 200

### Light Chain Variable Region

Nucleotide sequence
(SEQ ID NO: 208)
5′GATATTGTGATGACCCAGACTCCACTCTCTCTGTCCGTCACCCCTG
GACAGCCGGCCTCCATCTCCTGCAAGTCTAGTCAGAGCCTCCTGCATA
GTGGTGGAAAGACCTATTTGTATTGGTACCTGCAGAGGCCAGGCCAGC
CTCCACAGCTCCTGATCTATGAAGTTTCCAACCGGTTCTCTGGAGTGC
CAGATAGGTTCAGTGGCAGCGGGTCAGGGACAGATTTCACACTGAAAA
TCAGCCGGGTGGAGGCTGAGGATGTTGGGGTTTATTACTGCATGCAAA
GTATACACCTTCCGCTCACTTTCGGCGGAGGGACCAAGGTGGAGATCA
AACGA 3′
Amino acid sequence
(SEQ ID NO: 209)
5′DIVMTQTPLSLSVTPGQPASISC KSSQSLLHSGGKTYLY WYLQRP
GQPPQLLIY EVSNRFSGVPDRFSGSGSGTDFTLKISRVEAEDVGVYYC
MQSIHLPLT FGGGTKVEIKR 3′

TABLE 29
2.3.1 Light chain V region domains.

| | | AA | |
|---|---|---|---|
| REGION | SEQUENCE | RESIDUES* | SEQ ID |
| FRI | DIVMTQTPLSLSVTPGQPAS | 1-23 | SEQ ID NO: 210 |
| | ISC | | |
| CDR1 | KSSQSLLHSGGKTYLY | 24-39 | SEQ ID NO: 211 |
| FR2 | WYLQRPGQPPQLLIY | 40-54 | SEQ ID NO: 212 |
| CDR2 | EVSNRFS | 55-61 | SEQ ID NO: 213 |
| FR3 | GVPDRFSGSGSGTDFTLKIS | 62-93 | SEQ ID NO: 214 |
| | RVEAEDVGVYYC | | |
| CDR3 | MQSIHLPLT | 94-102 | SEQ ID NO: 215 |
| FR4 | FGGGTKVEIKR | 103-113 | SEQ ID NO: 216 |

*AA Residues of SEQ ID NO: 209

### Antibody-2.6.1

### Heavy Chain Variable Region

Nucleotide sequence
(SEQ ID NO: 309)
5′CAGGTGCAGCTGGTGCAGTCTGGGGCTGAGGTGAAGAAGCCTGGG
GCCTCAGTGAAGGTCTCCTGCAAGGCTTCTGGATACACCTTCACCGG
CTACTATATGCACTGGGTGCGACAGGCCCCTGGACAAGGGCTTGAGT
GGATGGGATGGATCAACCCTAACAGTGGTGGCACAAACTATGCACAG
AAGTTTCAGGACAGGGTCACCATGACCAGGGACACGTCCATCAGCAC
AGCCTACATGGAGCTGAGCAGGCTGAGATCTGACGACACGGCCGTGT
ATTACTGTGCGAGAGATTTCTTTGGTTCGGGGAGTCTCCTCTACTTT
GACTACTGGGGCCAGGGAACCCTGGTCACCGTCTCCTCAGCC 3′
Amino acid sequence
(SEQ ID NO: 310)
5′QVQLVQSGAEVKKPGASVKVSCKAS GYTFTGYYMH WVRQAPGQGLE
WMG WINPNSGGTNYAQKFQDRVTMTRDTSISTAYMELSRLRSDDTAVYY
CAR DFFGSGSLLYFDY WGQGTLVTVSSA 3′

TABLE 30
2.6.1 Heavy chain V region domains.

| | | AA | |
|---|---|---|---|
| REGION | SEQUENCE | RESIDUES* | SEQ ID |
| FRI | QVQLVQSGAEVKKPGASVKVSCKAS | 1-25 | 311 |
| CDR1 | GYTFTGYYMH | 26-35 | 312 |
| FR2 | WVRQAPGQGLEWMG | 36-49 | 313 |
| CDR2 | WINPNSGGTNYAQKFQD | 50-66 | 314 |
| FR3 | RVTMTRDTSISTAYMELSRLRSDDT | 67-98 | 315 |
| | AVYYCAR | | |
| CDR3 | DFFGSGSLLYFDY | 99-112 | 316 |
| FR4 | WGQGTLVTVSSA | 113-124 | 317 |

*AA Residues of SEQ ID NO: 310

### Light Chain Variable Region

Nucleotide sequence
(SEQ ID NO: 318)
5′GATATTGTGATGACCCAGACTCCACTCTCTCTGTCCGTCACCCCT
GGACAGCCGGCCTCCATCTCCTGCAAGTCTAGTCAGAGCCTCCTGCA
TAGTGGTGGAAAGACCTATTTGTATTGGTACCTGCAGAGGCCAGGCC
AGCCTCCACAGCTCCTGATCTATGAAGTTTCCAACCGGTTCTCTGGA
GTGCCAGATAGGTTCAGTGGCAGCGGGTCAGGGACAGATTTCACACT
GAAAATCAGCCGGGTGGAGGCTGAGGATGTTGGGGTTTATTACTGCA
TGCAAAGTATACACCTTCCGCTCACTTTCGGCGGAGGGACCAAGGTG
GAGATCAAACGA 3′
Amino acid sequence
(SEQ ID NO: 319)
5′DIVMTQTPLSLSVTPGQPASISC KSSQSLLHSGGKTYLY WYLQRP
GQPPQLLIY EVSNRFSGVPDRFSGSGSGTDFTLKISRVEAEDVGVYYC
MQSIHLPLT FGGGTKVEIKR 3′

TABLE 31
2.6.1 Light chain V region domains.

| | | AA | |
|---|---|---|---|
| REGION | SEQUENCE | RESIDUES* | SEQ ID |
| FRI | DIVMTQTPLSLSVTPGQPASISC | 1-23 | 320 |
| CDR1 | KSSQSLLHSGGKTYLY | 24-39 | 321 |
| FR2 | WYLQRPGQPPQLLIY | 40-54 | 322 |
| CDR2 | EVSNRFS | 55-61 | 323 |
| FR3 | GVPDRFSGSGSGTDFTLKISRVEA | 62-93 | 324 |
| | EDVGVYYC | | |
| CDR3 | MQSIHLPLT | 94-102 | 325 |
| FR4 | FGGGTKVEIKR | 103-113 | 326 |

*AA Residues of SEQ ID NO: 319

### Antibody-2.7.1

### Heavy Chain Variable Region

Nucleotide sequence
(SEQ ID NO: 217)
5′ CAGGTGCAGCTGGAGCAGTCGGGGGGAGGCGTGGTCCAGCCTGGGAG
GTCCCTGAGACTCTCCTGTGCAGCGTCTGGATTCACCTTCAATAACTATG
GCATGCACTGGGTCCGCCAGGCTCCAGGCAAGGGGCTGGAGTGGGTGGCA
GTTATATGGTATGATGGAAGTAATAAATACTATGCAGACTCCGTGAAGGG
CCGATTCACCATCTCCAGAGACAATTCCAAGAACACGCTGTATCTGCAAA
TGAACAGCCTGAGAGCCGAGGACACGGCTGTGTATTACTGTGCGAAAGAT
GAGGAATACTACTATGTTTCGGGGCTTGACTACTGGGGCCAGGGAACCCT
GGTCACCGTCTCCTCAGCC 3′
Amino acid sequence
(SEQ ID NO: 218)
5′ QVQLEQSGGGVVQPGRSLRLSCAAS GFTFNNYGMH
WVRQAPGKGLEWVA VIWYDGSNKYYADSVKG
RFTISRDNSKNTLYLQMNSLRAEDTAVYYCAK DEEYYYVSGLDY
WGQGTLVTVSSA 3′

TABLE 32
2.7.1 Heavy chain V region domains.

| | | AA | |
|---|---|---|---|
| REGION | SEQUENCE | RESIDUES* | SEQ ID |
| FRI | QVQLEQSGGGVVQPGRS | 1-25 | SEQ ID |
| | LRLSCAAS | | NO: 219 |
| CDR1 | GFTFNNYGMH | 26-35 | SEQ ID |
| | | | NO: 220 |
| FR2 | WVRQAPGKGLEWVA | 36-49 | SEQ ID |
| | | | NO: 221 |
| CDR2 | VIWYDGSNKYYADSVKG | 50-66 | SEQ ID |
| | | | NO: 222 |
| FR3 | RFTISRDNSKNTLYLQM | 67-98 | SEQ ID |
| | NSLRAEDTAVYYCAK | | NO: 223 |
| CDR3 | DEEYYYVSGLDY | 99-110 | SEQ ID |
| | | | NO: 224 |
| FR4 | WGQGTLVTVSSA | 111-122 | SEQ ID |
| | | | NO: 225 |

*AA Residues of SEQ ID NO: 218

### Light Chain Variable Region

Nucleotide sequence
(SEQ ID NO: 226)
5′ CTGACTCAGTCTCCATCCTCCCTGTCTGCATCTGTAAGAGACAGAGT
CACCATCACTTGCCGGGCGAGTCAGGACATTAGCAATTATTTAGCCTGGT
ATCAGCAGAAACCAGGGAAAGTTCCTAATCTCCTGATCTATGCTGCATCC
ACTTTGCAATCAGGGGTCCCATCTCGGTTCAGTGGCAGTGGATCTGGGAC
AGATTTCACTCTCACCATCAGCAGCCTGCAGCCTGAAGATGTTGCAACTT
ATTACTGTCAAAAGTATAACAGTGCCCCGCTCACTTTCGGCGGAGGGACC
AAGGTGGAGATCAAACGA 3′
Amino acid sequence
(SEQ ID NO: 227)
5′ LTQSPSSLSASVRDRVTITC RASQDISNYLA
WYQQKPGKVPNLLIY AASTLQS
GVPSRFSGSGSGTDFTLTISSLQPEDVATYYC QKYNSAPLT
FGGGTKVEIKR 3′

TABLE 33
2.7.1 Light chain V region domains.

| | | AA | |
|---|---|---|---|
| REGION | SEQUENCE | RESIDUES* | SEQ ID |
| FRI | LTQSPSSLSASVRDRVTITC | 1-20 | SEQ ID |
| | | | NO: 228 |
| CDR1 | RASQDISNYLA | 21-31 | SEQ ID |
| | | | NO: 229 |
| FR2 | WYQQKPGKVPNLLIY | 32-46 | SEQ ID |
| | | | NO: 230 |
| CDR2 | AASTLQ | 47-52 | SEQ ID |
| | | | NO: 231 |
| FR3 | GVPSRFSGSGSGTDFTLTIS | 53-84 | SEQ ID |
| | SLQPEDVATYYC | | NO: 232 |
| CDR3 | QKYNSAPLT | 85-93 | SEQ ID |
| | | | NO: 233 |
| FR4 | FGGGTKVEIKR | 94-104 | SEQ ID |
| | | | NO: 234 |

*AA Residues of SEQ ID NO: 227

### Antibody-2.8.1

### Heavy Chain Variable Region

Nucleotide sequence
(SEQ ID NO: 235)
5′ CAGATCACCTTGAAGGAGTCTGGTCCTACGCTGGTGACACCCACACA
GACCCTCACGCTGACCTGCACCTTCTCTGGGTTCTCACTCAGCACTGGTG
GAATGGGTGTGGGCTGGATCCGTCAGCCCCCAGGAAAGGCCCTGGACTGG
CTTACACTCATTTATTGGAATGATGATAAGCACTACAGCCCATCTCTGAA
GAGCAGGCTTACCATCACCAAGGACACCTCCAAAAACCAGGTGGTCCTTA
GAATGACCAACATGGACCCTGTGGACACAGCCACTTATTACTGTGCACAC
CTGCATTACGATATTTTGACTGGTTTTAACTTTGACTACTGGGGCCAGGG
AACCCTGGTCACCGTCTCCTCAGCC 3′
Amino acid sequence
(SEQ ID NO: 236)
5′ QITLKESGPTLVTPTQTLTLTCTFS GFSLSTGGMGVG
WIRQPPGKALDWLT LIYWNDDKHYSPSLKS
RLTITKDTSKNQVVLRMTNMDPVDTATYYCAH LHYDILTGFNFDY
WGQGTLVTVSSA 3′

TABLE 34
2.8.1 Heavy chain V region domains.

| | | AA | |
|---|---|---|---|
| REGION | SEQUENCE | RESIDUES* | SEQ ID |
| FRI | QITLKESGPTLVTPTQ | 1-25 | SEQ ID |
| | TLTLTCTFS | | NO: 237 |
| CDR1 | GFSLSTGGMGVG | 26-37 | SEQ ID |
| | | | NO: 238 |
| FR2 | WIRQPPGKALDWLT | 38-51 | SEQ ID |
| | | | NO: 239 |
| CDR2 | LIYWNDDKHYSPSLKS | 52-67 | SEQ ID |
| | | | NO: 240 |
| FR3 | RLTITKDTSKNQVVLR | 68-99 | SEQ ID |
| | MTNMDPVDTATYYCAH | | NO: 241 |
| CDR3 | LHYDILTGFNFDY | 100-112 | SEQ ID |
| | | | NO: 242 |
| FR4 | WGQGTLVTVSSA | 113-124 | SEQ ID |
| | | | NO: 243 |

*AA Residues of SEQ ID NO: 236

### Light Chain Variable Region

Nucleotide sequence
(SEQ ID NO: 244)
5′ GATATTGTGATGACCCAGACTCCACTCTCCCTGCCCGTCACCCCTGG
AGAGCCGGCCTCCATCTCCTGCAGGTCTAGTCAGAGCCTCTTGGATAGTG
ATGATGGAAACACCTATTTGGACTGGTACCTGCAGAAGCCAGGGCAGTCT
CCACAGCTCCTGATCTATACGCTTTCCTATCGGGCCTCTGGAGTCCCAGA
CAGGTTCAGTGGCAGTGGGTCAGGCACTGATTTCACACTGAAAATCAGCA
GGGTGGAGGCTGAGGATGTTGGAGTTTATTACTGCATGCAACGTATAGAG
TTTCCGCTCACTTTCGGCGGAGGGACCAAGGTGGAGATCAAACGA 3′
Amino acid sequence
(SEQ ID NO: 245)
5′ DIVMTQTPLSLPVTPGEPASISC RSSQSLLDSDDGNTYLD
WYLQKPGQSPQLLIY TLSYRAS
GVPDRFSGSGSGTDFTLKISRVEAEDVGVYYC MQRIEFPLT
FGGGTKVEIKR 3′

TABLE 35
2.8.1 Light chain V region domains.

| | | AA | |
|---|---|---|---|
| REGION | SEQUENCE | RESIDUES* | SEQ ID |
| FRI | DIVMTQTPLSLPVTPGEPASISC | 1-23 | SEQ ID |
| | | | NO: 246 |
| CDR1 | RSSQSLLDSDDGNTYLD | 24-40 | SEQ ID |
| | | | NO: 247 |
| FR2 | WYLQKPGQSPQLLIY | 41-55 | SEQ ID |
| | | | NO: 248 |
| CDR2 | TLSYRAS | 56-62 | SEQ ID |
| | | | NO: 249 |
| FR3 | GVPDRFSGSGSGTDFTLKISRVE | 63-94 | SEQ ID |
| | AEDVGVYYC | | NO: 250 |
| CDR3 | MQRIEFPLT | 95-103 | SEQ ID |
| | | | NO: 251 |
| FR4 | FGGGTKVEIKR | 103-114 | SEQ ID |
| | | | NO: 252 |

*AA Residues of SEQ ID NO: 245

### VH4-31 Derived Anti-GPNMB Antibodies:

In a particular embodiment, GPNMB-binding human antibodies of the invention comprise germline V heavy chain region VH4-31 or are derived therefrom and have an amino acid sequence of the formula:

X₁SGPGLVKPSQX₂LSLTCTVS GGSIS SX₃X₄YX₅WX₆
WIRX₇HPGKGLEWIG YIYYSGX₈TYX₉NPSLKS
RVX₁₀ISVDTSKNQFSLX₁₁LSSVTAADTAVYYCAR

Where: X1 is E or Q X2 is T or N; X3 is A, F or G; X4 is N or G; X5 is Y or F; X6 is T or S; X7 is Q or H; X8 is S or N; X9 is C, S or Y; X10 is I or T; X11 is K or T; (SEQ ID NO:253).

In specific embodiments SEQ ID NO:253 is combined with D3-22 or D1-20. Furthermore the combination of SEQ ID NO:253 with D3-22 or D1-20 is combined with JH6b or JH4b and in specific embodiments, after affinity maturation these GPNMB-binding human antibodies, for example Mab1.15.1, Mab1.7.1 and Mab2.22.1, have amino acid sequences SEQ ID NOs:20, 56 and 164 and can be encoded by nucleotide sequences SEQ ID NO:19, 55 and 163.

Furthermore, in particular embodiments H chain CDR1 sequences are the germline VH4-31 CDR or affinity matured sequences thereof, of the formula:

CDR1: GGSIS SX₃X₄YX₅WX₆

Where: X3 is A, F or G; X4 is N or G; X5 is Y or F; X6 is T or S; (SEQ ID NO:254).

In specific embodiments an anti-GPNMB antibody of the invention comprise a CDR1 sequence selected from the following: SEQ ID NO:22, 58, 166.

In particular embodiments H chain CDR2 sequences are the germline VH4-31 CDR or affinity matured sequences thereof of the formula:

CDR2: YIYYSGX₈TYX₉NPSLKS

Where: X8 is S or N; X9 is C, S or Y; (SEQ ID NO:255).

In specific embodiments an anti-GPNMB antibody of the invention comprise a CDR2 sequence selected from the following: SEQ ID NO: 24, 60, and 168.

In particular embodiments, the H chain CDR3 sequence is a D3-22, JH6b combination having SEQ ID NO:170. Alternatively, in particular embodiments the H chain CDR3 sequence is a D1-20, JH4b combination having SEQ ID NO:26 or 62.

### VH1-2 Derived Anti-GPNMB Antibodies:

In a particular embodiment, GPNMB-binding human antibodies of the invention comprise germline V heavy chain region VH1-2 or are derived therefrom and include an amino acid sequence of the formula:

QLVQSGAEVKKPGASVKVSCKAS GYTFT GX₁YMH
WVRQX₂PGQGLEWMG WINPNSGGTX₃YX₄QKFQX₅
RVTMTRDTSISTX₆YMELSRLRSDDTAVYYCAR

Where: X1 is Y or F; X2 is A or T; X3 is N or Y; X4 is A or V; X5 is D or G; X6 is A or V; (SEQ ID NO: 256).

In specific embodiments SEQ ID NO:256 is combined with D3-10 or D6-19. Furthermore the combination ov SEQ ID NO:256 with D3-10 or D6-19 is combined with JH4b or JH5b and in specific embodiments, after affinity maturation these GPNMB-binding human antibodies, for example Mab2.3.1 and Mab 2.17.1 have amino acid sequences: SEQ ID NO:128 and 200 and can be encoded by nucleotide sequences SEQ ID NO:127 and 199.

Furthermore, in particular embodiments H chain CDR1 sequences are the germline VH1-2 CDR or affinity matured sequences thereof, of the formula:

CDR1: GYTFTGX₁YMH Where: X1 is Y or F, (SEQ ID NO:257)

In specific embodiments an anti-GPNMB antibody of the invention comprise a CDR1 sequence selected from SEQ ID NO: 130 and 202.

In particular embodiments H chain CDR2 sequences are the germline VH1-2 CDR or affinity matured sequences thereof of the formula:

CDR2: WINPNSGGTX₃YX₄QKFQX₅

Where: X3 is N or Y; X4 is A or V; X5 is D or G (SEQ ID NO:258).

In specific embodiments an anti-GPNMB antibody of the invention comprise a CDR2 sequence selected from SEQ ID NO:132 and 204.

In particular embodiments H chain CDR3 sequences are germline D3-10, JH4b combinations or affinity matured sequences thereof, having the amino acid sequence of the formula:

CDR3: X₁X₂X₃GSGSX₄X₅

Where: X1 is Y or D; X2 is Y or F; X3 is Y or F; X4 is Y or L; X5 is Y or L (SEQ ID NO:259).

In specific embodiments an anti-GPNMB antibody of the invention comprise a CDR3 sequence selected from SEQ ID NO:134 and 206.

### VH2-5 Derived Anti-GPNMB Antibodies:

In a particular embodiment, GPNMB-binding human antibodies of the invention comprise germline V heavy chain region VH2-5 or are derived therefrom and include an amino acid sequence of the formula:

ITLKESGPTLVX₁PTQTLTLTCTFSGFSLSX₂X₃GX₄GVGWIRQPPGKAL
X₅WLX₆LIYWNDDKX₇YSPSLX₈SRLTITKDTSKNQVVLX₉X₁₀TNMDP
VDTATYYCAH

Where: X1 is K or T; X2 is T or A; X3 is S or G; X4 is M or V; X5 is D or E; X6 is A or T; X7 is R or H; X8 is K or R; X9 is T or R; X10 is M or I; (SEQ ID NO:260).

In specific embodiments SEQ ID NO:260 is combined with D3-9 or D3-16 and furthermore is combined with JH4b. In specific embodiments, after affinity maturation these GPNMB-binding human antibodies, for example, Mab 2.8.1 and Mab 1.2.2 have amino acid sequences SEQ ID NO: 38 and 236 and can be encoded by nucleotide sequences SEQ ID NO: 37 and 235.

Furthermore, in particular embodiments H chain CDR1 sequences are the germline VH2-5 CDR or affinity matured sequences thereof, of the formula:

CDR1:
GFSLS X₂X₃GX₄GVG

Where: X2 is T or A; X3 is S or G; X4 is M or V; (SEQ ID NO:261).

In specific embodiments an anti-GPNMB antibody of the invention comprise a CDR1 sequence selected from SEQ ID NO: 40 and 238.

In particular embodiments H chain CDR2 sequences are the germline VH2-5 CDR2 or affinity matured sequences thereof of the formula:

CDR2:
LIYWNDDKX₇YSPSLX₈S

Where: X7 is R or H; X8 is K or R; (SEQ ID NO:262).

In specific embodiments an anti-GPNMB antibody of the invention comprise a CDR2 sequence selected from SEQ ID NO:42 and 240.

In particular embodiments H chain CDR3 sequences are germline D3-9, JH4b combinations or affinity matured sequences thereof and include an amino acid sequence of the formula:

CDR3:
X₁YDILTGX₂X₃

Where: X1 is Y or H; X2 is Y or F; and X3 is Y or N (SEQ ID NO:263).

In a specific embodiments an anti-GPNMB antibody of the invention comprises a CDR3 amino acid sequence SEQ ID NO:242.

In yet another particular embodiment H chain CDR3 sequences are germline D3-16, JH4b combinations or affinity matured sequences thereof and include an amino acid sequence of the formula:

CDR3:
YDYX₁WGS

Where: X1 is V or D (SEQ ID NO:264).

In a specific embodiment an anti-GPNMB antibody of the invention comprises a CDR3 amino acid sequence SEQ ID NO: 44.

### VH3-33 Derived Anti-GPNMB Antibodies:

In a particular embodiment, GPNMB-binding human antibodies of the invention comprise germline V heavy chain region VH3-33 or are derived therefrom and have an amino acid sequence of the formula:

QVQLX₁X₂SGGGVVQPGRSLRLSCAASGFTFX₃X₄YGX₅HW
VRQAPGKGLEWVAVIWX₆DGX₇NKYYADSVKGRFTISRDN
SKNTLYLQMNSLRAEDX₈AVYYCAX₉

Where: X1 is V or E; X2 is E or Q; X3 is S or N; X4 is S or N; X5 is M or I; X6 is Y or F; X7 is S or R; X8 is T or A; X9 is R or K (SEQ ID NO:265).

In specific embodiments SEQ ID NO:265 is combined with D3-10 or D4-17 and furthermore with JH4b. In specific embodiments, after affinity maturation these GPNMB-binding human antibodies, for example Mab 2.7.1 and Mab2.15.1 have amino acid sequences: SEQ ID NO:92 and 218 and can be encoded by nucleotide sequences SEQ ID NO:91 and 217.

Furthermore, in particular embodiments H chain CDR1 sequences are the germline VH3-33 CDR or affinity matured sequences thereof, of the formula:

CDR1:
GFTFX₃X₄YGX₅H

Where: X3 is S or N; X4 is S or N; X5 is M or I; (SEQ ID NO:266).

In specific embodiments an anti-GPNMB antibody of the invention comprise a CDR1 amino acid sequence selected from SEQ ID NO:94 and 220.

In particular embodiments H chain CDR2 sequences are the germline VH3-33 CDR2 or affinity matured sequences thereof of the formula:

CDR2:
VIWX₆DGX₇NKYYADSVKG

Where: X6 is Y or F; X7 is S or R; (SEQ ID NO:267).

In specific embodiments an anti-GPNMB antibody of the invention comprise a CDR2 sequence selected from SEQ ID NO:96 and 222.

In particular embodiments H chain CDR3 sequences are D3-10, JH4b combinations or affinity matured sequences thereof and include an amino acid sequence of the formula:

CDR3:
YYYGSGX₁

Where: X1 is S or L (SEQ ID NO:268).

A specific embodiment is anti-GPNMB antibody 2.7.1 having a CDR3 amino acid sequence SEQ ID NO:224.

In an alternative embodiment H chain CDR3 sequences are D4-17, JH4b combinations or affinity matured sequences thereof and include an amino acid sequence of the formula:

CDR3:
DYGDX₁

Where: X1 is Y or S (SEQ ID NO:269).

A specific embodiment is anti-GPNMB antibody 2.15.1 having a CDR3 amino acid sequence SEQ ID NO: 98.

### VH3-11 Derived Anti-GPNMB Antibodies:

In a particular embodiment, GPNMB-binding human antibodies of the invention comprise germline V heavy chain region VH3-11 or are derived therefrom and have an amino acid sequence of the formula:

QVQLVESGGGLVKPGGSLRLSCAASGFTFSX₁YX₂MX₃
WIRQAPGKGLEWVSYISX₄SGSX₅X₆X₇YADSVKGRFT
X₈SRDNAKNSLYLQMNSLRAEDTAVYYCAR

Where: X1 is D or S; X2 is S or Y; X3 is S or T; X4 is S or I; X5 is T or I; X6 is T or I; X7 is Y or H; X8 is I or M; (SEQ ID NO:270).

In specific embodiments SEQ ID NO:270 is combined with D6-13 and furthermore with JH3b. In specific embodiments, after affinity maturation these GPNMB-binding human antibodies, for example Mab 2.16.1 have amino acid sequence SEQ ID NO:110 and can be encoded by nucleotide sequence SEQ ID NO:109.

Furthermore, in particular embodiments H chain CDR1 sequences are the germline VH3-11 CDR1 or affinity matured sequences thereof, of the formula:

CDR1:
GFTFS X₁YX₂MX₃

Where: X1 is D or S; X2 is S or Y; X3 is S or T; (SEQ ID NO:271).

In specific embodiments an anti-GPNMB antibody of the invention comprise a CDR1 amino acid sequence SEQ ID NO:112.

In particular embodiments H chain CDR2 sequences are the germline VH3-11 CDR2 or affinity matured sequences thereof of the formula:

CDR2:
YISX₄SGSX₅X₆X₇YADSVKG

Where: X4 is S or I; X5 is T or I; X6 is T or I; X7 is Y or H; (SEQ ID NO:272).

In specific embodiments an anti-GPNMB antibody of the invention comprises a CDR2 sequence SEQ ID NO:114.

In particular embodiments H chain CDR3 sequences are D6-13, JH3b combinations or affinity matured sequences thereof and include an amino acid sequence of the formula:

CDR3:
X₁X₂AAAG--AFDI

Where: X1 is G or D; X2 is I or G; (SEQ ID NO:273).

A specific embodiment is anti-GPNMB antibody 2.16.1 having a CDR3 amino acid sequence SEQ ID NO:116.

### VH3-21 Derived Anti-GPNMB Antibodies:

In a particular embodiment, GPNMB-binding human antibodies of the invention comprise germline V heavy chain region VH3-21 or are derived therefrom and have an amino acid sequence of the formula:

X₁VQLX₂X₃SGGGLVKPGGSLRX₄SCAASGFTFSSYSM
NWVRQAPGKGLEWVS X₅ISS SSSYIYYADSVKG
RFTISRDNAKNSLYLQMNSLRAEDTAVYYCAR

Where: X1 is E or Q; X2 is V or E; X3 is E or Q; X4 is F or L; X5 is S or F; (SEQ ID NO:274).

In specific embodiments SEQ ID NO:274 is combined with D1-26 and furthermore with JH4b. In specific embodiments, after affinity maturation these GPNMB-binding human antibodies, for example Mab 2.21.1 have amino acid sequence SEQ ID NO:146 and can be encoded by nucleotide sequence SEQ ID NO:145.

Furthermore, in particular embodiments H chain CDR1 sequences are the germline VH3-21 CDR1, SEQ ID NO:148 or affinity matured sequences thereof.

In particular embodiments H chain CDR2 sequences are the germline VH3-21 CDR2 or affinity matured sequences thereof of the formula:

CDR2:
X₅ISS SSSYIYYADSVKG

Where: X5 is S or F; (SEQ ID NO:275).

In specific embodiments an anti-GPNMB antibody of the invention comprises a CDR2 amino acid sequence SEQ ID NO:150.

In particular embodiments H chain CDR3 sequences are D1-26, JH4b combinations or affinity matured sequences thereof and include an amino acid sequence of the formula:

CDR3: X₁X₂VGAT-FDY

Where: X1 is G or D; X2 is I or W; (SEQ ID NO:276).

A specific embodiment is anti-GPNMB antibody 2.21.1 having a CDR3 amino acid sequence SEQ ID NO:152.

### VH3-30 Derived Anti-GPNMB Antibodies:

In a particular embodiment, GPNMB-binding human antibodies of the invention comprise germline V heavy chain region VH3-30 or are derived therefrom and include an amino acid sequence of the formula:

QLVESGGGVVQPGRSLRLSCAAS GFX₁FS SYGMH WVRQAPG
KGLEWVA VISYDGX₂NKYYADSVKG RFTISRDNSKNTLYLQM
NSLRAEDTAVYYCAK

Where: X1 is T or A; X2 is S or N; (SEQ ID NO:277).

In specific embodiments SEQ ID NO:277 is combined with D3-10 and furthermore with JH6b. In specific embodiments, after affinity maturation these GPNMB-binding human antibodies, for example Mab 2.10.2 have amino acid sequence SEQ ID NO:74 and can be encoded by nucleotide sequence SEQ ID NO:73.

Furthermore, in particular embodiments H chain CDR1 sequences are the germline VH3-30 CDR1, or affinity matured sequences thereof having an amino acid sequence of the formula:

GFX₁FS SYGMH

Where: X1 is T or A; (SEQ ID NO:278).

In specific embodiments an anti-GPNMB antibody of the invention comprise a CDR1 sequence SEQ ID NO:76.

In particular embodiments H chain CDR2 sequences are the germline VH3-30 CDR2 or affinity matured sequences thereof of the formula:

CDR2: VISYDGX₂NKYYADSVKG

Where: X2 is S or N; (SEQ ID NO:279).

In specific embodiments an anti-GPNMB antibody of the invention comprises a CDR2 amino acid sequence SEQ ID NO:78.

In particular embodiments H chain CDR3 sequences are D3-10, JH6b combinations or affinity matured sequences thereof and include an amino acid sequence of the formula:

CDR3: X₁X₂X₃VRGX₄X₅X₆

Where: X1 is I or D; X2 is T or L; X3 is M or V; X4 is V or I; X5 is I or R; X6 is I or G; (SEQ ID NO:280).

A specific embodiment is anti-GPNMB antibody 2.10.2 having a CDR3 amino acid sequence SEQ ID NO:80.

### VH4-59 Derived Anti-GPNMB Antibodies:

In a particular embodiment, GPNMB-binding human antibodies of the invention comprise germline V heavy chain region VH4-59 or are derived therefrom and include an amino acid sequence of the formula:

QVQLQESGPGLVKPSETLSLTCTVS GX₁SIS X₂YYWS
WIRQPPGKGLEWIG YX₃YYSGSTNYNPSLKS RVTISV
DTSKNQFSLKLSSVTAADTAVYYCAR

Where: X1 is G or D; X2 is S or N; X3 is I or F; (SEQ ID NO:281).

In specific embodiments SEQ ID NO:281 is combined with D6-19 and furthermore with JH4b. In specific embodiments, after affinity maturation these GPNMB-binding human antibodies, for example Mab 1.10.2 have amino acid sequence SEQ ID NO:2 and can be encoded by nucleotide sequence SEQ ID NO:1.

Furthermore, in particular embodiments H chain CDR1 sequences are the germline VH4-59 CDR1, or affinity matured sequences thereof having an amino acid sequence of the formula:

GX₁SIS X₂YYWS

Where: X1 is G or D; X2 is S or N; (SEQ ID NO:282).

In specific embodiments an anti-GPNMB antibody of the invention comprise a CDR1 sequence SEQ ID NO:4.

In particular embodiments H chain CDR2 sequences are the germline VH4-59 CDR2 or affinity matured sequences thereof of the formula:

CDR2: YX₃YYSGSTNYNPSLKS

Where: X3 is I or F; (SEQ ID NO:283).

In specific embodiments an anti-GPNMB antibody of the invention comprises a CDR2 amino acid sequence SEQ ID NO:6.

In particular embodiments H chain CDR3 sequences are D6-19, JH4b combinations or affinity matured sequences thereof and include an amino acid sequence of the formula:

CDR3: X₁X₂GW---DY

Where: X1 is S or D; X2 is S or R; (SEQ ID NO:284).

A specific embodiment is anti-GPNMB antibody 1.10.2 having a CDR3 amino acid sequence SEQ ID NO:8.

### VH5-51 Derived Anti-GPNMB Antibodies:

In a particular embodiment, GPNMB-binding human antibodies of the invention comprise germline V heavy chain region VH5-51 or are derived therefrom and include an amino acid sequence of the formula:

QLVQSGAEVKKPGESLKISCX₁GS GYX₂FT X₃YWIGWV
RQMPGKGLEWMG X₄IYPX₅DSDTRYSPSFQG QVTISAD
KSISTAYLQWSSLKASDTAX₆YYCAR

Where: X1 is K or Q; X2 is S or I; X3 is S or N; X4 is I or V; X5 is G or D; X6 is M or I; (SEQ ID NO:285).

In specific embodiments SEQ ID NO:285 is combined with D5-24 and furthermore with JH4b. In specific embodiments, after affinity maturation these GPNMB-binding human antibodies, for example Mab 2.24.1 have amino acid sequence SEQ ID NO:182 and can be encoded by nucleotide sequence SEQ ID NO:181.

Furthermore, in particular embodiments H chain CDR1 sequences are the germline VH5-51 CDR1, or affinity matured sequences thereof having an amino acid sequence of the formula:

GYX₂FT X₃YWIG

Where: X2 is S or I; X3 is S or N; (SEQ ID NO:286).

In specific embodiments an anti-GPNMB antibody of the invention comprise a CDR1 sequence SEQ ID NO:184.

In particular embodiments H chain CDR2 sequences are the germline VH5-51 CDR2 or affinity matured sequences thereof of the formula:

CDR2: X₄IYPX₅DSDTRYSPSFQG

Where: X4 is I or V; X5 is G or D; (SEQ ID NO:287).

In specific embodiments an anti-GPNMB antibody of the invention comprises a CDR2 amino acid sequence SEQ ID NO:186.

In particular embodiments H chain CDR3 sequences are D5-24, JH4b combinations or affinity matured sequences thereof and include an amino acid sequence of the formula:

CDR3: X₁WLQX2--FDY

Where: X1 is R or K; X2 is L or H; (SEQ ID NO:288).

A specific embodiment is anti-GPNMB antibody 2.24.1 having a CDR3 amino acid sequence SEQ ID NO:188.

The antibodies of the invention bind an epitope of GPNMB (SEQ ID NO:289), preferably within the mature sequence of GPNMB and more preferably within the extracellular domain (ECD) of GPNMB.

Antibodies of the invention bind GPNMB with an affinity of 10⁻⁶ to 10⁻¹¹. Preferably with an affinity of 10⁻⁷ or greater and even more preferably 10⁻⁸ or greater. In a preferred embodiment, antibodies described herein bind to GPNMB with very high affinities (Kd), for example a human antibody that is capable of binding GPNMB with a Kd less than but not limited to 10⁻⁷, 10⁻⁹, 10⁻¹⁰, 10⁻¹¹, 10⁻¹², 10⁻¹³ or 10⁻¹⁴ M, or any range or value therein. Affinity and/or avidity measurements can be measured by KinExA® and/or BIACORE®, as described herein. In particular embodiments antibodies of the invention bind to GPNMB with Kds ranging from 50 to 150 pM.

Epitope mapping and secondary and tertiary structure analyses can be carried out to identify specific 3D structures assumed by the disclosed antibodies and their complexes with antigens (see, e.g., Epitope Mapping Protocols, ed. Morris, Humana Press, 1996). Such methods include, but are not limited to, X-ray crystallography (Biochem. Exp. Biol., 11:7-13, 1974) and computer modeling of virtual representations of the presently disclosed antibodies (Fletterick et al. (1986) Computer Graphics and Molecular Modeling, in Current Communications in Molecular Biology, Cold Spring Harbor Laboratory, Cold Spring Harbor, N.Y.).

Furthermore, the specific part of the protein immunogen recognized by antibody may be determined by assaying the antibody reactivity to parts of the protein, for example an N terminal and C terminal half. The resulting reactive fragment can then be further dissected, assaying consecutively smaller parts of the immunogen with the antibody until the minimal reactive peptide is defined. Alternatively, the binding specificity, that is the epitope, of anti-GPNMB antibodies of the invention may be determined by subjecting GPNMB immunogen to SDS-PAGE either in the absence or presence of a reduction agent and analyzed by immunoblotting. Epitope mapping may also be performed using SELDI. SELDI ProteinChip® (LumiCyte) arrays used to define sites of protein-protein interaction. GPNMB protein antigen or fragments thereof may be specifically captured by antibodies covalently immobilized onto the PROTEINCHIP array surface. The bound antigens may be detected by a laser-induced desorption process and analyzed directly to determine their mass.

The epitope recognized by anti-GPNMB antibodies described herein may be determined by exposing the PROTEINCHIP Array to a combinatorial library of random peptide 12-mer displayed on Filamentous phage (New England Biolabs). Antibody-bound phage are eluted and then amplified and taken through additional binding and amplification cycles to enrich the pool in favor of binding sequences. After three or four rounds, individual binding clones are further tested for binding by phage ELISA assays performed on antibody-coated wells and characterized by specific DNA sequencing of positive clones.

### Derivatives

This disclosure also provides a method for obtaining an antibody specific for GPNMB. CDRs in such antibodies are not limited to the specific sequences of H and L variable domains identified in Table 1 and may include variants of these sequences that retain the ability to specifically bind GPNMB. Such variants may be derived from the sequences listed in Table 1 by a skilled artisan using techniques well known in the art. For example, amino acid substitutions, deletions, or additions, can be made in the FRs and/or in the CDRs. While changes in the FRs are usually designed to improve stability and immunogenicity of the antibody, changes in the CDRs are typically designed to increase affinity of the antibody for its target. Variants of FRs also include naturally occurring immunoglobulin allotypes. Such affinity-increasing changes may be determined empirically by routine techniques that involve altering the CDR and testing the affinity of the antibody for its target. For example, conservative amino acid substitutions can be made within any one of the disclosed CDRs. Various alterations can be made according to the methods described in the art (Antibody Engineering, 2.sup.nd ed., Oxford University Press, ed. Borrebaeck, 1995). These include but are not limited to nucleotide sequences that are altered by the substitution of different codons that encode a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Substitutes for an amino acid within the sequence may be selected from other members of the class to which the amino acid belongs (see Table 3). Furthermore, any native residue in the polypeptide may also be substituted with alanine (Acta Physiol. Scand. Suppl. 643:55-67, 1998; Adv. Biophys. 35:1-24, 1998).

TABLE 3
Amino acid substitutions

| Original aa | | Preferred |
|---|---|---|
| Residue | Possible Substitutions | substitution |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, 1,4-Diamino-butyric Acid, Gln, Asn | Arg |
| Met (M) | Leu, Phe, Ile | Leu |
| Phe (F) | Leu, Val, Ile, Ala, Tyr | Leu |
| Pro (P) | Ala Gly | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, Norleucine | Leu |

Derivatives and analogs of antibodies of the invention can be produced by various techniques well known in the art, including recombinant and synthetic methods (Maniatis (1990) Molecular Cloning, A Laboratory Manual, 2.sup.nd ed., Cold Spring Harbor Laboratory, Cold Spring Harbor, N.Y., and Bodansky et al. (1995) The Practice of Peptide Synthesis, 2.sup.nd ed., Spring Verlag, Berlin, Germany).

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in the art (for example, Proteins, Structures and Molecular Principles (Creighton, Ed., W. H. Freeman and Company, New York (1984)).

In one embodiment, a method for making an H variable domain which is an amino acid sequence variant of an H variable domain of the invention comprises a step of adding, deleting, substituting, or inserting one or more amino acids in the amino acid sequence of the presently disclosed H variable domain, optionally combining the H variable domain thus provided with one or more L variable domains, and testing the H variable domain or H variable/L variable combination or combinations for specific binding to GPNMB or and, optionally, testing the ability of such antigen-binding domain to modulate GPNMB activity. The L variable domain may have an amino acid sequence that is identical or is substantially as set out according to Table 1.

An analogous method can be employed in which one or more sequence variants of a L variable domain disclosed herein are combined with one or more H variable domains.

A further aspect of the disclosure provides a method of preparing antigen-binding fragment that specifically binds with GPNMB. The method comprises: (a) providing a starting repertoire of nucleic acids encoding a H variable domain that either includes a CDR3 to be replaced or lacks a CDR3 encoding region; (b) combining the repertoire with a donor nucleic acid encoding an amino acid sequence substantially as set out herein for a H variable CDR3 such that the donor nucleic acid is inserted into the CDR3 region in the repertoire, so as to provide a product repertoire of nucleic acids encoding a H variable domain; (c) expressing the nucleic acids of the product repertoire; (d) selecting a binding fragment specific for GPNMB; and (e) recovering the specific binding fragment or nucleic acid encoding it.

Again, an analogous method may be employed in which a L variable CDR3 of the invention is combined with a repertoire of nucleic acids encoding a L variable domain, which either include a CDR3 to be replaced or lack a CDR3 encoding region. The donor nucleic acid may be selected from nucleic acids encoding an amino acid sequence substantially as set out in SEQ ID NOs: 2, 20, 38, 56, 74, 92, 110, 128, 146, 164, 182, 200, 218, 236, 253, 256, 260, 265, 270, 274, 277, 281, 285, 11, 29, 47, 65, 83, 101, 119, 137, 155, 173, 191, 209, 227 and 245. A sequence encoding a CDR of the invention (e.g., CDR3) may be introduced into a repertoire of variable domains lacking the respective CDR (e.g., CDR3), using recombinant DNA technology, for example, using methodology described by Marks et al. (Bio/Technology (1992) 10: 779-783). In particular, consensus primers directed at or adjacent to the 5′ end of the variable domain area can be used in conjunction with consensus primers to the third framework region of human H variable genes to provide a repertoire of H variable domains lacking a CDR3. The repertoire may be combined with a CDR3 of a particular antibody. Using analogous techniques, the CDR3-derived sequences may be shuffled with repertoires of H variable or L variable domains lacking a CDR3, and the shuffled complete H variable or L variable domains combined with a cognate L variable or H variable domain to make the GPNMB specific antibodies of the invention. The repertoire may then be displayed in a suitable host system such as the phage display system such as described in WO92/01047 so that suitable antigen-binding fragments can be selected.

Analogous shuffling or combinatorial techniques may be used (e.g. Stemmer, Nature (1994) 370: 389-391). In further embodiments, one may generate novel H variable or L variable regions carrying one or more sequences derived from the sequences disclosed herein using random mutagenesis of one or more selected H variable and/or L variable genes, such as error-prone PCR (Proc. Nat. Acad. Sci. U.S.A. (1992) 89: 3576-3580). Another method that may be used is to direct mutagenesis to CDRs of H variable or L variable genes (Proc. Nat. Acad. Sci. U.S.A. (1994) 91: 3809-3813; J. Mol. Biol. (1996) 263: 551-567). Similarly, one or more, or all three CDRs may be grafted into a repertoire of H variable or L variable domains, which are then screened for an antigen-binding fragment specific for GPNMB.

A portion of an immunoglobulin variable domain will comprise at least one of the CDRs substantially as set out herein and, optionally, intervening framework regions as set out herein. The portion may include at least about 50% of either or both of FR1 and FR4, the 50% being the C-terminal 50% of FR1 and the N-terminal 50% of FR4. Additional residues at the N-terminal or C-terminal end of the substantial part of the variable domain may be those not normally associated with naturally occurring variable domain regions. For example, construction of antibodies by recombinant DNA techniques may result in the introduction of N- or C-terminal residues encoded by linkers introduced to facilitate cloning or other manipulation steps. Other manipulation steps include the introduction of linkers to join variable domains to further protein sequences including immunoglobulin heavy chain constant regions, other variable domains (for example, in the production of diabodies), or proteinaceous labels as discussed in further detail below.

Although the embodiments illustrated in the Examples comprise a “matching” pair of H variable and L variable domains, a skilled artisan will recognize that alternative embodiments may comprise antigen-binding fragments containing only a single CDR from either L variable or H variable domain. Either one of the single chain specific binding domains can be used to screen for complementary domains capable of forming a two-domain specific antigen-binding fragment capable of, for example, binding to GPNMB. The screening may be accomplished by phage display screening methods using the so-called hierarchical dual combinatorial approach disclosed in WO92/01047, in which an individual colony containing either an H or L chain clone is used to infect a complete library of clones encoding the other chain (L or H) and the resulting two-chain specific binding domain is selected in accordance with phage display techniques as described.

Anti-GPNMB antibodies described herein can be linked to another functional molecule, e.g., another peptide or protein (albumin, another antibody, etc.), toxin, radioisotope, cytotoxic or cytostatic agents. For example, the antibodies can be linked by chemical cross-linking or by recombinant methods. The antibodies may also be linked to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337. The antibodies can be chemically modified by covalent conjugation to a polymer, for example, to increase their circulating half-life. Exemplary polymers and methods to attach them are also shown in U.S. Pat. Nos. 4,766,106; 4,179,337; 4,495,285, and 4,609,546.

The disclosed antibodies may also be altered to have a glycosylation pattern that differs from the native pattern. For example, one or more carbohydrate moieties can be deleted and/or one or more glycosylation sites added to the original antibody. Addition of glycosylation sites to the presently disclosed antibodies may be accomplished by altering the amino acid sequence to contain glycosylation site consensus sequences known in the art. Another means of increasing the number of carbohydrate moieties on the antibodies is by chemical or enzymatic coupling of glycosides to the amino acid residues of the antibody (WO 87/05330; CRC Crit. Rev. Biochem., 22: 259-306, 1981). Removal of any carbohydrate moieties from the antibodies may be accomplished chemically or enzymatically (Arch. Biochem. Biophys., 259: 52, 1987; Anal. Biochem., 118: 131, 1981; Meth. Enzymol., 138: 350, 1987). The antibodies may also be tagged with a detectable, or functional, label. Detectable labels include radiolabels such as ¹³¹I or ⁹⁹Tc, which may also be attached to antibodies using conventional chemistry. Detectable labels also include enzyme labels such as horseradish peroxidase or alkaline phosphatase. Detectable labels further include chemical moieties such as biotin, which may be detected via binding to a specific cognate detectable moiety, e.g., labeled avidin.

The valency of the antibodies may be custom designed to affect affinity and avidity, retention time at binding sites (see e.g. Am H. Pathol, 2002 160:1597-1608; J. Med. Chem. 2002 45:2250-2259; Br. J. Cancer 2002 86:1401-1410; Biomol. Eng. 2001 18:95-108; Int J. Cancer 2002 100:367-374).

Multiple specificity (bifunctional) binding reagents may be designed based upon the GPNMB specific sequences of the invention (Biomol. Eng. 2001 18:31-40). For example, a bispecific or bifunctional antibody is an artificial hybrid antibody having two different heavy/light chain pairs and two different binding sites. Bispecific antibodies can be produced by a variety of methods including fusion of hybridomas or linking of Fab′ fragments (Clin. Exp. Immunol. 1990, 79: 315-321; J. Immunol. 199, 2148:1547-1553). Such bispecific antibodies can be generated comprising a specificity to GPNMB and a second specificity to a second molecule using techniques that are well known (Immunol Methods 1994, 4:72-81; Wright and Harris, supra.; Traunecker et al.1992 Int. J. Cancer (Suppl.) 7:51-52). Bispecific antibodies prepared in this manner selectively kill cells expressing GPNMB.

Antibodies, in which CDR sequences differ only insubstantially from those set out in SEQ ID NOs: 4, 6, 8, 13, 15, 17, 22, 24, 26, 31, 33, 35, 40, 42, 44, 49, 51, 53, 58, 60, 62, 67, 69, 71, 76, 78, 80, 85, 87, 89, 94, 96, 98, 103, 105, 107, 112, 114, 116, 121, 123, 125, 130, 132, 134, 139, 141, 143, 148, 150, 152, 157, 159, 161, 166, 168, 170, 175, 177, 179, 184, 186, 188, 193, 195, 197, 202, 204, 206, 211, 213, 215, 220, 222, 224, 229, 231, 233, 238, 240, 242, 247, 249 and 251. And formulas: 254, 257, 261, 266, 271, 278, 282, 286, 255, 258, 262, 267, 272, 275, 279, 283, 287, 259, 263, 264, 268, 269, 273, 276, 280, 284, 288, are encompassed within the scope of this invention. Typically, an amino acid is substituted by a related amino acid having similar charge, hydrophobic, or stereochemical characteristics. Such substitutions would be within the ordinary skills of an artisan. Unlike in CDRs, more substantial changes can be made in FRs without adversely affecting the binding properties of an antibody. Changes to FRs include, but are not limited to engineering certain framework residues that are important for antigen contact or for stabilizing the binding site, e.g., changing the class or subclass of the constant region, changing specific amino acid residues which might alter the effector function such as Fc receptor binding (U.S. Pat. Nos. 5,624,821; 5,648,260;Lund et al. (1991) J. Immun. 147: 2657-2662; Morgan et al. (1995) Immunology 86: 319-324), or changing the species from which the constant region is derived.

One of skill in the art will appreciate that the derivatives and modifications described above are not all-exhaustive, and that many other modifications would be obvious to a skilled artisan in light of the teachings of the present disclosure.

### Nucleic Acids, Cloning and Expression Systems

The present disclosure further provides isolated nucleic acids encoding the disclosed antibodies. The nucleic acids may comprise DNA or RNA and may be wholly or partially synthetic or recombinant. Reference to a nucleotide sequence as set out herein encompasses a DNA molecule with the specified sequence, and encompasses a RNA molecule with the specified sequence in which U is substituted for T, unless context requires otherwise.

The nucleic acids provided herein comprise a coding sequence for a CDR, a H variable domain, and/or a L variable domain disclosed herein.

The present disclosure also provides constructs in the form of plasmids, vectors, phagemids, transcription or expression cassettes which comprise at least one nucleic acid encoding a CDR, a H variable domain, and/or a L variable domain disclosed here.

The disclosure further provides a host cell which comprises one or more constructs as above.

Also provided are nucleic acids encoding any CDR (CDR1, CDR2, CDR3 from either the H or L variable domain), H variable or L variable domain, as well as methods of making of the encoded products. The method comprises expressing the encoded product from the encoding nucleic acid. Expression may be achieved by culturing under appropriate conditions recombinant host cells containing the nucleic acid. Following production by expression, a H variable or L variable domain, or specific binding member may be isolated and/or purified using any suitable technique, then used as appropriate.

Antigen-binding fragments, H variable and/or L variable domains and encoding nucleic acid molecules and vectors may be isolated and/or purified from their natural environment, in substantially pure or homogeneous form, or, in the case of nucleic acid, free or substantially free of nucleic acid or genes of origin other than the sequence encoding a polypeptide with the required function.

Systems for cloning and expression of a polypeptide in a variety of different host cells are well known in the art including cells suitable for producing antibodies (Gene Expression Systems, Academic Press, eds. Fernandez et al., 1999). Briefly, suitable host cells include bacteria, plant cells, mammalian cells, and yeast and baculovirus systems. Mammalian cell lines available in the art for expression of a heterologous polypeptide include Chinese hamster ovary (CHO) cells, HeLa cells, baby hamster kidney cells, NS0 mouse myeloma cells, and many others. A common bacterial host is E. coli. Any protein expression system compatible with the invention may be used to produce the disclosed antibodies. Suitable expression systems also include transgenic animals (Gene Expression Systems, Academic Press, eds. Fernandez et al., 1999).

Suitable vectors can be chosen or constructed, so that they contain appropriate regulatory sequences, including promoter sequences, terminator sequences, polyadenylation sequences, enhancer sequences, marker genes and other sequences as appropriate. Vectors may be plasmids or viral, e.g., phage, or phagemid, as appropriate (see Sambrook et al., Molecular Cloning: A Laboratory Manual, 2.sup.nd ed., Cold Spring Harbor Laboratory Press, 1989). Many known techniques and protocols for manipulation of nucleic acid, for example, in preparation of nucleic acid constructs, mutagenesis, sequencing, introduction of DNA into cells and gene expression, and analysis of proteins, are known in the art (Current Protocols in Molecular Biology, 2.sup.nd Edition, eds. Ausubel et al., John Wiley & Sons, 1992).

The invention also provides a host cell comprising a nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction of the nucleic acid into the cells may be followed by causing or allowing expression from the nucleic acid, e.g., by culturing host cells under conditions for expression of the gene.

### Immunoconjugates

In another aspect, the antibodies of the invention can be used as a targeting agent for delivery of another therapeutic or a cytotoxic agent to a cell expressing GPNMB. The method includes administering an anti-GPNMB antibody coupled to a therapeutic or a cytotoxic agent or under conditions that allow binding of the antibody to GPNMB.

Anti-GPNMB antibodies are conjugated to a therapeutic agent, such as a cytotoxic compound, such that the resulting immunoconjugate exerts a cytotoxic or cytostatic effect on a GPNMB expressing cell. Particularly suitable moieties for conjugation to antibodies are chemotherapeutic agents, prodrug converting enzymes or toxins. For example, an anti-GPNMB antibody can be conjugated to a cytotoxic agent such as a chemotherapeutic agent (see infra) or a toxin (e.g. abrin, ricin A, pseudomonas exotoxin, or diphtheria toxin). Alternatively, anti-GPNMB antibody may be conjugated to a pro-drug converting enzyme. The pro-drug converting enzyme can be recombinantly fused to the antibody or derivative thereof or chemically conjugated thereto using known methods. Exemplary pro-drug converting enzymes are carboxypeptidase G2, β-glucuronidase, penicillin-V-amidase, penicillin-G-amidase, β-lactamase, β-glucosidase, nitroreductase and carboxypeptidase A.

Any agent that exerts a therapeutic effect on GPNMB expressing cells can be used as an agent for conjugation to an anti-GPNMB antibody of the invention. Useful classes of cytotoxic agents include, for example, antitubulin agents, auristatins, DNA minor groove binders, NDA replication inhibitors, alkylating agents (e.g., platinum complexes such as cis-plantin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated purimidines, ionophores, lexitropsins, nitrosoureas, platinols, pre-forming compounds, purine antimetabolites, puromcins, radiation sensitizers, steroids, taxanes, topoisomerase inhibitors, vinca alkaloids, or the like.

The therapeutic agent can be a cytotoxic agent. Suitable cytotoxic agents include, for example, dolastatins (e.g. auristatin E, AFP, MMAF, MMAE), DNA minor groove binders (e.g., enediynes and lexitropsins), cuocarmycins, taxanes (e.g., paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, and mitoxantrone.

In a specific embodiment, the cytotoxic or cytostatic agent is auristatin E (dolastatin-10) or a derivative thereof (e.g. an ester formed between auristatin E and a keto acid). Other typical auristatin derivatives include AFP, MMAR, and MMAE. The synthesis and structure of auristatin E and its derivates are described in U.S. Patent Application Publication No. 20030083263; PCT/US03/24209; PCT/US02/13435; and U.S. Pat. Nos. 6,323,315; 6,239,104; 6,034065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414.

In a specific embodiment anti-GPNMB antibody 1.15.1 was coupled to monomethylauristatin E via intracellular protease-sensitive valine-citrulline peptide linker (vcMMAE). Methods for making the immunoconjugate can be found in Doronina S. O. et al, 2003 Nature Biotechnology 21(7):778-794.

Techniques for conjugating therapeutic agents to proteins, and in particular, antibodies are known in the art (see, e.g. Amon et al, 1985 in Monoclonal Antibodies and Cancer Therapy, Reisfeld et al. eds., Alan R. Liss, Inc., 1985; Hellstrom et al., 1987 in Controlled Drug Delivery, Robinson et al. eds., Marcel Dekker, Inc., 2^{nd} ed. 1987; Thorpe 1985, in Monoclonal Antibodies '84: Biological and Clinical Applications, Pinchera et al. eds., EDITOR, 1985; Monoclonal Antibodies for Cancer Detection and Therapy, Baldwin et al. eds., Academic Press 1985; and Thorpe et al., 1982, Immunol. Rev. 62:119-58).

In certain embodiments of the invention, anti-GPNMB antibodies binding to GPNMB expressing cells, are internalized and accumulate in the cell. Thereby anti-GPNMB antibody immunoconjugates accumulate in GPNMB expressing cells. Typically when the anti-GPNMB antibody immunoconjugate is internalized, the agent is preferentially active. Alternatively, anti GPNMB immunoconjugates are not internalized and the drug is effective to deplete or inhibit GPNMB expressing cells by binding to the cell membrane. The therapeutic agent can be conjugated in a manner that reduces its activity unless it is cleaved off the antibody (e.g. by hydrolysis or by a cleaving agent). In this case, the agent can be attached to the antibody or derivative thereof with a cleavable linker that is sensitive to cleavage in the intracellular environment of the target but is not substantially sensitive to the extracellular environment, such that the conjugate is cleaved from the antibody or derivative thereof when it is internalized by the GPNMB expressing cell (e.g. in the endosomal or, for example by virtue of pH sensitivity or protease sensitivity, in the lysosomal environment or in a caveolea).

A therapeutic agent of the immunoconjugate can be charged relative to the plasma membrane (e.g. polarized or net charge relative to the plasma membrane), thereby further minimizing the ability of the agent to cross the plasma membrane once internalized by a cell.

The anti-GPNMB antibody immunoconjugate can comprise a linker region between the therapeutic agent and the antibody. The linker can be cleavable under intracellular conditions, such that cleavage of the linker releases the therapeutic agent from the antibody in the intracellular environment. The linker can be, e.g. a peptidyl linker that is cleaved by an intracellular peptidase or protease enzyme, including but not limited to a lysosomal or endosomal protease. Often the peptidyl linker is at least two amino acids long or at least three amino acids long. Cleaving agents can include cathepsins and D and plasmin, all of which are known to hydrolyze dipeptide drug derivative s resulting in the release of active drug inside target cells (see Dubowchik and Walker, 1999 Pharm. Therapeutics 83:67-123). Other linkers are described e.g. in U.S. Pat. No. 6,214,345.

Linkers can be pH-sensitive can often be hydrolizable under acidic conditions such as is found in the lysosome (see e.g. U.S. Pat. Nos. 5,122,368; 5,824,805; 5,622,929; Dubowchik and Walker, 1999 Pharm. Therapeutics 83:67-123; Neville et al., 1989 Biol. Chem. 264:14653-14661). Such linkers are relatively stable under neutral pH conditions, such as those in the blood, but are unstable at below pH 5.5 or 5.0, the pH of the lysosome. Linkers can be cleavable under reducing conditions (e.g. a disulfide linker) (see e.g., Thorpe et al., 1987 Cancer Res. 47:5924-5931; Wawrzynczak et al., In Immunoconjugates: Antibody Conjugates in Radioimagery and Therapy of Cancer, C. W. Vogel ed, Oxford U. Press, 1987; U.S. Pat. No. 4,880,935). The linker can be a malonate linker (Johnson et al., 1995, Anticancer Res. 15:1387-93), a maleimidobenzoly linker (lau et al., 1995, Bioorg-Med-Chem. 3(10):1299-1304) or a 3′-N-amide analog (Lau et al., 1995, Bioorg-Med-Chem.3(10):1305-1312).

### Prophylactic and Therapeutic Uses of the Present Invention

The antibodies of the invention can act as either agonists or antagonists of GPNMB, depending on the methods of their use. The antibodies can be used to prevent, diagnose, or treat medical disorders in a subject, especially in humans. Antibodies of the invention can also be used for isolating GPNMB or GPNMB-expressing cells. Furthermore, the antibodies can be used to treat a subject at risk of or susceptible to a disorder or having a disorder associated with aberrant GPNMB expression or function. Antibodies of the invention can be used to detect GPNMB in such subjects.

The present invention provides methods for treating and/or preventing a disease or disorder associated with overexpression of GPNMB and/or cell hyperproliferative disorders, particularly cancer, in a subject comprising administering an effective amount of a composition that can target cells expressing GPNMB, and inhibiting the GPNMB expression or function, and/or having therapeutic or prophylactic effects on the hyperproliferative cell disease. In one embodiment, the method of the invention comprises administering to a subject a composition comprising an immunoconjugate that comprises an antibody of the invention and a cytotoxic agent against the hyperproliferative cell disease. In another embodiment, the method of the invention comprises administering to a subject in need thereof a composition comprising a naked IgG1 antibody of the invention and one or more immunomodulators. In yet another embodiment, the method of the invention comprises administering to a subject in need thereof a composition comprising a single chain Fv antibody (anti-GPNMB) conjugated to a cytotoxic agent, or a composition comprising a bispecific antibody that have a single chain anti-GPNMB antibody component and a anti-CD3 antibody component. In a preferred embodiment, the hyperproliferative cell disease is cancer. More preferably, the cancer is melanoma, or a cancer of the CNS system, such as astrocytoma, glioblastoma, medulloblastoma, or neoplastic meningitis.

The present invention provides therapies comprising administering one of more antibodies of the invention and compositions comprising said antibodies to a subject, preferably a human subject, for preventing and/or treating a disorder characterized by or associated with aberrant expression and/or activity of GPNMB or a symptom thereof. In one embodiment, the invention provides a method of preventing or treating a disorder characterized by or associated with aberrant expression and/or activity of GPNMB or a symptom thereof, said method comprising administering to a subject in need thereof an effective amount of one or more antibodies of the invention. In certain embodiments, an effective amount of one or more immunoconjugates comprising one or more antibodies of the invention is administered to a subject in need thereof to prevent or treat a disorder characterized by or associated with aberrant expression and/or activity of GPNMB or a symptom thereof.

The invention also provides methods of preventing or treating a disorder characterized by or associated with aberrant expression and/or activity of GPNMB or a symptom thereof, said methods comprising administering to a subject in need thereof one or more of the antibodies of the invention and one or more therapies (e.g., one or more prophylactic or therapeutic agents) other than antibodies of the invention. The prophylactic or therapeutic agents of the combination therapies of the invention can be administered sequentially or concurrently. In a specific embodiment, the combination therapies of the invention comprise an effective amount of one or more antibodies of the invention and an effective amount of at least one other therapy (e.g., prophylactic or therapeutic agent) which has a different mechanism of action than said antibodies. In certain embodiments, the combination therapies of the present invention improve the prophylactic or therapeutic effect of one or more antibodies of the invention by functioning together with the antibodies to have an additive or synergistic effect. In certain embodiments, the combination therapies of the present invention reduce the side effects associated with the therapies (e.g., prophylactic or therapeutic agents).

The prophylactic or therapeutic agents of the combination therapies can be administered to a subject, preferably a human subject, in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

In a specific embodiment, a pharmaceutical composition comprising one or more antibodies of the invention described herein is administered to a subject, preferably a human, to prevent and/or treat a disorder characterized by or associated with aberrant expression and/or activity of GPNMB or a symptom thereof. In accordance with the invention, pharmaceutical compositions of the invention may also comprise one or more therapies (e.g., prophylactic or therapeutic agents), other than antibodies of the invention.

The antibodies of the invention may also be used to detect the presence of GPNMB in biological samples (in diagnostic methods or use as an efficacy marker). The amount of GPNMB detected may be correlated with the expression level of GPNMB, which, in turn, is correlated with the disease, tumor type, tumor burden or stage using methods known in the art (see for example recommendations of the AAPS Ligand Binding Assay Bioanalytical Focus Group (LBABFG) Pharm Res. 2003 November; 20(11):1885-900). Detection methods that employ antibodies are well known in the art and include, for example, ELISA, radioimmunoassay, immunoblot, Western blot, IHC, immunofluorescence, immunoprecipitation. The antibodies may be provided in a diagnostic kit that incorporates one or more of these techniques to detect GPNMB. Such a kit may contain other components, packaging, instructions, or other material to aid the detection of the protein. In a specific embodiment, the antibodies of the invention are conjugated to a radioactive isotope, and are injected to a subject to detect cells that overexpressing GPNMB.

Where the antibodies are intended for diagnostic purposes, it may be desirable to modify them, for example, with a ligand group (such as biotin) or a detectable marker group (such as a fluorescent group, a radioisotope or an enzyme). If desired, the antibodies of the invention may be labeled using conventional techniques. Suitable detectable labels include, for example, fluorophores, chromophores, radioactive atoms, electron-dense reagents, enzymes, and ligands having specific binding partners. Enzymes are typically detected by their activity. For example, horseradish peroxidase can be detected by its ability to convert tetramethylbenzidine (TMB) to a blue pigment, quantifiable with a spectrophotometer. For detection, suitable binding partners include, but are not limited to, biotin and avidin or streptavidin, IgG and protein A, and the numerous receptor-ligand couples known in the art. Other permutations and possibilities will be readily apparent to those of ordinary skill in the art, and are considered as equivalents within the scope of the instant invention.

Antibodies of the invention can be used in screening methods to identify inhibitors of GPNMB effective as therapeutics. In such a screening assay, a first binding mixture is formed by combining GPNMB and an antibody of the invention; and the amount of binding in the first binding mixture (M₀) is measured. A second binding mixture is also formed by combining GPNMB, the antibody, and the compound or agent to be screened, and the amount of binding in the second binding mixture (M₁) is measured. A compound to be tested may be another anti-GPNMB antibody. The amounts of binding in the first and second binding mixtures are then compared, for example, by calculating the M₁/M₀ ratio. The compound or agent is considered to be capable of modulating a GPNMB-associated responses if a decrease in binding in the second binding mixture as compared to the first binding mixture is observed. The formulation and optimization of binding mixtures is within the level of skill in the art, such binding mixtures may also contain buffers and salts necessary to enhance or to optimize binding, and additional control assays may be included in the screening assay of the invention. Compounds found to reduce the GPNMB-antibody binding by at least about 10% (i.e., M₁/M_{0<0.9}), preferably greater than about 30% may thus be identified and then, if desired, secondarily screened for the capacity to ameliorate a disorder in other assays or animal models as described below. The strength of the binding between GPNMB and an antibody can be measured using, for example, an enzyme-linked immunoadsorption assay (ELISA), radio-immunoassay (RIA), surface plasmon resonance-based technology (e.g., Biacore), all of which are techniques well known in the art.

The compound may then be tested in vitro as described in the Examples, infra.

### Dosage and Frequency of Administration

The amount of a prophylactic or therapeutic agent or a composition of the invention which will be effective in the prevention and/or treatment of a disorder associated with or characterized by aberrant expression and/or activity of GPNMB can be determined by standard clinical methods. For example, the dosage of the composition which will be effective in the treatment and/or prevention of cancer can be determined by administering the composition to an animal model. In addition, in vitro assays may optionally be employed to help identify optimal dosage ranges. Preliminary doses as, for example, determined according to animal tests, and the scaling of dosages for human administration is performed according to art-accepted practices. Toxicity and therapeutic efficacy can be determined by standard pharmaceutical procedures in cell cultures or experimental animals. The data obtained from the cell culture assays or animal studies can be used in formulating a range of dosage for use in humans. Therapeutically effective dosages achieved in one animal model can be converted for use in another animal, including humans, using conversion factors known in the art (see, e.g., Freireich et al. (1966) Cancer Chemother. Reports, 50(4): 219-244).

Selection of the preferred effective dose can be determined (e.g., via clinical trials) by a skilled artisan based upon the consideration of several factors which will be known to one of ordinary skill in the art. Such factors include the disease to be treated or prevented, the symptoms involved, the patient's body mass, gender, immune status and other factors known by the skilled artisan to reflect the accuracy of administered pharmaceutical compositions. Suitable regimens can be selected by one skilled in the art by considering such factors and by following, for example, dosages reported in literature and recommended in the Physician's Desk Reference (59th ed., 2005).

The precise dose to be employed in the formulation will also depend on the route of administration, and the seriousness of the cancer, and should be decided according to the judgment of the practitioner and each patient's circumstances. Effective doses may be extrapolated from dose-response curves derived from in vitro or animal model test systems.

For other cancer therapeutic agents administered to a patient, the typical doses of various cancer therapeutics are known in the art. Given the invention, certain preferred embodiments will encompass the administration of lower dosages in combination treatment regimens than dosages recommended for the administration of single agents.

In a specific embodiment, the dosage of an antibody or an immunoconjugate comprising an antibody of the invention administered to prevent and/or treat a disorder associated with or characterized by aberrant expression and/or activity of GPNMB (e.g., cancer) in a patient is 30 mg/kg or less, 25 mg/kg or less, 20 mg/kg or less, 15 mg/kg or less, preferably 12 mg/kg or less, 11 mg/kg or less, 10 mg/kg or less, 9 mg/kg or less, 8 mg/kg or less, 7 mg/kg or less, 6 mg/kg or less, 5 mg/kg or less, 4 mg/kg or less, 3 mg/kg or less, 2 mg/kg or less, or 1 mg/kg or less of a patient's body weight. In another embodiment, the dosage of an antibody or an immunoconjugate of the invention administered to prevent and/or treat a disorder associated with or characterized by aberrant expression and/or activity of GPNMB (e.g., cancer) in a patient is a unit dose of about 0.01 mg/kg to about 20 mg/kg, about 0.1 mg/kg to about 10 mg/kg, about 0.1 mg/kg to about 8 mg/kg, about 0.1 mg/kg to about 7 mg/kg, about 0.1 mg/kg to about 6 mg/kg, about 0.1 mg/kg to about 5 mg/kg, about 0.1 mg/kg to about 4 mg/kg, preferably, about 0.1 mg/kg to about 3 mg/kg, about 0.2 mg/kg to 3 mg/kg, about 0.3 mg/kg to about 3 mg/kg, about 0.4 mg/kg to about 3 mg/kg, about 0.6 mg/kg to about 3 mg/kg, about 0.8 mg/kg to about 3 mg/kg, about 0.1 mg/kg to 2 mg/kg, about 0.1 mg/kg to 1 mg/kg. In certain embodiments, the dosage of an antibody or an immunoconjugate comprising an antibody of the invention administered to prevent and/or treat a disorder associated with or characterized by aberrant expression and/or activity of GPNMB (e.g., cancer) in a patient is a unit dose of about 0.1 mg/kg, about 0.2 mg/kg, about 0.4 mg/kg, about 0.6 mg/kg, about 0.8 mg/kg, about 1.1 mg/kg, or about 1 mg/kg.

In certain embodiments, a subject is administered one or more doses of an effective amount of one or more antibodies or immunoconjugates of the invention to prevent and/or treat a disorder associated with or characterized by aberrant expression and/or activity of GPNMB, wherein the dose of an effective amount of said antibodies, immunoconjugates, compositions, or combination therapies reduces and/or inhibits proliferation of cancerous cells by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, at least 80 to 85%, at least 85% to 90%, at least 90% to 95%, or at least 95% to 98% relative to a control such as PBS in an in vitro and/or in vivo assay well-known in the art.

In other embodiments, a subject is administered one or more doses of an effective amount of one or more antibodies or immunoconjugates of the invention to prevent and/or treat a disorder associated with or characterized by aberrant expression and/or activity of GPNMB, wherein the dose of an effective amount achieves a serum titer of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the antibodies of the invention. In yet other embodiments, a subject is administered a dose of an effective amount of one or more antibodies or immunoconjugates of the invention to achieve a serum titer of at least 0.1 μg/mL, at least 0.5 μg/mL, at least 1 μg/mL, at least, 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the antibodies and a subsequent dose of an effective amount of one or more antibodies or immunoconjugates of the invention is administered to maintain a serum titer of at least 0.1 μg/mL, at least 0.5 μg/mL, at least 1 μg/mL, at least, 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL. In accordance with these embodiments, a subject may be administered 1, 2, 3, 4, 5, 6, 7, 8, 9, 10, 11, 12 or more subsequent doses.

In a specific embodiment, the invention provides methods of preventing and/or treating a disorder associated with or characterized by aberrant expression and/or activity of GPNMB, said method comprising administering to a subject in need thereof a unit dose of at least 0.01 mg/kg, at least 0.1 mg/kg, at least 0.2 mg/kg, at least 0.4 mg/kg, at least 0.6 mg/kg, at least 0.8 mg/kg, at least 1 mg/kg, or at least 1.1 mg/kg of one or more antibodies or immunoconjugates of the invention. In another embodiment, the invention provides methods of preventing and/or treating a disorder associated with or characterized by aberrant expression and/or activity of GPNMB, said method comprising administering to a subject in need thereof a unit dose of at least 0.01 mg/kg, at least 0.1 mg/kg, at least 0.2 mg/kg, at least 0.4 mg/kg, at least 0.6 mg/kg, at least 0.8 mg/kg, at least 1 mg/kg, or at least 1.1 mg/kg of one or more antibodies or immunoconjugates of the invention once every 7 days, preferably, once every 10 days, once every 12 days, once every 14 days, once every 16 days, once every 18 days, once every three weeks, or once a month. In a preferred embodiment, an immunoconjugate of the instant invention is administered at a unit dose of about 0.1 mg/kg, about 0.2 mg/kg, about 0.4 mg/kg, about 0.6 mg/kg, about 0.8 mg/kg, about 1.1 mg/kg, or about 1 mg/kg once every 10 to 20 days with 2 to 4 cycles.

The present invention provides methods of preventing and/or treating a disorder associated with or characterized by aberrant expression and/or activity of GPNMB, said method comprising: (a) administering to a subject in need thereof one or more doses of a prophylactically or therapeutically effective amount of one or more antibodies or immunoconjugates of the invention; and (b) monitoring the plasma level/concentration of the said administered antibody or antibodies in said subject after administration of a certain number of doses of the said antibody or antibodies. Moreover, preferably, said certain number of doses is 1, 2, 3, 4, 5, 6, 7, or 8 doses of a prophylactically or therapeutically effective amount one or more antibodies or immunoconjugates of the invention.

In a specific embodiment, the invention provides a method of preventing and/or treating a disorder associated with or characterized by aberrant expression and/or activity of GPNMB, said method comprising: (a) administering to a subject in need thereof a dose of at least 0.1 mg/kg (preferably at least at least 0.2 mg/kg, at least 0.4 mg/kg, at least 0.6 mg/kg, at least 0.8 mg/kg, at least 1 mg/kg, or at least 1.1 mg/kg) of one or more antibodies or immunoconjugates of the invention; and (b) administering one or more subsequent doses to said subject when the plasma level of the antibody or antibodies administered in said subject is less than 0.1 μg/mL, preferably less than 0.25 μg/mL, less than 0.5 μg/mL, less than 0.75 μg/mL, or less than 1 μg/mL. In another embodiment, the invention provides a method of preventing and/or treating a disorder associated with or characterized by aberrant expression and/or activity of GPNMB, said method comprising: (a) administering to a subject in need thereof one or more doses of at least at least 0.1 mg/kg (preferably at least at least 0.2 mg/kg, at least 0.4 mg/kg, at least 0.6 mg/kg, at least 0.8 mg/kg, at least 1 mg/kg, or at least 1.1 mg/kg) of one or more antibodies of the invention; (b) monitoring the plasma level of the administered antibody or antibodies of the invention in said subject after the administration of a certain number of doses; and (c) administering a subsequent dose of the antibody or antibodies of the invention when the plasma level of the administered antibody or antibodies in said subject is less than 0.1 μg/mL, preferably less than 0.25 μg/mL, less than 0.5 μg/mL, less than 0.75 μg/mL, or less than 1 μg/mL. Preferably, said certain number of doses is 1, 2, 3, 4, 5, 6, 7, or 8 doses of an effective amount of one or more antibodies or immunoconjugates of the invention.

Therapies (e.g., prophylactic or therapeutic agents), other than antibodies or immunoconjugates of the invention, which have been or are currently being used to prevent and/or treat a disorder associated with or characterized by aberrant expression and/or activity of GPNMB can be administered in combination with one or more antibodies or immunoconjugates of the invention according to the methods of the invention to treat and/or prevent a disorder associated with or characterized by aberrant expression and/or activity of GPNMB. Preferably, the dosages of prophylactic or therapeutic agents used in combination therapies of the invention are lower than those which have been or are currently being used to prevent and/or treat a disorder associated with or characterized by aberrant expression and/or activity of GPNMB.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same patient visit.

In certain embodiments, one or more antibodies of the invention and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

### Pharmaceutical Compositions and Methods of Administration

The disclosure provides compositions comprising anti-GPNMB antibodies. Such compositions may be suitable for pharmaceutical use and administration to patients. The compositions typically comprise one or more antibodies of the present invention and a pharmaceutically acceptable excipient. The phrase “pharmaceutically acceptable excipient” includes any and all solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. The administration may, for example, be intravenous, intraperitoneal, intramuscular, intracavity, subcutaneous or transdermal. It may also be possible to obtain compositions which may be topically or orally administered, or which may be capable of transmission across mucous membranes.

Solutions or suspensions used for intradermal or subcutaneous application typically include one or more of the following components: a sterile diluent such as water for injection, saline solution, fixed oils, polyethylene glycols, glycerin, propylene glycol, or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars; polyalcohols such as mannitol, sorbitol, and sodium chloride in the composition. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and/or by the use of surfactants. Prolonged absorption of the injectable compositions can be brought about by including in the composition an agent which delays absorption, for example, aluminum monostearate, and gelatin.

Oral compositions generally include an inert diluent or an edible carrier. They can be enclosed in gelatin capsules or compressed into tablets. For oral administration, the antibodies can be combined with excipients and used in the form of tablets, troches, or capsules. Pharmaceutically compatible binding agents, and/or adjuvant materials can be included as part of the composition. The tablets, pills, capsules, troches, and the like can contain any of the following ingredients, or compounds of a similar nature; a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, detergents, bile salts, and fusidic acid derivatives. Transmucosal administration may be accomplished, for example, through the use of lozenges, nasal sprays, inhalers, or suppositories. For example, in case of antibodies that comprise the Fc portion, compositions may be capable of transmission across mucous membranes in intestine, mouth, or lungs (e.g., via the FcRn receptor-mediated pathway as described in U.S. Pat. No. 6,030,613). For transdermal administration, the active compounds may be formulated into ointments, salves, gels, or creams as generally known in the art. For administration by inhalation, the antibodies may be delivered in the form of an aerosol spray from pressured container or dispenser, which contains a suitable propellant, e.g., a gas such as carbon dioxide, or a nebulizer.

In certain embodiments, the presently disclosed antibodies are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions containing the presently disclosed antibodies can also be used as pharmaceutically acceptable carriers. These can be prepared according to methods known to those skilled in the art, for example, as described in U.S. Pat. No. 4,522,811.

It may be advantageous to formulate oral or parenteral compositions in a dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of the composition of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

For any composition used in the present invention, the therapeutically effective dose can be estimated initially from cell culture assays. Examples of suitable bioassays include DNA replication assays, clonogenic assays and other assays as, for example, described in the Examples. The data obtained from the cell culture assays and animal studies can be used in formulating a range of dosage for use in humans. A dose may be formulated in animal models to achieve a circulating plasma concentration range that includes the IC₅₀ (i.e., the concentration of the antibody which achieves a half-maximal inhibition of symptoms). Circulating levels in plasma may be measured, for example, by high performance liquid chromatography. The effects of any particular dosage can be monitored by a suitable bioassay. The dosage lies preferably within a range of circulating concentrations with little or no toxicity. The dosage may vary depending upon the dosage form employed and the route of administration utilized.

Antibodies can be modified to become immunotoxins utilizing techniques that are well known in the art (Vitetta 1993, Immunol Today 14:252; U.S. Pat. No. 5,194,594). Cytotoxic immunoconjugates are known in the art and have been used as therapeutic agents. Such immunoconjugates may for example, use maytansinoids (U.S. Pat. No. 6,441,163), tubulin polymerization inhibitor, auristatin (Mohammad et al, 1999 Int. J. Oncol 15(2):367-72; Doronina et al, 2003 Nature Biotechnology 21(7):778-784), dolastatin derivatives (Ogawa et al, 2001 Toxicol Lett. 121(2):97-106) 21(3)778-784), Mylotarg® (Wyeth Laboratories, Philadelphia, Pa.); maytansinoids (DM1), taxane or mertansine (ImmunoGen Inc.).

Immunoradiopharmaceuticals utilizing anti-GPNMB antibodies may be prepared utilizing techniques that are well known in the art (Junghans et al. in Cancer Chemotherapy and Biotherapy 655-686 (2d edition, Chafner and Longo, eds., Lippincott Raven (1996);U.S. Pat. Nos. 4,681,581, 4,735,210, 5,101,827, 5,102,990 (RE 35,500), 5,648,471, and 5,697,902). Each of the immunotoxins and radiolabeled antibody molecules selectively kill cells expressing GPNMB. Radiolabels are known in the art and have been used for diagnostic or therapeutic radioimmuno conjugates. Examples of radiolabels include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I, ¹⁷⁷Lu, ¹⁰⁵Rh, Rhenium-186, Rhenium-188, Samarium-153, Copper-64, Scandium-47). For example, radionuclides which have been used in radioimmunoconjugate guided clinical diagnosis include, but are not limited to: ¹³¹I, ¹²⁵I, ¹²³I, ⁹⁹Tc, ⁶⁷ Ga, as well as ¹¹¹In. Antibodies have also been labeled with a variety of radionuclides for potential use in targeted immunotherapy (see Peirersz et al., 1987). These radionuclides include, for example, ¹⁸⁸Re and ¹⁸⁶Re as well as ⁹⁰Y, and to a lesser extent ¹⁹⁹Au and ⁶⁷Cu. I-(131) (see for example U.S. Pat. No. 5,460,785). Radiotherapeutic chelators and chelator conjugates are known in the art (U.S. Pat. Nos. 4,831,175, 5,099,069, 5,246,692, 5,286,850, and 5,124,471).

### EXAMPLES

The following examples, including the experiments conducted and results achieved are provided for illustrative purposes only and are not to be construed as limiting upon the present invention.

### Example 1

### Anti-GPNMB Antibodies

Anti-GPNMB antibodies were prepared as described in PCT Publication No. WO06/071441, the contents of which are hereby incorporated by reference in their entirety. Briefly, recombinant human GPNMB (SEQ ID NO:289), specifically the extra-cellular domain (ECD) was prepared for use as the immunogen. In particular, GPNMB-V5His immunogen was used as an antigen. Monoclonal antibodies against GPNMB were developed by sequentially immunizing XenoMouse® mice (XenoMouse® XMG2 strain), Abgenix, Inc. Fremont, Calif.

Hybridoma cell lines were generated from immunized mice demonstrated to have anti-GPNMB titers using standard techniques (see Mendez et al, 1997, Nat. Genet. 15:146-156).

Certain antibodies, described herein were binned in accordance with the protocol described in U.S. Patent Application Publication No. 20030157730. Results demonstrated that the monoclonal antibodies belong to distinct bins. Competitive binding by antibodies from different bins supports antibody specificity for similar or adjacent epitopes. Non competitive binding supports antibody specificity for unique epitopes.

Three bins were created to further test the binding of six anti-GPNMB antibodies. Bin 1 included GPNMB antibodies (1.2.1), (1.10.1), and (2.22.1). Bin 2 included GPNMB antibodies (2.3.1) and (1.15.1), and Bin 3 included GPNMB antibody (2.10.1). The results of the binning assays are provided below in Tables 4 and 5.

TABLE 4

| | BB | 1.1 | 1.2 | 1.3 | 1.5 | 1.7 | 1.8 | 1.9 | 1.11 | 1.12 | 1.13 | 1.15 | xV5 |
|---|---|---|---|---|---|---|---|---|---|---|---|---|---|
| BB | 0 | 16 | 58 | 24 | 6 | 25 | 14 | 9 | 8 | 9 | 7 | 15 | 32 |
| 1.1 | −16 | 0 | 57 | 16 | −29 | 34 | 9 | −35 | −9 | −7 | −24 | 35 | 28 |
| 1.2 | −42 | −16 | 0 | −60 | −89 | −49 | −81 | −75 | −73 | −65 | −81 | −43 | 45 |
| 1.3 | −11 | −33 | 8 | 0 | −75 | −40 | −49 | 171 | −29 | −33 | −67 | −73 | −15 |
| 1.5 | 25 | 35 | 64 | 60 | 0 | 20 | 10 | 24 | 17 | 27 | 12 | −8 | 61 |
| 1.7 | −1 | 76 | 65 | 20 | −8 | 0 | −8 | 4 | 4 | 6 | −3 | −3 | 95 |
| 1.8 | −7 | 29 | 45 | 35 | −3 | −7 | 0 | 4 | −1 | 0 | −6 | 3 | 52 |
| 1.9 | −5 | 18 | 47 | −7 | −10 | 3 | 4 | 0 | 4 | 5 | −5 | −1 | 17 |
| 1.11 | 18 | 40 | 60 | 29 | −11 | 1 | 15 | 16 | 0 | 8 | 5 | −23 | 48 |
| 1.12 | −10 | 26 | 43 | 27 | −5 | 3 | −12 | −4 | −12 | 0 | −9 | −13 | 57 |
| 1.13 | 1 | 30 | 40 | 27 | 2 | 9 | 2 | 10 | 11 | 17 | 0 | −13 | 59 |
| 1.15 | −19 | 91 | 79 | 71 | 15 | 21 | 8 | 12 | 10 | 15 | 13 | 0 | 89 |
| xV5 | 41 | 134 | 239 | 46 | 5 | 443 | 230 | −1 | 70 | 257 | 24 | 535 | 0 |
| | I | II | III | IV | V | VI | VII | VIII | | IX | | | |
| | 1.1 | 1.2 | 1.3 | 1.5 | 1.7 | 1.8 | 1.9 | 1.15 | | xV5 | | | |
| | | | | 1.13 | | 1.11 | | | | | | | |
| | | | | | | 1.12 | | | | | | | |

TABLE 5

| | 1.1 | 1.2 | 1.3 | 1.5 | 1.7 | 1.8 | 1.9 | 1.11 | 1.12 | 1.13 | 1.15 | xV5 | BB |
|---|---|---|---|---|---|---|---|---|---|---|---|---|---|
| 1.1 | 0 | 72 | 39 | −36 | 49 | 8 | −14 | −3 | 18 | −14 | 35 | 28 | −2 |
| 1.2 | 10 | 0 | −60 | −103 | −46 | −64 | −76 | −71 | −69 | −83 | −74 | 44 | −46 |
| 1.3 | −49 | −9 | 0 | −111 | −88 | −78 | 281 | −66 | −57 | −93 | −115 | −89 | −33 |
| 1.5 | 61 | 106 | 77 | 0 | 13 | 28 | 17 | 20 | 40 | 2 | −3 | 87 | 19 |
| 1.7 | 94 | 77 | 51 | −25 | 0 | −9 | −3 | 12 | 4 | −4 | −17 | 96 | 17 |
| 1.8 | 42 | 71 | 74 | −24 | 2 | 0 | −9 | 1 | −1 | −12 | −5 | 61 | 4 |
| 1.9 | 14 | 74 | 28 | −24 | 6 | 4 | 0 | 3 | 5 | −13 | 8 | 16 | −17 |
| 1.11 | 59 | 66 | 77 | −20 | 3 | −5 | 13 | 0 | 11 | −9 | −5 | 92 | 21 |
| 1.12 | 84 | 67 | 61 | −36 | −12 | −8 | −6 | −4 | 0 | −16 | −34 | 95 | 12 |
| 1.13 | 74 | 93 | 49 | −12 | 22 | 12 | 23 | 21 | 19 | 0 | 20 | 98 | 55 |
| 1.15 | 127 | 90 | 51 | −9 | 17 | 12 | 19 | 19 | 21 | 5 | 0 | 125 | 59 |
| xV5 | 189 | 330 | 22 | 14 | 611 | 376 | −17 | 113 | 445 | 44 | 750 | 0 | 100 |
| BB | 25 | 73 | 65 | 3 | 34 | 23 | 14 | 19 | 22 | 13 | 39 | 44 | 0 |
| | | | | I | II | III | IV | V | VI | VII | | VIII | |
| | | Cut-off = 100 | | 1.1 | 1.2 | 1.3 | 1.5 | 1.7 | 1.9 | 1.15 | | xV5 | |
| | | | | | | | | 1.8 | | | | | |
| | | | | | | | | 1.11 | | | | | |
| | | | | | | | | 1.12 | | | | | |
| | | | | | | | | 1.13 | | | | | |
| | | | | I | II | III | IV | V | VI | VII | VIII | | IX |
| | | Cut-off = 90 | | 1.1 | 1.2 | 1.3 | 1.5 | 1.7 | 1.8 | 1.9 | 1.15 | | xV5 |
| | | | | | | | 1.13 | | 1.11 | | | | |
| | | | | | | | | | 1.12 | | | | |

Fully human monoclonal antibodies (mAb)-IgG2 to CG56972/GPNMB, an antigen predominantly found on the surface of melanoma and brain tumor cells, were generated. The naked CR011 IgG2 mAb (mAb 1.15) had no effect on CG56972 expressing cells either in vitro or in vivo. Thus it was examined whether isotype switching from an IgG2 to an IgG1 might enable the mAb to kill human melanoma cells through ADCC effector functions.

Briefly, to switch CR011 from an IgG2 to IgG1 antibody, double stranded DNA encoding constant region of IgG1 (allotype Gm(f)) was synthesized, and IgG2 constant region was replaced with IgG1 constant region using overlapped PCR approach. The sequences are described below:

### CR011 mAb 1.15.1 Mature Heavy Chain (IgG2):

(SEQ ID NO: 290)
QVQLQESGPGLVKPSQTLSLTCTVSGGSISSFNYYWSWIRHHPGKGLEW
IGYIYYSGSTYSNPSLKSRVTISVDTSKNQFSLTLSSVTAADTAVYYCA
RGYNWNYFDYWGQGTLVTVSSASTKGPSVFPLAPCSRSTSESTAALGCL
VKDYFPEPVTVSWNSGALTSGVHTFPAVLQSSGLYSLSSVVTVPSSNFG
TQTYTCNVDHKPSNTKVDKTVERKCCVECPPCPAPPVAGPSVFLFPPKP
KDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPRE
PQVYTLPPSREEMTKNQVSLTCLVKGFYPSDIAVEWESNGQPENNYKTT
PPMLDSDGSFFLYSKLTVDKSRWQQGNVFSCSVMHEALHNHYTQKSLSL
SPGK

### CR011 mAb 1.15.1 Mature Heavy Chain (IgG1):

(SEQ ID NO: 291)
QVQLQESGPGLVKPSQTLSLTCTVSGGSISSFNYYWSWIRHHPGKGLEW
IGYIYYSGSTYSNPSLKSRVTISVDTSKNQFSLTLSSVTAADTAVYYCA
RGYNWNYFDYWGQGTLVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLF
PPKPKDTLMISRTPEVTCVVVDVSHEDPEVKFNWYVDGVEVELNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK

Generation of Fully Human CR011 Monoclonal Antibodies to CG56972/GPNMB:

The CG56972 protein is predicted to be a type I transmembrane glycoprotein. The highly elevated expression of CG56972 transcripts and the potential cell surface localization of this protein in human cancer samples encouraged us to generate monoclonal antibodies (mAbs) as a potential cancer therapeutic. Therefore, the human CG56972 extracellular domain (ECD; aa 23-480) was cloned. Sequencing of the cloned cDNA revealed the presence of an in-frame 36-nt insertion, likely due to alternative splicing at the exon 6/7 boundary, which added an additional 12-aa (ATTLKSYDSNTP) (SEQ ID NO: 292) after residue 339 of the published GPNMB protein sequence. The authenticity of 36-nt insertion was verified via RT-PCR. The cDNA was next expressed in human HEK293 cells. The resultant protein was harvested, purified from the conditioned media and used as an immunogen to generate fully human mAbs against CG56972-ECD. Following immunization of XenoMouse®, mAbs that specifically recognized the CG56972-ECD protein via ELISA were generated. The lead mAb, designated 1.15 or CR011 against purified CG56972-ECD, exhibiting a Kd of 52 nM against purified CG56972-ECD protein, was selected for in depth characterization and will be the focus of the remainder of this example.

Generation of Immunoconjugates:

CR011AE is an antibody-drug conjugate composed of the anti-GPNMB (CG56972) fully human antibody CR011 conjugated with the toxin Auristatin E through a protease-cleavable linker. The toxin-to-antibody ratio is approx. 4.0 but may vary between 3.5 and 4.2. While the CR011 antibody is IgG2, it is therefore possible to append up to 12 toxin molecules per antibody molecule using the free thiols as a reactive site.

The structure of Maleimidocoaproyl-Valine-Citrullin-Monomethyl-Auristatin E (vcMMAE) is shown in FIG. 8

Conjugation: A process of generating the drug-substance consisting of CR011 mAb with VCMMAE attached. An overview of the conjugation process is summarized in FIG. 9

Briefly, the conjugation process for CR011 fully human antibody consists of the following 4 steps. 1) Buffer exchange and sucrose removal by diafiltration, 2) Disulfides reduction, 3) Conjugation to vcMMAE and finally, 4) Purification of conjugated CR011-vcMMAE by diafiltration. There are several assays throughout the process, i.e. in-process assays, which include Ellman's assay and determination of protein concentration. At the end of the process, the drug substance, i.e. the conjugate, is analyzed for drug-to-antibody ratio, free drug content and protein concentration.

Diafiltration of the Bulk Antibody:

The bulk antibody originally formulated in phosphate pH 7-10% sucrose was buffer exchanged into the conjugation buffer (borate pH 9.0-NaCl) by diafiltration over 10 diavolumes. At the end of diafiltration, CR011 was diluted to ˜5.5 mg/mL and filtered through a set of two filters consisting of 1.2 and 0.22 μm. The buffer exchange is required because sucrose interferes with reduction. In addition, high pH improves CR011 solubility.

CR011 Reduction—General Considerations:

CR011 is produced as an IgG2 isotype product and contains 6 disulfide bridges in the hinge region. These disulfides can be reduced under mild conditions to give rise to 12 cysteine residues. Therefore, it is possible to maximally attach 12 vcMMAE drug molecules per antibody. For the process, however, the bulk antibody is only partially reduced because the aim is to generate conjugates with an average of 4 vcMMAE molecules. The reason for this is two-fold. First, it broadens the therapeutic window by decreasing potential systemic toxicity associated with MMAE. Second, it is difficult and sometimes impossible to produce fully-loaded conjugates with low aggregation because of greatly reduced solubility imparted by the hydrophobic drug.

Process:

Tris-(carboxyethyl)-phosphine or TCEP was added at the 4:1 molar ratio (TCEP:mAb) to CR011 at a concentration of ˜5.5 mg/mL in the jacketed reactor equipped with an agitator set to 90 RPM. The reaction was allowed to proceed for 3 hours at 37° C. in the presence of 1 mM EDTA. At the end, Ellman's assay was used to determine the amount of free thiols. Typically, it was 4.2 thiols per antibody. The reactor was then chilled to 4° C.

CR011 Conjugation—General Considerations:

TCEP was not fully consumed during the reduction. The left over TCEP was capable of reacting with vcMMAE. However, this spurious side reaction was slower compared to the conjugation reaction and can be mitigated by adding an excess of vcMMAE. The advantage of TCEP compared to DTT is that it does not require removal of the left-over reducing agent.

Process:

vcMMAE was dissolved in DMSO and added at 20% molar excess to the reduced CR011 mAb. The reaction was allowed to proceed for 1 hour. The final concentration of DMSO is 4% (v/v). DMSO played a dual purpose in the process. It is required for solubilizing the drug and also it helps to solubilize the conjugate. At the end of conjugation, N-acetylcysteine was added to quench any unreacted drug.

CR011-vcMMAE Purification:

The temperature in the reactor was brought to room temperature. A 40% sucrose stock solution was used to adjust the final sucrose concentration to 10% (w/v) followed by a pH adjustment using 300 mM histidine HCl pH 5.0 buffer to a final pH of 6.0. The conjugate was then purified by diafiltration into 20 mM histidine pH 6.0-10% sucrose (w/v) buffer and using 10 diavolumes. At the end of diafiltration, the conjugate was concentrated to ˜7 mg/mL and filtered through a set of three filters consisting of 1.2, 0.45 and finally, 0.22 μm.

CR011-vcMMAE Formulation:

The conjugate was formulated by adding Tween-20 to a final concentration of 0.02% and by diluting to 6 mg/mL (±10%) using formulation buffer (20 mM histidine pH 6.0, 10% sucrose, 0.02% Tween-20). The conjugate was then stored at 4° C. until pooling if more than one lot is being manufactured (a.k.a. staging time). After pooling, the final concentration was adjusted to 5.0 mg/mL (±5%) and the drug substance was stored frozen.

### Example 2

### Materials and Methods

Cell Lines and Reagents:

Cancer cell lines were of human origin and were obtained from the American Type Culture Collection (Manassas, Va.) and from the National Cancer Institute (Bethesda, Md.) and cultured in RPMI-1640 supplemented with 10% FBS (growth media). Sources of reagents were as follows: RAFKi (553013), MEKi (U0126), MEKi (PD98059), MEKi (444939), ERKi (FR180204), p38 MAPKi (SB202190), p38 MAPKi (SB203580), aurora kinase inhibitor (189404), JNK inhibitor (420119), MMPi (GM6001) were from EMD Chemicals (San Diego, Calif.). ERKi (A6355), RAFKi: (GW5074), ammonium chloride (NH₄Cl), chloroquine, monensin, cyclohexamide and emetine were from Sigma (Saint Louis, Mo.). HSP90i (geldanamycin) was from AG Scientific (San Diego, Calif.) and imatinib (Gleevec) was purchased from a pharmacy.

Immunoblotting:

Cells were seeded in 6-well tissue culture dishes at a subconfluent density and allowed to attach overnight. The following day, cells were treated with the compounds diluted in growth media, as indicated in the figures/legends. For harvesting cell lysates, media was removed, cells were briefly washed with serum-free media and then 1× Tris-glycine SDS sample buffer (Invitrogen, Carlsbad, Calif.) supplemented with 10 mM dithiothreitol (Sigma) was added directly to the wells. Cell lysates were then collected and boiled for 10 minutes. For preparing conditioned media, drug treatments were performed in a minimum volume of growth media (1.5 mL/well). Following 48 hours of incubation, conditioned media was collected, briefly microfuged, mixed with an equal volume of 2× Tris-glycine SDS sample buffer (Invitrogen, Carlsbad, Calif.) supplemented with 20 mM dithiothreitol (Sigma) and boiled for 10 minutes. For cell lysates, samples were quantified and an equal quantity of protein/lane was resolved on 4-20% gradient polyacrylamide gels (Invitrogen) and then transferred to nitrocellulose filters (Invitrogen). The volume of conditioned media loaded/lane was adjusted based on the protein concentration determined in the respective cell lysates. Immunoblotting was performed using standard procedures and the following primary antibodies: MARTI (sc-20032), TYRP2 (sc-25544), TYRP1 (sc-25543), PMEL17 (sc-15010), MAGEA1 (sc-20033), MCSP (sc-20162), MTf (sc-26651), MCAM (sc-18942), pp-ERK (sc-7976) and ERK (sc-94) (preceding antibodies were purchased from Santa Cruz Biotechnologies, Santa Cruz, Calif.), GPNMB (AF2550; R & D Systems, Minneapolis Minn.), actin (A5060; Sigma). Following incubation with the appropriate horseradish peroxidase-conjugated secondary antibodies, enhanced chemiluminescence (GE Healthcare, Chalfont St. Giles, United Kingdom) was used for detection.

Flow Cytometry:

Cells were seeded in 6-well tissue culture dishes at a subconfluent density and allowed to attach overnight. The following day, cells were treated as indicated in the figures/legends, harvested with versene (Invitrogen), washed with PBS and incubated with primary antibody (10 μg/mL) diluted in staining buffer (PBS pH 7.4, 4% FBS, 0.1% sodium azide) for 30 minutes on ice. Primary antibodies utilized were CR011 (human IgG2 monoclonal antibody raised to the extracellular domain of human GPNMB (Tse, K. F., et al., CR011, a fully human monoclonal antibody-auristatin E conjugate, for the treatment of melanoma. Clin Cancer Res, 12: 1373-1382, 2006)) and an unrelated human IgG2 isotype control monoclonal antibody. After incubation with primary antibody, samples were washed with staining buffer and then incubated with secondary antibody (PE-conjugated anti-human antibody; 1:100) for 30 minutes on ice. After incubation, samples were washed with staining buffer and examined on a Becton Dickinson FACSCalibur flow cytometer (BD Immunocytometry Systems, San Jose, Calif.). Data analysis was performed with Becton Dickinson Cell Quest software and the geometric mean fluorescence intensity (GeoMean) was determined for each sample.

In Vitro Growth-Inhibition/Cytotoxicity Assay:

Cells were seeded in tissue culture dishes at a subconfluent density and allowed to attach overnight. The following day, cells were treated with or without U0126 (1 μM) diluted in growth media and incubated for 48 hours. After incubation, cells were washed, harvested, counted and seeded in growth media in 6-well tissue culture dishes in the absence or presence of CR011-vcMMAE at various concentrations for 72 hours. CR011-vcMMAE is a GPNMB-targeting ADC which has been previously described (Tse, K. F., et al., CR011, a fully human monoclonal antibody-auristatin E conjugate, for the treatment of melanoma. Clin Cancer Res, 12: 1373-1382, 2006). After incubation, photomicrographs were taken and adherent cells were trypsinized, mixed with trypan blue dye and counted on a haemocytometer. Only viable cells which excluded trypan blue were counted. For SKMEL2, cells were seeded in 96-well plates after U0126 treatment and assayed after 72 h using the CellTiter-Glo assay according to the manufacturer's instructions (Promega, Madison, Wis.).

### Example 3

### Expression of GPNMB in Melanoma and Glioblastoma Cell Lines

GPNMB is expressed in melanoma and glioblastoma cell lines and exhibits a unique expression profile compared to other melanoma-associated targets.

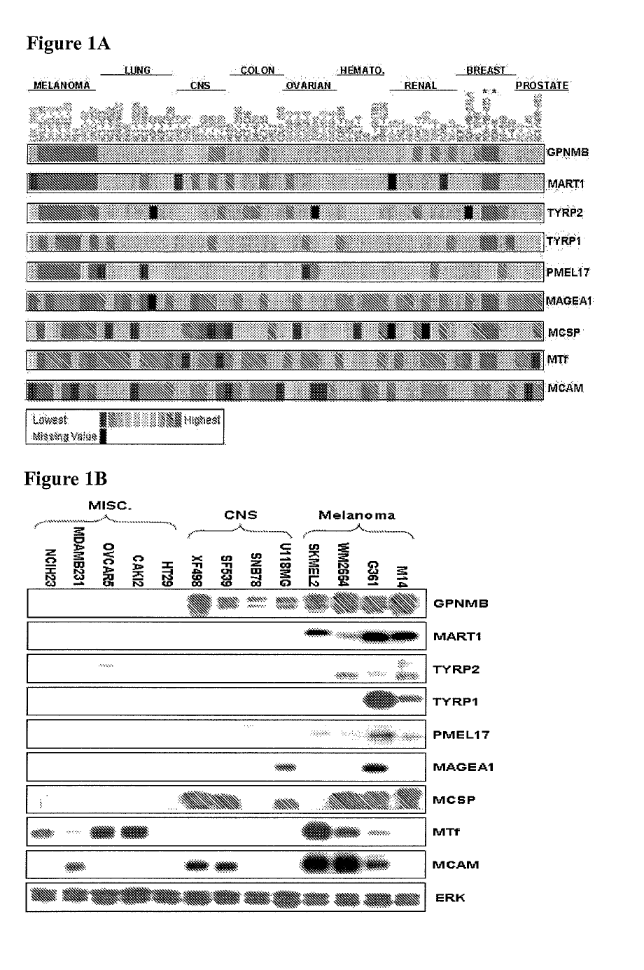

A microarray analysis of the transcriptional profile of GPNMB on the NCI-60 cancer cell line panel revealed that GPNMB was most consistently expressed in cell lines derived from melanoma (9/10) and CNS (4/6) tumors (FIG. 1A). The selective expression of GPNMB in melanoma and CNS (particularly glioblastoma) cell lines was confirmed at the protein level by immunoblotting, where GPNMB migrated as a doublet of ˜130 and 110 kDa (FIG. 1B). In addition to GPNMB, the expression of 8 other melanoma-associated molecules that are under consideration as potential targets for melanoma therapy was examined (FIG. 1A, B). In comparison to the other melanoma targets examined, GPNMB exhibited a unique expression profile that was consistent with its potential utility for the targeted therapy of melanoma and glioblastoma.

### Example 4

### Induction of GPNMB Expression Using Inhibitors of the ERK Signaling Pathway

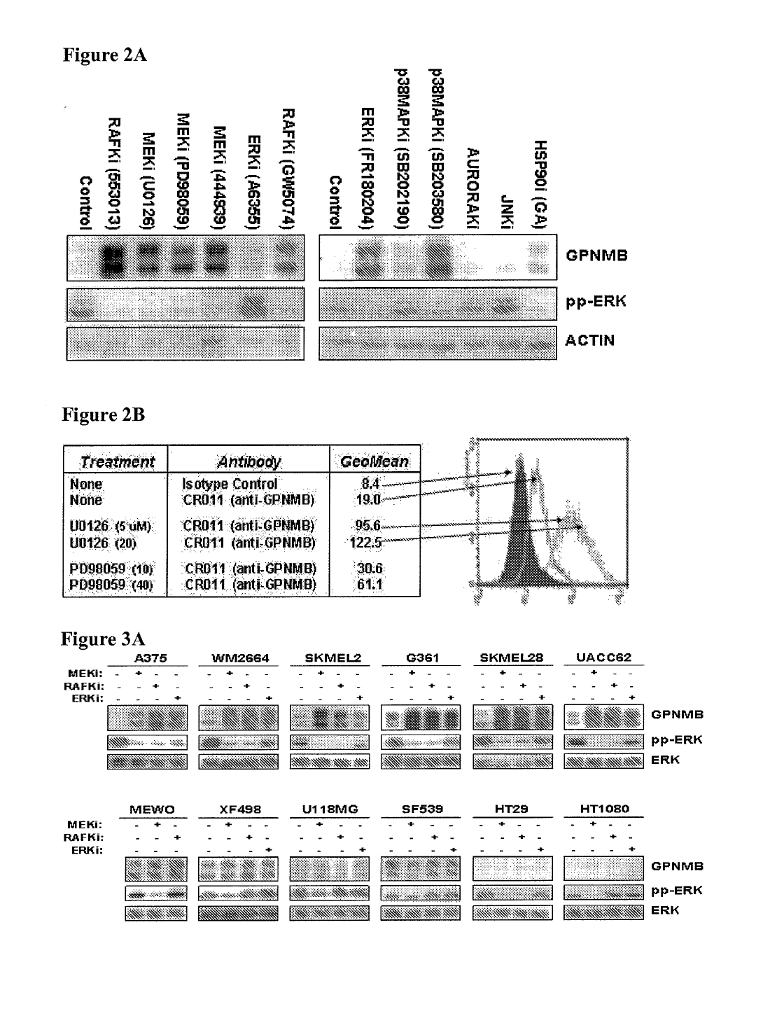

GPNMB expression has been shown to be both necessary and sufficient for the activity of the CR011-vcMMAE (Tse, K. F., et al., CR011, a fully human monoclonal antibody-auristatin E conjugate, for the treatment of melanoma. Clin Cancer Res, 12: 1373-1382, 2006), and thus it is likely that the activity of this ADC is influenced by the level and consistency of GPNMB expression on tumor cells. Since tumors often exhibit heterogeneous expression of potential therapeutic targets, a screen was performed to attempt to identify compounds which increased the expression of GPNMB with the idea that such compounds might be used therapeutically to increase the anticancer activity of CR011-vcMMAE. The A375 melanoma cell line was initially employed since this cell line harbors a mutant form of BRAF and thus represents a common molecularly-defined subclass of melanoma. Moreover, this cell line expresses a very low basal level of GPNMB which facilitates the detection of potentially weak GPNMB inductions. A variety of compounds were surveyed for their ability to increase GPNMB expression as detected by immunoblotting, and a representative experiment is presented in FIG. 2A. In this particular experiment, a number of compounds were found to increase GPNMB expression, the majority of which were inhibitors of the ERK signaling pathway. This included inhibitors of RAF, MEK and ERK, in addition to the HSP90 inhibitor geldanamycin, which is known to inhibit the ERK pathway due to the dependency of BRAF on HSP90 function (Roberts, P. J. and Der, C. J. Targeting the Raf-MEK-ERK mitogen-activated protein kinase cascade for the treatment of cancer. Oncogene, 26: 3291-3310, 2007). (Note that although the induction of GPNMB by geldanamycin was relatively weak in the experiment presented in FIG. 2A in which cells were exposed to compound for just 24 hours; more robust GPNMB induction was observed in additional experiments in which A375 and other melanoma cells (i.e. WM2664) were exposed to geldanamycin for 48 hours).

In the experiment presented in FIG. 2A, potential inhibition of the ERK pathway was investigated by immunoblotting for phospho-ERK (pp-ERK), which represents the active form of this signaling molecule. The results of this analysis demonstrated that, as expected, ERK phosphorylation/activation was indeed inhibited by compounds which targeted various components of this signaling pathway.

Some of the compounds which were found to increase GPNMB expression in A375 cells as indicated by immunoblotting were also examined for their ability to increase the surface expression of GPNMB in these cells via flow cytometry (FIG. 2B). The results of this analysis demonstrated that the increase in overall GPNMB expression that was evident via immunoblotting was also reflected by increased GPNMB at the cell surface.

Not all compounds that were examined in this screen increased the expression of GPNMB. For example, inhibitors of JNK and aurora kinases were ineffective via immunoblotting (FIG. 2A). In addition, numerous other compounds such as dacarbazine, IFNα, IFNγ, carmustine, cisplatin, paclitaxel and inhibitors of mTOR (rapamycin), PI3K (LY94002), IκB (BMS345541), methyltransferase (5-AZA, decitabine), histone deacetylase (belinostat) and the proteasome (bortezomib) were ineffective as determined by immunoblotting and/or flow cytometry analysis.

### Example 5

### Influence of NRAS/BRAF Mutational Status and Tumor Type on Induction of GPNMB Expression by Inhibitors of the ERK Signaling Pathway

To examine whether GPNMB induction by ERK-pathway inhibitors was influenced by tumor type and/or NRAS/BRAF mutational status, a variety of cell lines representing melanomas and non-melanomas with or without mutations in NRAS or BRAF (Table 36; (Abi-Habib, R. J., et al., BRAF status and mitogen-activated protein/extracellular signal-regulated kinase kinase 1/2 activity indicate sensitivity of melanoma cells to anthrax lethal toxin. Mol Cancer Ther, 4: 1303-1310, 2005; Gupta, S., et al., Dissection of Ras-dependent signaling pathways controlling aggressive tumor growth of human fibrosarcoma cells: evidence for a potential novel pathway. Mol Cell Biol, 20: 9294-9306, 2000; Ikediobi, O. N., et al., Mutation analysis of 24 known cancer genes in the NCI-60 cell line set. Mol Cancer Ther, 5: 2606-2612, 2006; and Lev, D. C., et al., Exposure of melanoma cells to dacarbazine results in enhanced tumor growth and metastasis in vivo. J Clin Oncol, 22: 2092-2100, 2004)) were exposed to ERK-pathway inhibitors and examined for GPNMB expression via immunoblotting (FIG. 3A).

TABLE 36
Cell Line Characterization

| | | BRAF/NRAS |
|---|---|---|
| Cell Line | Cancer Type | Mutations |
| A375 | Melanoma | BRAF |
| WM2664 | Melanoma | BRAF |
| M14 | Melanoma | BRAF |
| G361 | Melanoma | BRAF |
| SKMEL28 | Melanoma | BRAF |
| UACC62 | Melanoma | BRAF |
| SKMEL2 | Melanoma | NRAS |
| MEWO | Melanoma | NO |
| SF539 | Glioblastoma | NO |
| U118MG | Glioblastoma | Unknown |
| XF498 | Glioblastoma | Unknown |
| HT29 | Colon | BRAF |
| HT1080 | Fibrosarcoma | NRAS |

This analysis showed that melanomas harboring mutations in either NRAS (SKMEL2) or BRAF (A375, WM2664, G361, SKMEL28, UACC62) exhibited an induction of GPNMB following exposure to inhibitors of the ERK pathway, while non-melanomas harboring mutations in NRAS (HT1080) or BRAF (HT29) did not. In addition, neither melanoma (MEWO) nor non-melanoma (SF539 glioblastoma) cell lines possessing wild-type NRAS/BRAF exhibited GPNMB induction following exposure to ERK pathway inhibitors. This data indicates that the induction of GPNMB in a particular cell line by inhibitors of the ERK pathway is dependent upon the presence of a mutation in NRAS or BRAF in addition to the proper cellular context/tumor type.

Next, the effect of kinetics and drug concentration on the induction of GPNMB by inhibitors of MEK, RAF and ERK was examined (FIG. 3B). This experiment confirmed that these compounds induced GPNMB expression in melanoma cell lines harboring mutations in NRAS (SKMEL2) or BRAF (A375), but not in a non-melanoma cell line possessing wild-type NRAS/BRAF (SF539 glioblastoma). Results obtained with a MEK inhibitor (FIG. 3B, top) showed that the level of GPNMB induction increased with exposure time and in some instances exhibited dose-responsiveness. As indicated by phospho-ERK levels, the MEK inhibitor exhibited robust and sustained inhibition of the ERK pathway in A375 and SKMEL2 cell lines, and transient inhibition in SF539 cells. Results using a RAF inhibitor (FIG. 3B, middle) were similar to those found with the MEK inhibitor, although it is interesting to note that the RAF inhibitor showed no evidence of phospho-ERK inhibition in SF539 cells and thus this compound appears to exhibit selectivity for mutant BRAF (also see FIG. 3A). Finally, exposure of cells to an ERK inhibitor showed strong induction of GPNMB in A375 cells, but only following an exposure time of 48 hours (FIG. 3B, bottom). Little or no GPNMB induction was seen in SKMEL2 or SF539 cells. This compound produced a relatively weak inhibition of the ERK pathway as indicated by an examination of phospho-ERK levels. Consistent with the immunoblotting results, analysis by flow cytometry demonstrated that the MEK, RAF and ERK inhibitors induced GPNMB surface expression in a melanoma (A375), but not in a glioblastoma (SF539) cell line (Table 37, top).

TABLE 37
Flow Cytometry Analysis

| | | A375 (GeoMean) | | | SF549 (GeoMean) | | |
|---|---|---|---|---|---|---|---|
| Treatment | Antibody | 12 h | 24 h | 48 h | 12 h | 24 h | 48 h |
| None | Isotype Control | 7.4 | 7.5 | 7.8 | 8.2 | 8.7 | 9.5 |
| None | CR011 (anti-GPNMB) | 12.4 | 13.1 | 11.6 | 36.2 | 29.3 | 36.5 |
| MEKi (1.3 uM) | CR011 (anti-GPNMB) | 14.2 | 15.0 | 33.2 | 39.4 | 30.9 | 34.0 |
| MEKi (5 um) | CR011 (anti-GPNMB) | 14.7 | 16.4 | 52.4 | 41.5 | 36.3 | 41.5 |
| MEKi (20 um) | CR011 (anti-GPNMB) | 18.3 | 22.4 | 84.0 | 46.3 | 39.2 | ND |
| RAFKi (0.63 uM) | CR011 (anti-GPNMB) | 12.3 | 13.0 | 30.6 | 28.8 | 25.6 | 25.5 |
| RAFKi (2.5 uM) | CR011 (anti-GPNMB) | 13.0 | 16.0 | 54.8 | 30.0 | 24.6 | 30.8 |
| RAFKi (10 um) | CR011 (anti-GPNMB) | 20.3 | 43.5 | 77.7 | 31.8 | 24.3 | 30.9 |
| ERKi (3.1 uM) | CR011 (anti-GPNMB) | 13.0 | 13.5 | 13.1 | 32.6 | 33.7 | 32.6 |
| ERKi (12.5 uM) | CR011 (anti-GPNMB) | 13.0 | 13.0 | 18.7 | 33.8 | 31.0 | 30.0 |
| ERKi (50 um) | CR011 (anti-GPNMB) | 13.0 | 14.9 | 50.3 | 31.3 | 30.7 | 30.6 |

| Treatment | Antibody | SKMEL2 | Treatment | Antibody | WM2664 |
|---|---|---|---|---|---|
| None | Isotype Control | 6.9 | None | Isotype Control | 7.3 |
| None | CR011 (anti-GPNMB) | 60.5 | None | CR011 (anti-GPNMB) | 37.2 |
| MEKi | CR011 (anti-GPNMB) | 174.5 | MON (500 nM) | CR011 (anti-GPNMB) | 86.8 |
| Imatinib | CR011 (anti-GPNMB) | 103.7 | MON (250 nM) | CR011 (anti-GPNMB) | 88.9 |
| p38i | CR011 (anti-GPNMB) | 85.1 | MON (125 nM) | CR011 (anti-GPNMB) | 87.1 |
| NH₄Cl | CR011 (anti-GPNMB) | 93.8 | MMPi (100 uM) | CR011 (anti-GPNMB) | 66.8 |
| CLQ | CR011 (anti-GPNMB) | 91.2 | MMPi (50 uM) | CR011 (anti-GPNMB) | 68.9 |

| | | |
|---|---|---|
| MMPi (25 uM) | CR011 (anti-GPNMB) | 55.9 |

For the data shown in Table 37, flow cytometry was performed on intact, non-permeabilized cells using the indicated primary monoclonal antibodies. The CR011 antibody utilized was not MMAE-conjugated, but is the same antibody used to generate CR011-vcMMAE. The results are reported as GeoMeans. For the data shown in the top panel of Table 37, A375 (melanoma) or SF539 (glioblastoma) cells were treated with the indicated concentrations of MEKi (U0126), RAFKi (553013) or ERKi (FR180204) for 12, 24 or 48 hours prior to analysis. For the data shown in the bottom left panel of Table 37, SKMEL2 (melanoma) cells were treated with MEKi (U0126; 10 uM), imatinib (20 uM), p38i (p38 MAPKi SB202190; 50 uM), NH₄Cl (ammonium chloride; 20 mM) or CLQ (chloroquine; 20 uM) for 48 hours prior to analysis. For the data shown in the bottom right panel of Table 37, WM2664 (melanoma) cells were treated with MON (monensin) or MMPi (GM6001) for 48 hours prior to analysis.

### Example 6

### Sensitization of Melanoma Cells to Growth-Inhibitory Activity of CR011-vcMMAE Using Inhibitors of the ERK Signaling Pathway

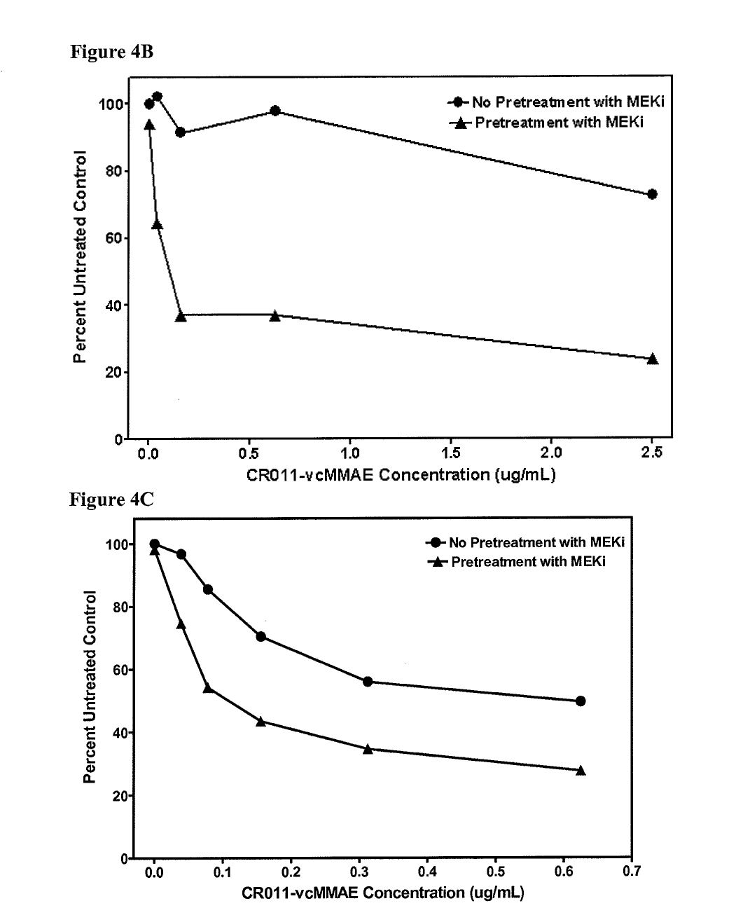

Having established that inhibitors of the ERK pathway enhance overall and cell-surface GPNMB expression in melanoma cell lines harboring mutant NRAS or BRAF, studies were then designed to determine whether this translated into increased sensitivity to the growth-inhibitory effects of the GPNMB-targeting ADC, CR011-vcMMAE. To this end, the effect of exposing melanoma cells to a MEK inhibitor prior to the addition of CR011-vcMMAE was examined. The UACC62 melanoma cell line was chosen for this analysis since these cells harbor mutant BRAF, exhibit relatively low GPNMB surface expression (Tse, K. F., et al., CR011, a fully human monoclonal antibody-auristatin E conjugate, for the treatment of melanoma. Clin Cancer Res, 12: 1373-1382, 2006), are relatively insensitive to growth-inhibition by CR011-vcMMAE, and are highly responsive to GPNMB induction by inhibitors of the ERK pathway (FIG. 3A). Following pretreatment without or with a non-toxic dose of a MEK inhibitor, UACC62 cells were incubated with various doses of CR011-vcMMAE for 3 days after which time cultures were photographed (FIG. 4A) and live cells were quantified by trypan blue dye exclusion (FIG. 4B). The results of this experiment indicated that pretreatment with a MEK inhibitor sensitized melanoma cells to the growth-inhibitory activity of CR011-vcMMAE. For example, little growth-inhibition was mediated by CR011-vcMMAE (0.16 μg/mL) on UACC62 cells that were not pretreated with the MEK inhibitor, while strong growth-inhibition was mediated by the same dose of CR011-vcMMAE used on cells that were pretreated with the MEK inhibitor. Sensitization of UACC62 cells to CR011-vcMMAE by pretreatment with the MEK inhibitor was confirmed using another measure of cell viability (CellTiter-Glo assay which measures ATP). In addition, a RAF inhibitor (553013) was also found to sensitize UACC62 cells to CR011-vcMMAE. Pretreatment with a MEK inhibitor also sensitized SKMEL2 cells to CR011-vcMMAE (FIG. 4C).

### Example 7

### Enhanced Expression of Melanoma-Associated Targets Using Inhibitors of the ERK Signaling Pathway

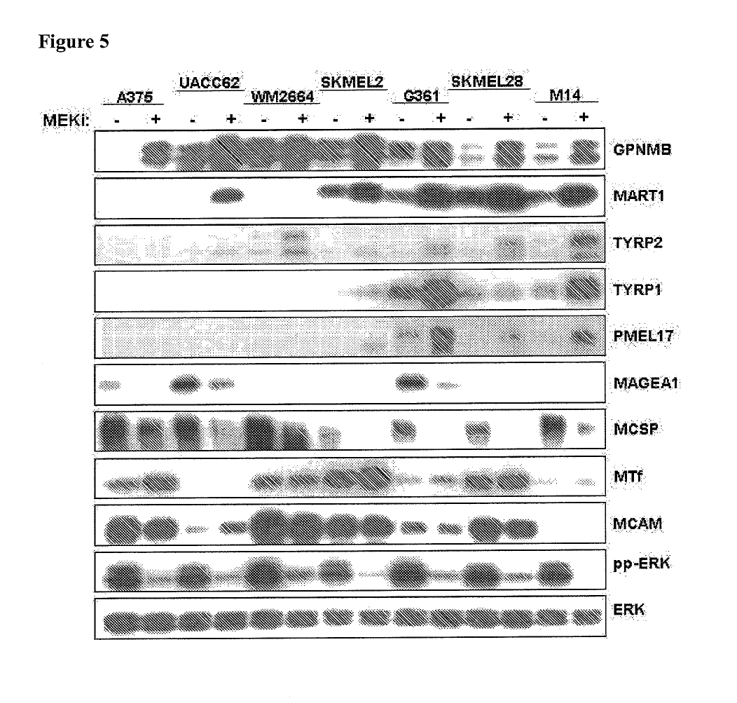

The induction of GPNMB in melanoma cell lines by a MEK inhibitor was compared to that of other melanoma-associated proteins by immunoblotting (FIG. 5). Results indicated that GPNMB was the most consistently induced melanoma target of those examined. Some of the other melanoma-associated targets (MART-1, TYRP-2, TYRP-1, PMEL17) were also induced by MEK inhibition in some cell lines, while other targets either were not effected or even decreased (MAGEA1, MCSP) in response to MEK inhibition.

### Example 8

### Identification of Compounds that Enhance GPNMB Expression in Melanoma and Glioblastoma Cell Lines Independently of NRAS/BRAF Mutational Status

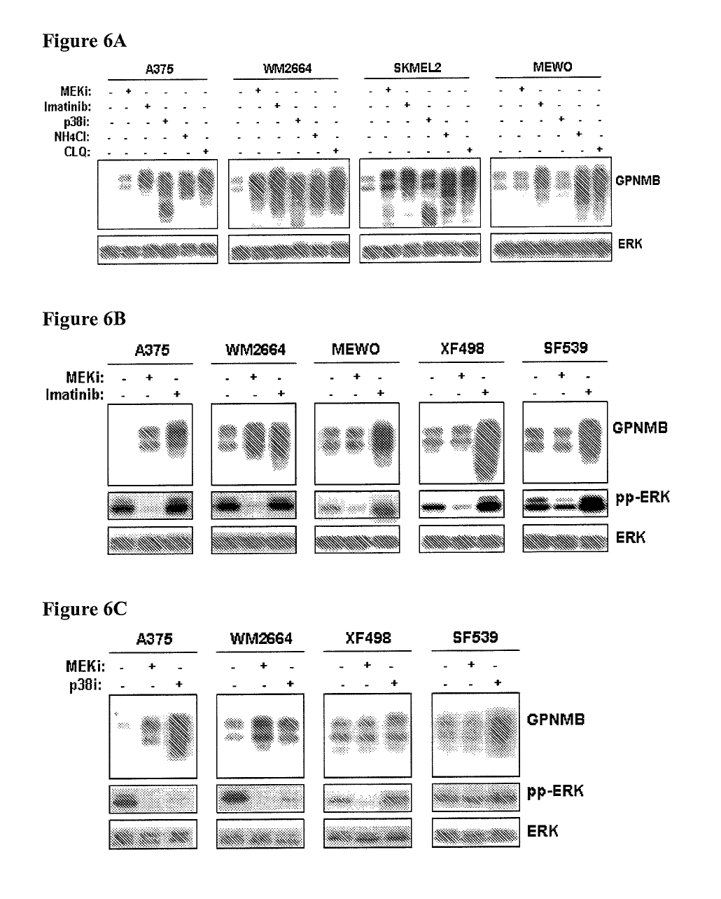

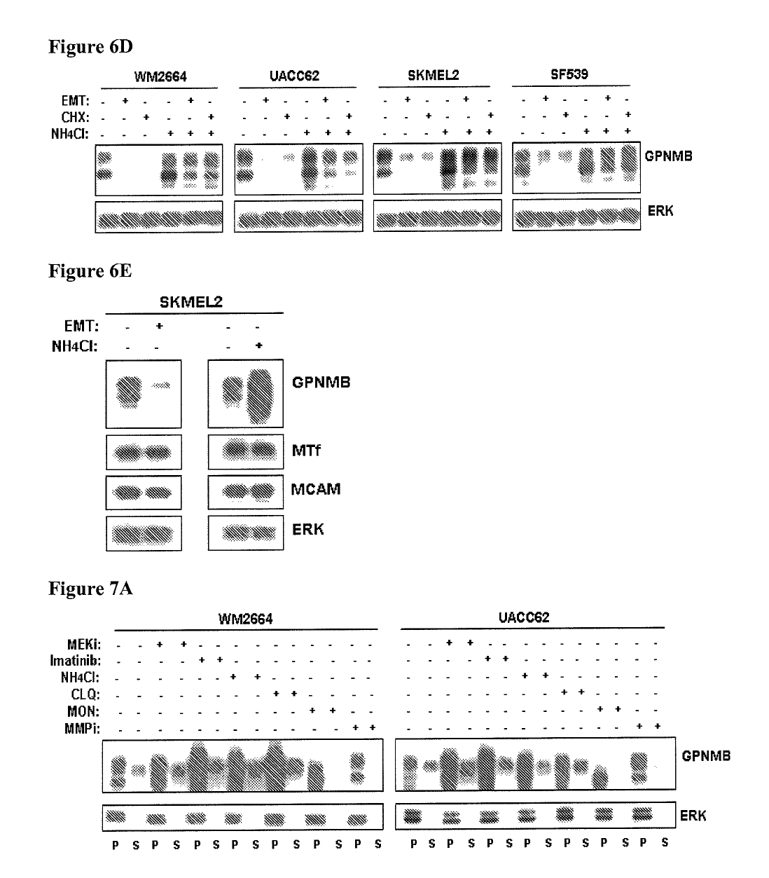

In the screen for compounds capable of inducing GPNMB expression, several compounds that increased the expression of this protein in both melanoma and glioblastoma cell lines, regardless of the presence of NRAS or BRAF mutations, were identified. Compounds falling into this category included imatinib, p38 MAPK inhibitors, ammonium chloride (NH4Cl) and chloroquine. GPNMB was induced by these compounds in melanoma cell lines harboring mutations in NRAS (SKMEL2) or BRAF (A375, WM2664), as well as in melanoma cell line (MEWO) possessing wild-type NRAS/BRAF (FIG. 6A). These compounds were also shown by flow cytometry to increase GPNMB surface-expression on SKMEL2 cells (Table 37, bottom left).

A close examination of the effects of imatinib shows that this compound induced robust GPNMB expression in a variety of melanoma and glioblastoma cell lines without inhibiting phospho-ERK (FIG. 6B). The inhibition of p38 MAPK similarly induced GPNMB expression in both melanoma and glioblastoma cell lines (albeit with some variability), but in contrast to imatinib, compounds that inhibited p38 MAPK decreased phospho-ERK levels in melanomas harboring mutations in BRAF (A375, WM2664) but not in the non-melanoma cell line examined (SF539 glioblastoma) that possesses wild-type NRAS/BRAF (FIG. 6C).

Ammonium chloride increases the pH of lysosomes/endosomes, thereby reducing the activity of the proteases residing in these organelles. Studies were designed to test whether this compound increased GPNMB expression by increasing the half-life of this protein. Cells were treated with a protein synthesis inhibitor (cyclohexamide or emetine) in the presence or absence of ammonium chloride, followed by immunoblotting for GPNMB (FIG. 6D). Cells treated for just 1 hour with a protein synthesis inhibitor in the absence of ammonium chloride expressed little or no GPNMB, indicating that this protein has a relatively short half-life. However, when ammonium chloride was included along with the protein synthesis inhibitor, GPNMB was once again readily detectable, thus supporting the hypothesis that ammonium chloride increased the expression of GPNMB via protein stabilization.

To examine whether a short half-life and stabilization by ammonium chloride are common attributes of melanoma-associated targets, a comparison of the effects of protein synthesis inhibition and ammonium chloride on the expression of GPNMB as well as two other melanoma-associated targets (MTf, MCAM) was performed (FIG. 6E). The results of this experiment showed that GPNMB possessed a shorter half-life and was more highly induced by ammonium chloride than were the two other melanoma-associated targets.

### Example 9

### Identification of Compounds that Inhibit GPNMB Shedding

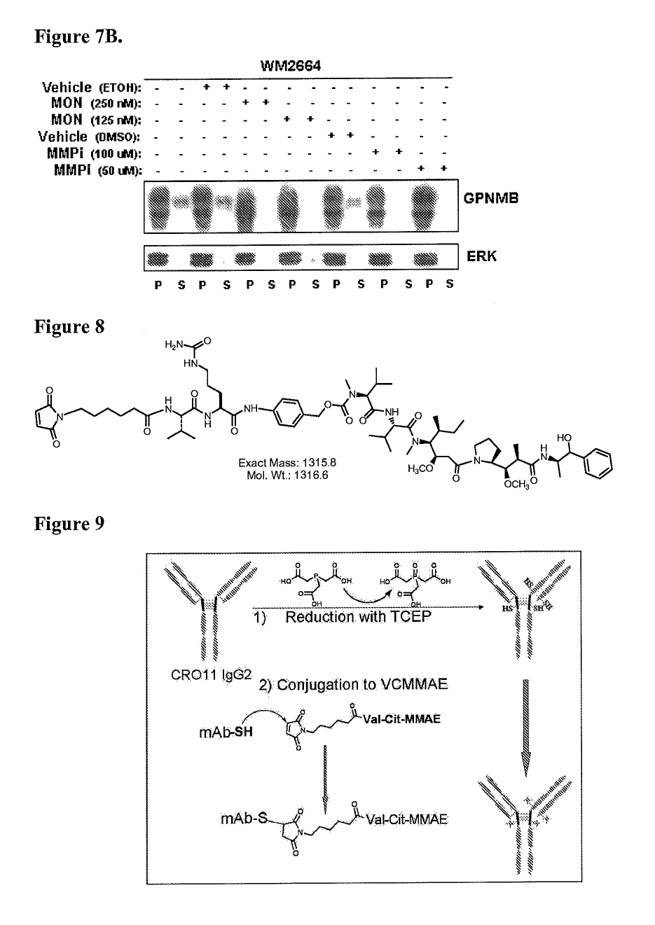

Shedding of membrane proteins is a common phenomenon that may affect ADC activity. To investigate potential GPNMB shedding, conditioned media collected from melanoma cell lines was immunoblotted for GPNMB (FIG. 7A). Results showed that a protein which migrated slightly faster than the slowest migrating ˜130 kDa cell-associated GPNMB species was readily detectable in the conditioned media of GPNMB-expressing melanoma cell lines (WM2664, UACC62). Shed GPNMB was also readily detected in conditioned media harvested from several other melanoma and glioblastoma cell lines examined, although a very low level of shed GPNMB was found in conditioned media harvested from A375 melanoma cells, a finding which is consistent with the low basal level of GPNMB expression by these cells. Next, the ability of various compounds to influence GPNMB shedding was examined, and two compounds that decreased or eliminated GPNMB shedding were identified (monensin and the metalloprotease inhibitor GM6001; FIG. 7A, 7B). Monensin also decreased or eliminated the slowest migrating cell-associated ˜130 kDa GPNMB species, and increased the expression of the faster migrating ˜110 kDa GPNMB species. Both monensin and GM6001 increased GPNMB surface expression on melanoma cells (Table 37, bottom right). These results indicate that GPNMB shedding occurs and can be decreased through pharmacological intervention.

### EQUIVALENTS

The foregoing description and Examples detail certain preferred embodiments of the antibodies and describes the best mode contemplated by the inventors. It will be appreciated, however, that no matter how detailed the foregoing may appear in text, the methods of making and using the antibodies described herein may be practiced in many ways and the invention should be construed in accordance with the appended claims and any equivalents thereof. The foregoing written specification is considered to be sufficient to enable one skilled in the art to practice the embodiments described herein.

What is claimed is:

1. A pharmaceutical composition comprising an isolated monoclonal antibody that specifically binds to GPNMB and a second agent that increases expression of GPNMB on a tumor cell or decreases shedding of GPNMB by a tumor cell, wherein the antibody comprises a heavy chain variable region comprising an amino acid sequence selected from the group consisting of: SEQ ID NO: 2, 20, 38, 56, 74, 92, 110, 128, 146, 164, 182, 200, 218, 236, 253, 256, 260, 265, 270, 274, 277, 281 and 285; and the antibody comprises a light chain variable region comprising an amino acid sequence selected from the group consisting of: SEQ ID NO: 11, 29, 47, 65, 83, 101, 119, 137, 155, 173, 191, 209, 227 and 245, and the second agent is selected from the group consisting of: an inhibitor of the ERK pathway, a tyrosine kinase inhibitor, an inhibitor of p38 MAPK, a lysosomotropic weak base and an inhibitor of GPNMB shedding.

2. The composition of claim 1, wherein the antibody is a human monoclonal antibody.

3. The composition of claim 1, wherein the antibody comprises
(a) a V_{H} CDR1 region comprising the amino acid sequence of SEQ ID NO: 4, 22, 40, 58, 76, 94, 112, 130, 148, 166, 184, 202, 220, 238, 254, 257, 261, 266, 271, 278, 282 or 286;
(b) a V_{H} CDR2 region comprising the amino acid sequence of SEQ ID NO: 6, 24, 42, 60, 78, 96, 114, 132, 150, 168, 186, 204, 222, 240, 255, 258, 262, 267, 272, 275, 279, 283 or 287;
(c) a V_{H} CDR3 region comprising the amino acid sequence of SEQ ID NO: 8, 26, 44, 62, 80, 98, 116, 134, 152, 170, 188, 206, 224, 242, 259, 263, 264, 268, 269, 273, 276, 280, 284 or 288;
(d) a V_{L} CDR1 region comprising the amino acid sequence of SEQ ID NO: 13, 31, 49, 67, 85, 103, 121, 139, 157, 175, 193, 211, 229 or 247;
(e) a V_{L} CDR2 region comprising the amino acid sequence of SEQ ID NO: 15, 33, 51, 69, 87, 105, 123, 141, 159, 177, 195, 213, 231, 249 or 279;
(f) a V_{L} CDR3 region comprising the amino acid sequence of SEQ ID NO: 17, 35, 53, 71, 89, 107, 125, 143, 161, 179, 197, 215, 233 or 251; and
wherein said antibody binds GPNMB.

4. The composition of claim 1, wherein said antibody is conjugated to a cytotoxic agent.

5. The composition of claim 4, wherein the cytotoxic agent is auristatin E (dolastatin-10) or a derivative thereof.

6. The composition of claim 1, wherein the tumor cell is a melanoma cell or a glioblastoma cell.

7. The composition of claim 1, wherein the ERK pathway inhibitor is selected from the group consisting of: A6355 (3-(2-Aminoethyl)-5-((4-ethoxyphenyl)methylene)-2,4-thiazolidinedione hydrochloride), and FR180204 ((5-(2-phenylpyrazolo[1,5-a]pyridin-3-yl)-1H-pyrazolo[3,4-c]pyridazin-3-amine)); the tyrosine kinase inhibitor is imatinib; the lysosomotropic weak base is ammonium chloride or chloroquine; the inhibitor of GPNMB shedding is monensin; and the p38 MAK inhibitor is SB202190 (4-[4-(4-Fluorophenyl)-5-(4-pyridinyl)-1H-imidazol-2-yl]phenol) or SB203580 (4-[5-(4-Fluorophenyl)-2-[4-(methylsulfonyl)phenyl]-1H-imidazol-4-yl]pyridine).

8. A method for enhancing expression of GPNMB on the surface of a tumor cell, comprising contacting a tumor cell with a composition comprising an isolated monoclonal antibody that specifically binds to GPNMB and a second agent selected from an inhibitor of the ERK pathway, a tyrosine kinase inhibitor, an inhibitor of p38 MAPK, a lysosomotropic weak base and an inhibitor of GPNMB shedding, wherein the composition is present in an amount sufficient to increase expression of GPNMB on the tumor cell or decrease shedding of GPNMB by the tumor cell.

9. The method of claim 8, wherein the antibody comprises a heavy chain variable region comprising an amino acid sequence selected from the group consisting of: SEQ ID NO: 2, 20, 38, 56, 74, 92, 110, 128, 146, 164, 182, 200, 218, 236, 253, 256, 260, 265, 270, 274, 277, 281 and 285; and the antibody comprises a light chain variable region comprising an amino acid sequence selected from the group consisting of: SEQ ID NO: 11, 29, 47, 65, 83, 101, 119, 137, 155, 173, 191, 209, 227 and 245.

10. The method of claim 9, wherein the antibody comprises
(a) a V_{H} CDR1 region comprising the amino acid sequence of SEQ ID NO: 4, 22, 40, 58, 76, 94, 112, 130, 148, 166, 184, 202, 220, 238, 254, 257, 261, 266, 271, 278, 282, or 286;
(b) a V_{H} CDR2 region comprising the amino acid sequence of SEQ ID NO: 6, 24, 42, 60, 78, 96, 114, 132, 150, 168, 186, 204, 222, 240, 255, 258, 262, 267, 272, 275, 279, 283, or 287;
(c) a V_{H} CDR3 region comprising the amino acid sequence of SEQ ID NO: 8, 26, 44, 62, 80, 98, 116, 134, 152, 170, 188, 206, 224, 242, 259, 263, 264, 268, 269, 273, 276, 280, 284, or 288;
(d) a V_{L} CDR1 region comprising the amino acid sequence of SEQ ID NO: 13, 31, 49, 67, 85, 103, 121, 139, 157, 175, 193, 211, 229, or 247;
(e) a V_{L} CDR2 region comprising the amino acid sequence of SEQ ID NO: 15, 33, 51, 69, 87, 105, 123, 141, 159, 177, 195, 213, 231, 249, or 279;
(f) a V_{L} CDR3 region comprising the amino acid sequence of SEQ ID NO: 17, 35, 53, 71, 89, 107, 125, 143, 161, 179, 197, 215, 233, or 251; and
wherein said antibody binds GPNMB.

11. The method of claim 8, wherein said antibody is conjugated to auristatin E (dolastatin-10) or a derivative thereof.

12. The method of claim 8, wherein the tumor cell is a glioblastoma cell or a melanoma cell comprising the NRAS or BRAF mutation.

13. The method of claim 8, wherein the second agent is selected from the group consisting of: A6355 (3-(2-Aminoethyl)-5-(4-ethoxyphenyl)methylene)-2,4-thiazolidinedione hydrochloride); FR180204 ((5-(2-phenylpyrazolo[1,5-a]pyridin-3-yl)-1H-pyrazolo[3,4-c]pyridazin-3-amine)); imatinib; ammonium chloride; chloroquine; monensin; SB202190 (4-[4-(4-Fluorophenyl)-5-(4-pyridinyl)-1H-imidazol-2-yl]phenol); and SB203580 (4-[5-(4-Fluorophenyl)-2-[4-(methylsulfonyl)phenyl]-1H-imidazol-4-yl]pyridine).

14. A method of treating cancer associated with overexpression of GPNMB, comprising administering to a subject in need thereof an effective amount of a composition comprising an isolated monoclonal antibody that specifically binds to GPNMB and a second agent selected from the group consisting of: an inhibitor of the ERK pathway, a tyrosine kinase inhibitor, an inhibitor of p38 MAPK, a lysosomotropic weak base and an inhibitor of GPNMB shedding.

15. The method of claim 14, wherein said cancer is melanoma or a neoplasm of CNS system.

16. The method of claim 14, wherein the antibody comprises a heavy chain variable region comprising an amino acid sequence selected from the group consisting of: SEQ ID NO: 2, 20, 38, 56, 74, 92, 110, 128, 146, 164, 182, 200, 218, 236, 253, 256, 260, 265, 270, 274, 277, 281, and 285; and the antibody comprises a light chain variable region comprising an amino acid sequence selected from the group consisting of: SEQ ID NO: 11, 29, 47, 65, 83, 101, 119, 137, 155, 173, 191, 209, 227, and 245.

17. The method of claim 14, wherein the antibody comprises
(a) a V_{H} CDR1 region comprising the amino acid sequence of SEQ ID NO: 4, 22, 40, 58, 76, 94, 112, 130, 148, 166, 184, 202, 220, 238, 254, 257, 261, 266, 271, 278, 282, or 286;
(b) a V_{H} CDR2 region comprising the amino acid sequence of SEQ ID NO: 6, 24, 42, 60, 78, 96, 114, 132, 150, 168, 186, 204, 222, 240, 255, 258, 262, 267, 272, 275, 279, 283, or 287;
(c) a V_{H} CDR3 region comprising the amino acid sequence of SEQ ID NO: 8, 26, 44, 62, 80, 98, 116, 134, 152, 170, 188, 206, 224, 242, 259, 263, 264, 268, 269, 273, 276, 280, 284, or 288;
(d) a V_{L} CDR1 region comprising the amino acid sequence of SEQ ID NO: 13, 31, 49, 67, 85, 103, 121, 139, 157, 175, 193, 211, 229, or 247;
(e) a V_{L} CDR2 region comprising the amino acid sequence of SEQ ID NO: 15, 33, 51, 69, 87, 105, 123, 141, 159, 177, 195, 213, 231, 249, or 279;
(f) a V_{L} CDR3 region comprising the amino acid sequence of SEQ ID NO: 17, 35, 53, 71, 89, 107, 125, 143, 161, 179, 197, 215, 233, or 251; and
wherein said antibody binds GPNMB.

18. The method of claim 14, wherein said antibody is conjugated to auristatin E (dolastatin-10) or a derivative thereof.

19. The method of claim 14, wherein the tumor cell is a glioblastoma cell or a melanoma cell comprising the NRAS or BRAF mutation.

20. The method of claim 14, wherein the second agent is selected from the group consisting of: A6355 (3-(2-Aminoethyl)-5-((4-ethoxyphenyl)methylene)-2,4-thiazolidinedione hydrochloride); FR180204 ((5-(2-phenylpyrazolo[1,5-a]pyridin-3-yl)-1H-pyrazolo[3,4-c]pyridazin-3-amine)); imatinib; ammonium chloride; chloroquine; monensin; SB202190 (4-[4-(4-Fluorophenyl)-5-(4-pyridinyl)-1H-imidazol-2-yl]phenol); and SB203580 (4-[5-(4-Fluorophenyl)-2-[4-(methylsulfonyl)phenyl]-1H-imidazol-4-yl]pyridine).
